(19) United States
(12) Patent Application Publication
(10) Pub. No.: US 2026/0070906 A1
(43) Pub. Date: Mar. 12, 2026

(54) IMIDAZO[4,5-C]PYRIDINE COMPOUNDS FOR THE TREATMENT OF CANCER

(71) Applicant: Hoffmann-La Roche Inc., Little Falls, NJ (US)

(72) Inventors: Jianping WANG, Shanghai (CN); Junjie WANG, Shanghai (CN); Kuan WANG, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 19/395,255

(22) Filed: Nov. 20, 2025

Related U.S. Application Data
(63) Continuation of application No. PCT/EP2024/063876, filed on May 21, 2024.

(30) Foreign Application Priority Data
May 22, 2023 (WO) PCT/CN2023/095563

(51) Int. Cl. C07D 471/04; A61K 31/437; A61K 31/444; A61K 31/4725; A61K 31/496

(52) U.S. Cl. CPC C07D 471/04; A61K 31/437; A61K 31/444; A61K 31/4725; A61K 31/496

(57) ABSTRACT

The present invention relates to compounds of formula (I),

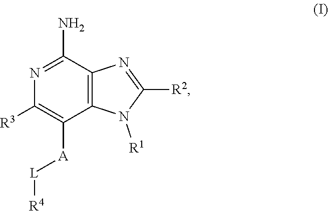

wherein R¹ to R⁴, A and L are as described herein, and their pharmaceutically acceptable salt thereof, and compositions including the compounds and methods of using the compounds.

The present invention relates to organic compounds useful for therapy and/or prophylaxis in a mammal, and in particular to agonism of TLR7 and TLR8 useful for treating cancers.

### FIELD OF THE INVENTION

The present invention relates to novel imidazo[4,5-c]pyridine compounds and their derivatives that have Toll-like receptor agonism activity thereof, as well as their manufacture, pharmaceutical compositions containing them and their potential use as medicaments for the treatment and/or prophylaxis of cancers.

Toll-like receptors (TLRs) are a family of transmembrane proteins that recognize structurally conserved molecules that are derived from and unique to pathogens, referred to as pathogen-associated molecular patterns (PAMPS). They play an important role in sensing invading pathogens and subsequent initiation of innate immune responses. There are 10 known members of the TLR family in humans, which are type I transmembrane proteins featuring an extracellular leucine-rich domain and a cytoplasmic tail that contains a conserved Toll/interleukin (IL)-1 receptor (TIR) domain. Within this family, TLR3, TLR7, TLR8 and TLR9 are located within endosomes. In the activated dimeric receptor configuration TLR7 and TLR8 recognize single strand RNA at one ligand binding site and the ribonucleoside degradation products guanosine and uridine, respectively, (as well as small molecule ligands with related structural motifs) at a second ligand binding site (Zhang et al 2016 Immunity 45(4); 737-748: Tanji et al 2015 Nat Struct Mol Biol 22: 109-115).

As a part of the innate immune system, myeloid cells are capable of reacting to infection and aberrant cell behavior. They are the first to detect the pathogen/aberrant cells and eliminate them by different mechanisms including inflammatory cytokine secretion and phagocytosis.

Sustained activation of T cells by providing co-stimulatory signals and antigen presentation to T cells is another pivotal function of myeloid cells paving way to a long lasting immune response. On the other hand, they also play a key role in tissue repair and homeostasis by suppressing uncontrolled immune activation. Tumors take advantage of this property of these cells by recruiting them where they create a suppressive microenvironment thereby inhibiting T cells by direct and indirect mechanisms. Tumor associated myeloid cells can still be activated in the suppressive tumor microenvironment by delivering the right signals for e.g. TLR agonists. Upon activation, they can produce inflammatory cytokines and upregulate activation markers and thus trigger an anti-tumor immune response, ideally resulting in long lasting adaptive response.

TLR7 is predominantly expressed in plasmacytoid dendritic cells and B cells and agonism of TLR7 leads to the induction of interferon α/β, which plays essential functions in the control of the adaptive immune response (Bao and Liu 2013 Protein Cell 4:40-5). TLR8 is expressed in myeloid dendritic cells, monocytes, macrophages, and neutrophils. Activation of TLR8 induces a prominent pro-inflammatory cytokine profile, characterized by increased production of tumor necrosis factor-α, interleukin-12, and IL-18 (Eigenbrod et al J Immunol, 2015, 195, 1092-1099). Most antigen presenting cell types express only one of these two receptors, accordingly small molecules with potent agonist activity against both TLR7 and TLR8 receptors are potentially more effective immune adjuvants than TLR7 agonists alone. Thus, a TLR7/TLR8 (TLR7/8) small molecule agonist with dual bioactivity could provide further benefit over a more selective TLR7 agonist and would cause innate immune responses in a wider range of antigen presenting cells and other key immune cell types, which could stimulate effective anti-tumor responses in cancer.

In fact, several identified TLR7/8 agonists have been considered for therapeutic purposes. The TLR7/8 dual agonist Resiquimod (R-848) is an immune modulating topical gel in phase II clinical trials at Galderma for the treatment of stage cutaneous T-cell lymphoma. BDB-001 is a TLR 7/8 dual agonist in phase II clinical trials at Seven and Eight Biopharmaceuticals as an intravenous treatment for advanced or metastatic solid tumors refractory to anti-PD-1/anti-PD-L1 therapy. Despite the success of Imiquimod (Aldera) in treating superficial basal cell carcinoma, there remains high unmet clinical need for not only more potent TLR7 agonists, but also balanced, potent TLR7/8 agonists to expand treatment options for patients for various cancers.

### SUMMARY OF THE INVENTION

The present invention relates to novel compounds of formula (I),

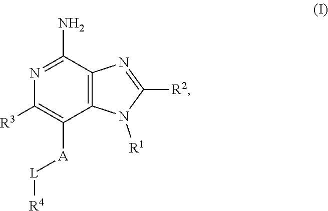

wherein
R¹ is H, C₁₋₆alkyl or hydroxyC₁₋₆alkyl;
R² is C₁₋₆alkyl or C₁₋₆alkoxyC₁₋₆alkyl;
R³ is C₁₋₆alkyl;
R⁴ is 1H-indazolyl substituted by (C₁₋₆alkyl)₂aminoC₁₋₆alkyl,
   1H-indolyl substituted by (C₁₋₆alkyl)₂aminoC₁₋₆alkyl,
   1H-isoquinolinyl twice substituted by C₁₋₆alkyl and hydroxy, phenyl which is once, twice or three times substituted by substituents independently selected from (C₁-6alkyl)₂aminoC₁₋₆alkyl, (C₁₋₆alkylpiperazinyl)C₁-6alkyl, amino, C₁₋₆alkoxy, C₁₋₆alkyl, hydroxy and pyrrolidinylC₁₋₆alkyl, or pyridyl substituted by (C₁₋₆alkyl)₂aminoC₁₋₆alkylamino;
A is S or Se;
L is a bond or C₁₋₆alkylene;
or a pharmaceutically acceptable salt thereof.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “C₁₋₆alkoxy” denotes C₁₋₆alkyl-O—.

The term “halogen” and “halo” are used interchangeably herein and denote fluoro, chloro, bromo, or iodo.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### AGONIST OF TLR7 and TLR8

The present invention relates to (i) a compound of formula (I),

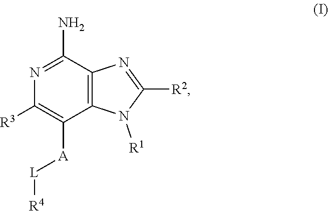

wherein
R¹ is H, C₁₋₆alkyl or hydroxyC₁₋₆alkyl;
R² is C₁₋₆alkyl or C₁₋₆alkoxyC₁₋₆alkyl;
R³ is C₁₋₆alkyl;
R⁴ is 1H-indazolyl substituted by (C₆alkyl)₂aminoC₆alkyl,
   1H-indolyl substituted by (C₁₋₆alkyl)₂aminoC₁₋₆alkyl,
   1H-isoquinolinyl twice substituted by C₁₋₆alkyl and hydroxy,
   phenyl which is once, twice or three times substituted by substituents independently selected from (C₁₋₆alkyl)₂aminoC₁₋₆alkyl, (C₁₋₆alkylpiperazinyl)C₁₋₆alkyl, amino, C₁₋₆alkoxy, C₁₋₆alkyl, hydroxy and pyrrolidinylC₁₋₆alkyl, or pyridyl substituted by (C₁₋₆alkyl)₂aminoC₁₋₆alkylamino;
A is S or Se;
L is a bond or C₁₋₆alkylene;
or a pharmaceutically acceptable salt thereof.

A further embodiment of present invention is (ii) a compound of formula (I) according to (i), or a pharmaceutically acceptable salt thereof, wherein R¹ is H or C₁₋₆alkyl.

A further embodiment of present invention is (iii) a compound of formula (I), according to (i) or (ii), or a pharmaceutically acceptable salt thereof, wherein R¹ is H or methyl.

A further embodiment of present invention is (iv) a compound of formula (I) according to any one of (i) to (iii), wherein R² is butyl or ethoxymethyl.

A further embodiment of present invention is (v) a compound of formula (I), or a pharmaceutically acceptable salt thereof, according to any one of (i) to (iv), wherein R³ is methyl.

A further embodiment of present invention is (vi) a compound of formula (I), or a pharmaceutically acceptable salt thereof, according to any one of (i) to (v), wherein A is S.

A further embodiment of present invention is (vii) a compound of formula (I), or a pharmaceutically acceptable salt thereof, according to any one of (i) to (vi), wherein L is a bond or ethylene.

A further embodiment of present invention is (viii) a compound of formula (I), or a pharmaceutically acceptable salt thereof, according to any one of (i) to (vii), wherein R⁴ is phenyl which is once, twice or three times substituted by substituents independently selected from (C₁₋₆alkyl)₂aminoC₁₋₆alkyl, amino, C₁₋₆alkoxy, hydroxy and pyrrolidinylC₁₋₆alkyl.

A further embodiment of present invention is (ix) a compound of formula (I), or a pharmaceutically acceptable salt thereof, according to any one of (i) to (viii), wherein R⁴ is phenyl which is once, twice or three times substituted by substituents independently selected from (dimethylamino)methyl, [ethyl(methyl)amino]methyl, [ethyl(propyl)amino]methyl, amino, hydroxy, methoxy and pyrrolidinylmethyl.

A further embodiment of present invention is (x) a compound of formula (I), or a pharmaceutically acceptable salt thereof, according to any one of (i) to (ix), wherein R⁴ is 2-[4-[(dimethylamino)methyl]-3-hydroxy-phenyl]ethyl, 2-[4-[(dimethylamino)methyl]phenyl]ethyl, 2-amino-4-(pyrrolidin-1-ylmethyl)phenyl, 4-[(dimethylamino)methyl]-2,6-dimethoxy-phenyl, 4-[(dimethylamino)methyl]-2-hydroxy-phenyl, 4-[(dimethylamino)methyl]-3-hydroxy-phenyl, 4-[(dimethylamino)methyl]phenyl, 4-[[ethyl(methyl)amino]methyl]phenyl or 4-[[ethyl(propyl)amino]methyl]phenyl.

A further embodiment of present invention is (xi) a compound of formula (I), according to any one of (i) to (x), wherein
R¹ is H or C₁₋₆alkyl;
R² is C₁₋₆alkyl or C₁₋₆alkoxyC₁₋₆alkyl;
R³ is C₁₋₆alkyl;
R⁴ is phenyl which is once, twice or three times substituted by substituents independently selected from (C₁₋₆alkyl)₂aminoC₁₋₆alkyl, amino, C₁₋₆alkoxy, hydroxy and pyrrolidinylC₁₋₆alkyl;
A is S;
L is a bond or C₁₋₆alkylene;
or a pharmaceutically acceptable salt thereof.

A further embodiment of present invention is (xii) a compound of formula (I), according to any one of (i) to (xi), wherein
R¹ is H or methyl;
R² is butyl or ethoxymethyl;
R³ is methyl;
R⁴ is 2-[4-[(dimethylamino)methyl]-3-hydroxy-phenyl]ethyl, 2-[4-[(dimethylamino)methyl]phenyl]ethyl, 2-amino-4-(pyrrolidin-1-ylmethyl)phenyl, 4-[(dimethylamino)methyl]-2,6-dimethoxy-phenyl, 4-[(dimethylamino)methyl]-2-hydroxy-phenyl, 4-[(dimethylamino)methyl]-3-hydroxy-phenyl, 4-[(dimethylamino)methyl]phenyl, 4-[[ethyl(methyl)amino]methyl]phenyl or 4-[[ethyl(propyl)amino]methyl]phenyl;
A is S;
L is a bond or ethylene;
or a pharmaceutically acceptable salt thereof.

Another embodiment of present invention is a compound of formula (I) selected from the following:
7-[4-[(dimethylamino)methyl]phenyl]sulfanyl-2-(ethoxymethyl)-6-methyl-1H-imidazo[4,5-c]pyridin-4-amine;
1-[4-amino-2-butyl-7-[4-[(dimethylamino)methyl]phenyl]sulfanyl-6-methyl-imidazo[4,5-c]pyridin-1-yl]-2-methyl-propan-2-ol;
1-[4-amino-7-[4-[(dimethylamino)methyl]phenyl]selanyl-2-(ethoxymethyl)-6-methyl-imidazo[4,5-c]pyridin-1-yl]-2-methyl-propan-2-ol;
7-[4-[(dimethylamino)methyl]-2,6-dimethoxy-phenyl]sulfanyl-2-(ethoxymethyl)-6-methyl-1H-imidazo[4,5-c]pyridin-4-amine;
2-(ethoxymethyl)-7-[4-[[ethyl(propyl)amino]methyl]phenyl]sulfanyl-6-methyl-1H-imidazo[4,5-c]pyridin-4-amine;
2-(ethoxymethyl)-7-[4-[[ethyl(methyl)amino]methyl]phenyl]sulfanyl-6-methyl-1H-imidazo[4,5-c]pyridin-4-amine;
7-[4-[(dimethylamino)methyl]phenyl]sulfanyl-2-(ethoxymethyl)-1,6-dimethyl-imidazo[4,5-c]pyridin-4-amine;
2-[[4-amino-2-(ethoxymethyl)-6-methyl-1H-imidazo[4,5-c]pyridin-7-yl]sulfanyl]-5-[(dimethylamino)methyl]phenol;
7-[4-[(dimethylamino)methyl]-3-methoxy-phenyl]sulfanyl-2-(ethoxymethyl)-6-methyl-1H-imidazo[4,5-c]pyridin-4-amine;
7-[2-[4-[(dimethylamino)methyl]phenyl]ethylsulfanyl]-2-(ethoxymethyl)-6-methyl-1H-imidazo[4,5-c]pyridin-4-amine;
5-[[4-amino-2-(ethoxymethyl)-6-methyl-1H-imidazo[4,5-c]pyridin-7-yl]sulfanyl]-2-[(dimethylamino)methyl]phenol;
5-[4-amino-2-(ethoxymethyl)-1,6-dimethyl-imidazo[4,5-c]pyridin-7-yl]sulfanyl-2-[(dimethylamino)methyl]phenol;
5-[2-[[4-amino-2-(ethoxymethyl)-6-methyl-1H-imidazo[4,5-c]pyridin-7-yl]sulfanyl]ethyl]-2-[(dimethylamino)methyl]phenol;
2-butyl-7-[4-[(dimethylamino)methyl]phenyl]sulfanyl-6-methyl-1H-imidazo[4,5-c]pyridin-4-amine;
2-butyl-7-[2-[4-[(dimethylamino)methyl]phenyl]ethylsulfanyl]-6-methyl-1H-imidazo[4,5-c]pyridin-4-amine;
6-[[4-amino-2-(ethoxymethyl)-6-methyl-1H-imidazo[4,5-c]pyridin-7-yl]sulfanyl]-3-[(dimethylamino)methyl]-2-methyl-phenol;
2-[[4-amino-2-(ethoxymethyl)-6-methyl-1H-imidazo[4,5-c]pyridin-7-yl]sulfanyl]-5-(diethylaminomethyl)phenol;
7-[[4-[(dimethylamino)methyl]-1H-indol-7-yl]sulfanyl]-2-(ethoxymethyl)-6-methyl-1H-imidazo[4,5-c]pyridin-4-amine;
7-[[4-[(dimethylamino)methyl]-1H-indazol-7-yl]sulfanyl]-2-(ethoxymethyl)-6-methyl-1H-imidazo[4,5-c]pyridin-4-amine;
7-[2-amino-4-[(dimethylamino)methyl]phenyl]sulfanyl-2-(ethoxymethyl)-6-methyl-1H-imidazo[4,5-c]pyridin-4-amine;
2-[(4-amino-2-butyl-6-methyl-1H-imidazo[4,5-c]pyridin-7-yl)sulfanyl]-5-[(dimethylamino)methyl]phenol;
2-[[4-amino-2-(ethoxymethyl)-6-methyl-1H-imidazo[4,5-c]pyridin-7-yl]sulfanyl]-5-(pyrrolidin-1-ylmethyl)phenol;
2-[[4-amino-2-(ethoxymethyl)-6-methyl-1H-imidazo[4,5-c]pyridin-7-yl]sulfanyl]-5-[(dimethylamino)methyl]-4-methyl-phenol;
2-[[4-amino-2-(ethoxymethyl)-6-methyl-1H-imidazo[4,5-c]pyridin-7-yl]sulfanyl]-5-[1-(dimethylamino)ethyl]phenol;
5-[[4-amino-2-(ethoxymethyl)-6-methyl-1H-imidazo[4,5-c]pyridin-7-yl]sulfanyl]-2-[(dimethylamino)methyl]-4-methyl-phenol;
7-[2-amino-4-(pyrrolidin-1-ylmethyl)phenyl]sulfanyl-2-(ethoxymethyl)-6-methyl-1H-imidazo[4,5-c]pyridin-4-amine;
7-[2-amino-4-[(4-methylpiperazin-1-yl)methyl]phenyl]sulfanyl-2-(ethoxymethyl)-6-methyl-1H-imidazo[4,5-c]pyridin-4-amine;
7-[3-amino-4-[(4-methylpiperazin-1-yl)methyl]phenyl]sulfanyl-2-(ethoxymethyl)-6-methyl-1H-imidazo[4,5-c]pyridin-4-amine;
7-[2-amino-6-methoxy-4-[(4-methylpiperazin-1-yl)methyl]phenyl]sulfanyl-2-(ethoxymethyl)-6-methyl-1H-imidazo[4,5-c]pyridin-4-amine;
N-[5-[[4-amino-2-(ethoxymethyl)-6-methyl-1H-imidazo[4,5-c]pyridin-7-yl]sulfanyl]-2-pyridyl]-N,N-dimethyl-ethane-1,2-diamine;
7-[4-[2-(dimethylamino)ethyl]phenyl]sulfanyl-2-(ethoxymethyl)-6-methyl-1H-imidazo[4,5-c]pyridin-4-amine; and
6-[[4-amino-2-(ethoxymethyl)-6-methyl-1H-imidazo[4,5-c]pyridin-7-yl]sulfanyl]-2-methyl-3,4-dihydro-1H-isoquinolin-7-ol;
   or a pharmaceutically acceptable salt thereof.

Another embodiment of present invention is related to (xvi) a process for the preparation of a compound according to any one of (i) to (xiii) comprising any one of the following steps:
a) the formation of compound of formula (I-1),

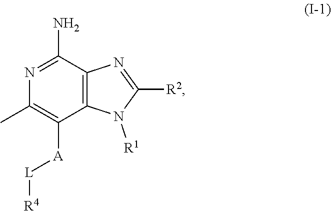

via reaction between compound of formula (III),

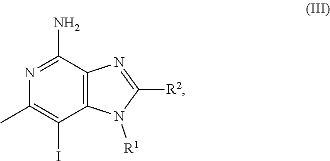

and compound of formula (II-1), WS-L-R⁴ (II-1), in the presence of a set of catalyst, ligand, reagent and base;
b) the formation of compound of formula (I-1) via deprotection of compound of formula (XIII),

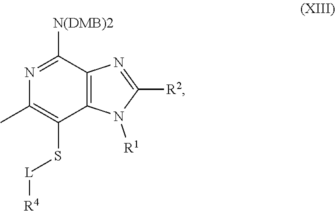

in the presence of an acid;
c) the formation of compound of formula (1-2),

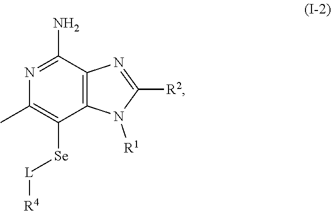

via the reaction between compound of formula (III) and compound of formula (II-2), B(OH)₂-L-R⁴ (II-2), in the presence of reagents;
wherein the set of catalyst, ligand, reagent and base in step a) is (1) Pd₂(dba)₃, XantPhos and DIEA (or Cs₂CO₃), (2) CuI, Mtpy and KOH, (3) Cu₂S, Fe and K₂CO₃ (or Cs₂CO₃), or (4) TIPSSH, XantPhos Pd G3 and K₂CO₃ (or Cs₂CO₃);
the acid in step b) is TFA or HCl;
the reagents in step c) are SeO₂ and KI;
wherein W is H, —S-L-R⁴ or R^{a}; R^{a} is alkoxycarbonylalkyl; R¹, R², and R⁴ are defined as in any one of (i) to (xii).

Another embodiment of present invention is (xv) a compound or pharmaceutically acceptable salt according to any one of (i) to (xiii), when manufactured according to the process of (xiv).

Another embodiment of present invention is (xvi) a pharmaceutical composition comprising a compound in accordance with any one of (i) to (xiii) and a pharmaceutically acceptable excipient.

Another embodiment of present invention is (xvii) a compound or pharmaceutically acceptable salt according to any one of (i) to (xiii) for use as therapeutically active substance.

Another embodiment of present invention is (xviii) the use of a compound according to any one of (i) to (xiii) as an agonist of TLR7 and TLR8.

Another embodiment of present invention is (xix) the use of a compound according to any one of (i) to (xiii) for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

Another embodiment of present invention is (xx) the use of a compound according to any one of (i) to (xiii) for the preparation of a medicament for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

Another embodiment of present invention is (xxi) the use of a compound according to any one of (xix) or (xx), wherein the cancer is selected from pancreatic ductal adenocarcinoma and colorectal carcinoma.

Another embodiment of present invention is (xxii) a method for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma, which method comprises administering a therapeutically effective amount of a compound as defined in any one of (i) to (xiii).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to activate TLR7/8 receptors and lead to produce TNF-α and other cytokines, which can be used, but not limited, for the treatment of cancers. For example, such amount may be below the amount that is toxic to normal cells, or the mammal as a whole.

In one example, the pharmaceutically effective amount of the compound of the invention administered parenterally per dose will be in the range of about 0.01 to 1000 mg/kg, alternatively about 0.01 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 1 to 500 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of a hyperproliferative disease. Another embodiment includes a pharmaceutical composition comprising a compound of formula (I) for use in the treatment of cancer.

The following embodiments illustrate typical compositions of the present invention, but serve merely as representative thereof.

### Composition A

A compound of the present invention can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Composition B

A compound of the present invention can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

### Indications and Methods of Treatment

The compounds of the invention target the TLR7 and TLR8. Accordingly, the compounds of the invention are useful for promoting the activation of antigen presenting cells (APC), contributing to the priming, trafficking and infiltration of T cells, promoting Th1 and CD8+T response, and enhancing the killing of cancer. Compounds of the invention are useful for boosting innate immune response in myeloid cells that express TLR7 and TLR8. Alternatively, compounds of the invention are useful for invigorate T cells in tumors in which the T cell present but suppressed, for example by upregulation of co-stimulatory molecules and production of pro-inflammatory cytokines. More broadly, the compounds can be used for the treatment of cancer types which are non-inflamed, e.g. immune desert or immune excluded by enhancing antigen presentation, T cell priming, recruitment/infiltration, and tumor killing.

Another embodiment includes a method of treating or preventing cancer in a mammal in need of such treatment, wherein the method comprises administering to said mammal a therapeutically effective amount of a compound of formula (I), a stereoisomer, tautomer, or pharmaceutically acceptable salt thereof. Cancer herein includes but is not limited to advanced solid tumors such as pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma, squamous cell carcinoma.

### Synthesis

The compounds of the present invention can be prepared by any conventional means. Suitable processes for synthesizing these compounds as well as their starting materials are provided in the schemes below and in the examples. All substituents, in particular, R¹ to R⁴ are as defined above unless otherwise indicated. Furthermore, and unless explicitly otherwise stated, all reactions, reaction conditions, abbreviations and symbols have the meanings well known to a person of ordinary skill in organic chemistry.

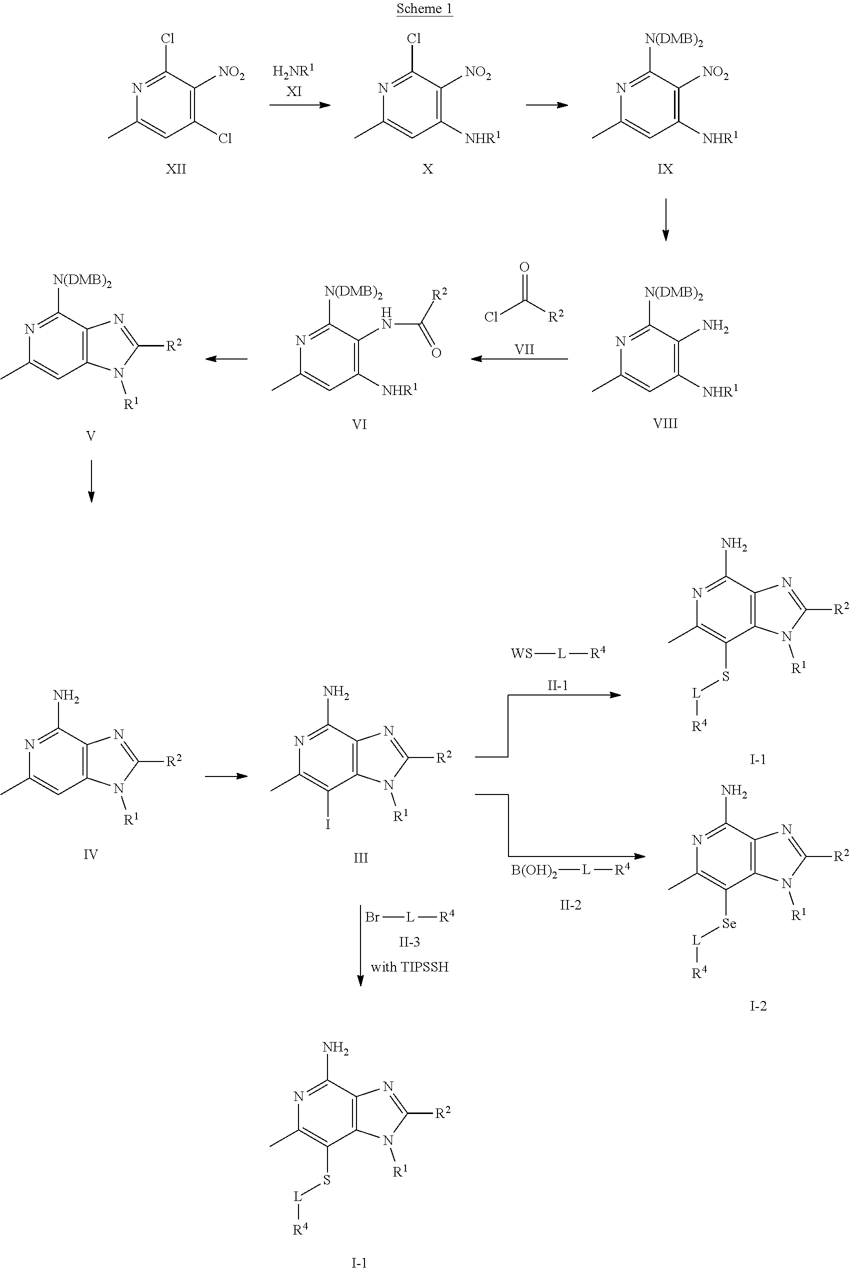

wherein W is H, —S-L-R⁴ or R^{a}; R^{a} is alkoxycarbonylalkyl.

The compounds of formula (I-1) and (I-2) can be prepared according to Scheme 1.

A compound of formula (X) is prepared by substitution of a compound of formula (XII) and a compound of formula (XI) in the presence of a suitable base, such as NH₃ or DIEA. A compound of formula (X) is converted to a compound of formula (IX) by introduction of 2,4-dimethoxyphenyl)methyl protecting group. A compound of formula (IX) can be reduced by a reducing agent, such as Pd/C with H₂, Pd(OH)₂ with H₂, and Fe with NH₄Cl to give a compound of formula (VIII). Condensation of a compound of formula (VIII) with a compound of formula (VII) in the presence of a suitable base, such as DIEA and NEt₃, affords a compound of formula (VI). A compound of formula (V) is prepared by reaction of a compound of formula (VI) in the presence of a suitable base, such as NaOH and KOH. Deprotection of a compound of formula (V) affords a compound of formula (IV) in the presence of a suitable acid such as TFA and HCl. Iodization of a compound of formula (IV) affords a compound of formula (III) in the presence of a suitable reagent such as NIS.

A compound of formula (I-1) can be prepared by coupling of a compound of formula (III) and a compound of formula (II-1) in the presence of a suitable catalyst such as Pd₂(dba)₃ with a suitable ligand such as XantPhos, and a suitable base such as DIEA or Cs₂CO₃. A compound of formula (I-1) can also be prepared by coupling of a compound of formula (III) and a compound of formula (II-1) in the presence of a suitable catalyst such as CuI with a suitable ligand such as Mtpy, and a suitable base such as KOH. A compound of formula (I-1) can also be prepared by coupling of a compound of formula (III) and a compound of formula (II-1) in the presence of a suitable reagent such as Cu₂S, Fe and a suitable base, such as K₂CO₃ and Cs₂CO₃. Alternatively a compound of formula (I-1) can be prepared by coupling of a compound of formula (III) and a compound of formula (II-3) in the presence of a suitable reagent, such as TIPSSH, and a suitable catalyst, such as XantPhos Pd G3, and a suitable base such as K₂CO₃ and Cs₂CO₃, in a suitable solvent such as 1,4-dioxane.

A compound of formula (I-2) can be prepared by reaction of a compound of formula (III) and a compound of formula (II-2) in the presence of suitable reagents, such as SeO₂ and KI.

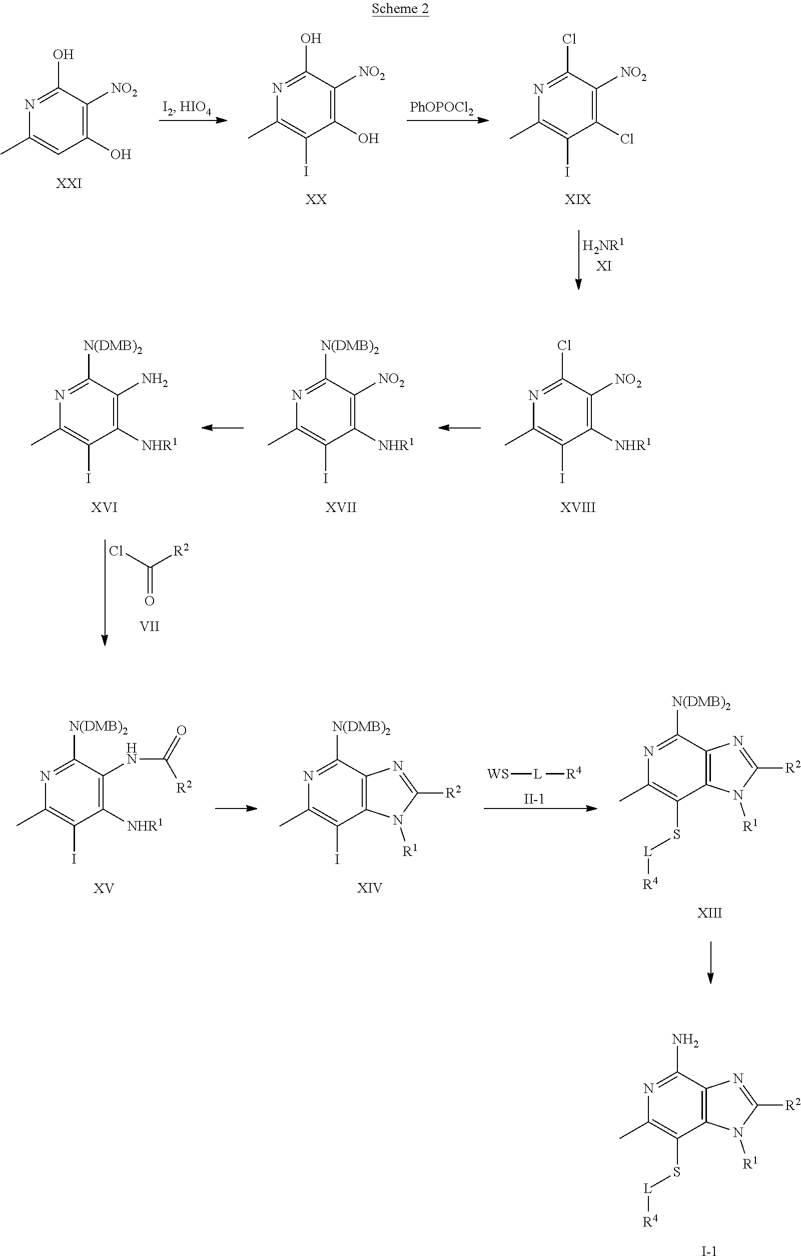

wherein W is H, —S-L-R⁴ or R^{a}; R^{a} is alkoxycarbonylalkyl.

The compounds of formula (I-1) can also be prepared according to Scheme 2.

Iodization of a compound of formula (XXI) affords a compound of formula (XX) in the presence of a suitable reagent such as I₂ with HIO₄. A compound of formula (XIX) is prepared by treating a compound of formula (XX) with phenyl dichlorophosphate. A compound of formula (XVIII) is prepared by reaction of a compound of formula (XIX) and a compound of formula (XI) in the presence of a suitable base, such as NH₃ or DIEA. A compound of formula (XVIII) is converted to a compound of formula (XVII) by introduction of 2,4-dimethoxyphenyl)methyl protecting group. A compound of formula (XVII) can be reduced by a reducing agent, such as Pd/C with H₂, Pd(OH)₂ with H₂, or Fe with NH₄Cl to give a compound of formula (XVI). Condensation of a compound of formula (XVI) with a compound of formula (VII) and a suitable base, such as DIEA and NEt₃, affords a compound of formula (XV). A compound of formula (XIV) is prepared by reaction of a compound of formula (XV) in the presence of a suitable base, such as NaOH.

A compound of formula (I-1) can be prepared by coupling of a compound of formula (XIV) and a compound of formula (II-1) in the presence of a suitable reagent such as Cu₂S, Fe and a suitable base such as K₂CO₃ and Cs₂CO₃, followed by deprotection in the presence of a suitable acid, such as TFA and HCl.

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising any one of the following steps:
a) the formation of compound of formula (I-1),

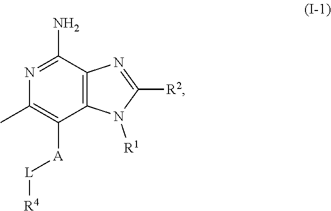

via reaction between compound of formula (III),

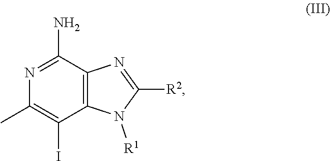

and compound of formula (II-1), WS-L-R⁴ (II-1), in the presence of a set of catalyst, ligand, reagent and base;
b) the formation of compound of formula (I-1) via deprotection of compound of formula (XIII),

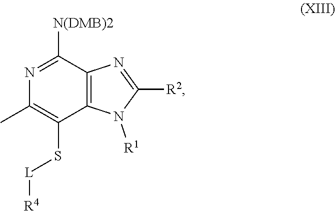

in the presence of an acid;
c) the formation of compound of formula (1-2),

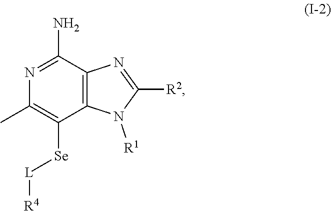

via the reaction between compound of formula (III) and compound of formula (II-2), B(OH)₂-L-R⁴ (II-2), in the presence of reagents;
wherein the set of catalyst, ligand, reagent and base in step a) can be, for example (1) Pd₂(dba)₃, XantPhos and DIEA (or Cs₂CO₃), (2) CuI, Mtpy and KOH, (3) Cu₂S, Fe and K₂CO₃ (or Cs₂CO₃), or (4) TIPSSH, XantPhos Pd G3 and K₂CO₃ (or Cs₂CO₃);
the acid in step b) can be, for example TFA or HCl;
the reagents in step c) can be, for example, SeO₂ and KI.

A compound of formula (I) when manufactured according to the above process is also an object of the invention.

### EXAMPLES

The invention will be more fully understood by reference to the following examples. They should not, however, be construed as limiting the scope of the invention.

### Abbreviations

aq.: aqueous
BINAP: 1,1′-binaphthyl-2,2′-diphenyl phosphine
DABCO: 1,4-diazabicyclo[2.2.2]octane
DDQ: 2,3-dichloro-5,6-dicyano-1,4-benzoquinone
DIEA: N,N-diethylpropylamine
DMAP: 4-dimethylaminopyridine
DMB: 2,4-dimethoxybenzyl
DMF: dimethyl formamide
EC₅₀: the molar concentration of an agonist, which produces 50% of the
maximum possible response for that agonist.
eq: equivalent
EtOAc or EA: ethyl acetate
FA: formic acid
h or hr(s): hour(s)
HPLC: high performance liquid chromatography
LDA: lithium diisopropylamide
MS (ESI): mass spectroscopy (electron spray ionization)
Mtpy: 4′-(4-methoxyphenyl)-2,2′:6,2″-terpyridine
NEt₃: triethylamine
NIS: N-iodosuccinimide
NMR: nuclear magnetic resonance
obsd.: observed
Pd₂(allyl)₂Cl₂: allylpalladium(II) chloride dimer
Pd₂(dba)₃: tris(dibenzylideneacetone)dipalladium
PE: petroleum ether
prep-HPLC: preparative high performance liquid chromatography
RT or rt: room temperature
sat.: saturated
SFC: supercritical fluid chromatography
TFA: trifluoroacetic acid
THF: tetrahydrofuran
TIPSSH: triisopropylsilanethiol
TLC: thin layer chromatography
V/V: volume ratio
XantPhos: (9,9-dimethyl-9H-xanthene-4,5-diyl)bis(diphenylphosphine)
XantPhos Pd G3: [2′-(amino-κN)[1,1′-biphenyl]-2-yl-κC][[5-(diphenylphosphino)-9,9-dimethyl-9H-xanthen-4-yl]diphenylphosphine-κP](methanesulfonato-κO)palladium
XPhos Pd G4: (SP-4-3)-[dicyclohexyl[2′,4′,6′-tris(1-methylethyl)[1,1′-biphenyl]-2-yl]phosphine](methanesulfonato-κO)[2′-(methylamino-κN)[1,1′-biphenyl]-2-yl-κC]palladium

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using Phenomenex Luna C18 (15 μm, 150×400 mm) column, Phenomenex Luna C18 (10 μm, 100×250 mm) column, Phenomenex Luna C18 (10 μm, 80×250 mm) column, Shim-pack C18 (10 μm, 25×150 mm) column, Phenomenex Synergi C18 (10 μm, 25×150 mm) column, or Waters Atlantis T3 (5 μm, 30×150 mm) column. Waters AutoP purification System (Sample Manager 2767, Pump 2525, Detector: Micromass ZQ and UV 2487, solvent system: acetonitrile and 0.1% ammonium bicarbonate in water; acetonitrile and 0.1% FA in water or acetonitrile and 0.1% TFA in water). Or Gilson-281 purification System (Pump 322, Detector: UV 156, solvent system: acetonitrile and 0.05% ammonium bicarbonate in water; acetonitrile and 0.225% FA in water; acetonitrile and 0.05% HCl in water; acetonitrile and 0.075% TFA in water; or acetonitrile and water).

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz or Bruker Avance 500 MHz.

All reactions involving air-sensitive reagents were performed under an argon atmosphere.

Reagents were used as received from commercial suppliers without further purification unless otherwise noted.

### PREPARATIVE EXAMPLES

### Example 1

### 7-[4-[(dimethylamino)methyl]phenyl]sulfanyl-2-(ethoxymethyl)-6-methyl-1H-imidazo[4,5-c]pyridin-4-amine

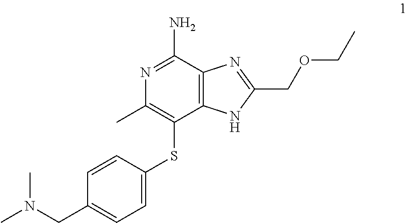 [SMILES: CCOCc1nc2c(N)nc(C)c(Sc3ccc(CN(C)C)cc3)c2[nH]1]

### Step 1: Preparation of 2-chloro-6-methyl-3-nitro-pyridin-4-amine (Compound 1a)

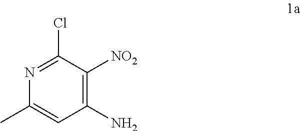

To a solution of 2,4-dichloro-6-methyl-3-nitro-pyridine (30.0 g, 144.92 mmol) in THF (300 mL) was added NH₃/MeOH (120.0 mL, 840.0 mmol, 7.0 M) at 20° C. The mixture was stirred at 50° C. for 16 h. Then the mixture was poured into H₂O (200 mL) and extracted with EA (3×500 mL). The organic layer was washed with brine (3×200 mL), dried over anhydrous Na₂SO₄, filtered and concentrated in vacuo. The residue was purified by column to afford 2-chloro-6-methyl-3-nitro-pyridin-4-amine (8.3 g, Compound 1a) as a light yellow solid. MS obsd. (ESI⁺) [(M+H)⁺]: 188.0.

### Step 2: Preparation of N2,N2-bis[(2,4-dimethoxyphenyl)methyl]-6-methyl-3-nitro-pyridine-2,4-diamine (Compound 1b)

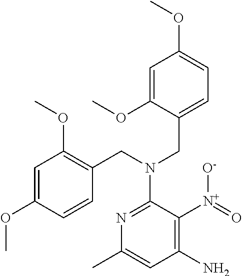 [SMILES: COc1ccc(CN(Cc2ccc(OC)cc2OC)c2nc(C)cc(N)c2[N+](=O)[O-])c(OC)c1]

To a mixture of 2-chloro-6-methyl-3-nitro-pyridin-4-amine (8.3 g, Compound 1a, 44.25 mmol), DIEA (19.66 mL, 110.62 mmol) in THF (120 mL) was added 1-(2,4-dimethoxyphenyl)-N-[(2,4-dimethoxyphenyl)methyl]methanamine (15.45 g, 48.67 mmol) at 20° C. The mixture was stirred at 60° C. for 16 h. Then the reaction mixture was poured into H₂O (200 mL) and extracted with EA (3×200 mL). The combined organic layer was washed with brine (3×200 mL), dried over Na₂SO₄, filtered and concentrated in vacuo. The residue was purified by silica gel column to afford N2,N2-bis[(2,4-dimethoxyphenyl)methyl]-6-methyl-3-nitro-pyridine-2,4-diamine (20.0 g, Compound 1b) as a light yellow oil. MS obsd. (ESI⁺) [(M+H)⁺]: 469.2.

### Step 3: Preparation of N2,N2-bis[(2,4-dimethoxyphenyl)methyl]-6-methyl-pyridine-2,3,4-triamine (Compound 1c)

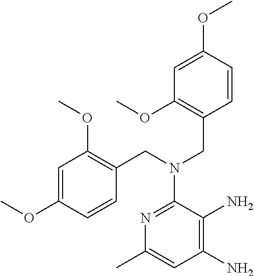

To a solution of N2,N2-bis[(2,4-dimethoxyphenyl)methyl]-6-methyl-3-nitro-pyridine-2,4-diamine (15.0 g, Compound 1b, 32.02 mmol) in methanol (200 mL) was added Pd/C (4.00 g, 10% loaded on active carbon) under N₂. The reaction mixture was stirred at 25° C. for 2 h under H₂ balloon (15 psi). The reaction mixture was filtered through a pad of celite and the residue was washed with MeOH (500 mL). The filtrate was concentrated in vacuo to afford N2,N2-bis[(2,4-dimethoxyphenyl)methyl]-6-methyl-pyridine-2,3,4-triamine (10.0 g, Compound 1c) as a light yellow oil, which was used for next step directly without further purification.

### Step 4: Preparation of N-[4-amino-2-[bis[(2,4-dimethoxyphenyl)methyl]amino]-6-methyl-3-pyridyl]-2-ethoxy-acetamide (Compound 1d)

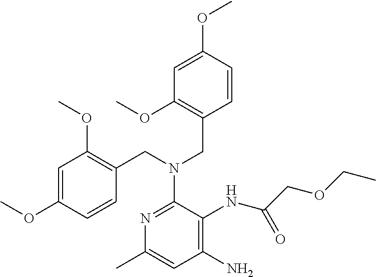 [SMILES: CCOCC(=O)Nc1c(N)cc(C)nc1N(Cc1ccc(OC)cc1OC)Cc1ccc(OC)cc1OC]

To a mixture of N2,N2-bis[(2,4-dimethoxyphenyl)methyl]-6-methyl-pyridine-2,3,4-triamine (10.0 g, Compound 1c, 22.8 mmol) and DIEA (8.12 mL, 45.61 mmol) in DCM (100 mL) was added 2-ethoxyacetyl chloride (2.79 g, 22.8 mmol) at 0° C. The mixture was stirred at 15° C. for 12 h. Then the mixture was diluted with H₂O (200 mL) and extracted with DCM (2×200 mL). The organic layer was washed with brine (200 mL), dried over Na₂SO₄ and filtered. The filtrate was concentrated in vacuo to afford N-[4-amino-2-[bis[(2,4-dimethoxyphenyl)methyl]amino]-6-methyl-3-pyridyl]-2-ethoxy-acetamide (9.0 g, Compound 1d) as a light yellow oil.

### Step 5: Preparation of N,N-bis[(2,4-dimethoxyphenyl)methyl]-2-(ethoxymethyl)-6-methyl-1H-imidazo[4,5-c]pyridin-4-amine (Compound 1e)

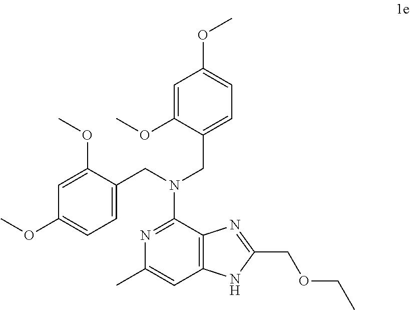 [SMILES: CCOCc1nc2c(N(Cc3ccc(OC)cc3OC)Cc3ccc(OC)cc3OC)nc(C)cc2[nH]1]

To a solution of N-[4-amino-2-[bis[(2,4-dimethoxyphenyl)methyl]amino]-6-methyl-3-pyridyl]-2-ethoxy-acetamide (9.0 g, Compound 1d, 17.16 mmol) in ethanol (100 mL) was added NaOH (100.0 mL, 400.0 mmol, 4 M in H₂O) at 20° C. The reaction mixture was stirred at 80° C. for 12 h. The reaction mixture was concentrated in vacuo to remove EtOH. Then the residue was diluted with H₂O (200 mL) and extracted with EA (3×300 mL). The organic layer was washed with brine (200 mL), dried over Na₂SO₄ and filtered. The filtrate was concentrated in vacuo and the residue was purified by trituration from MeOH (50 mL) to afford N,N-bis[(2,4-dimethoxyphenyl)methyl]-2-(ethoxymethyl)-6-methyl-1H-imidazo[4,5-c]pyridin-4-amine (6.5 g, Compound 1e) as a light yellow solid. MS obsd. (ESI⁺) [(M+H)⁺]: 507.3.

### Step 6: Preparation of 2-(ethoxymethyl)-6-methyl-1H-imidazo[4,5-c]pyridin-4-amine (Compound 1f)

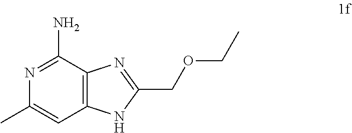

A solution of N,N-bis[(2,4-dimethoxyphenyl)methyl]-2-(ethoxymethyl)-6-methyl-1H-imidazo[4,5-c]pyridin-4-amine (3.0 g, Compound 1e, 5.92 mmol) in DCM (30.0 mL) and TFA (30.0 mL) was stirred at 25° C. for 12 h. The mixture was concentrated in vacuo. Saturated NaHCO₃ (aq.) was added to bring pH to 7. After lyophilisation, the residue was washed with DCM/MeOH (150 mL, V/V=10/1) and filtered. The filtrate was concentrated in vacuo to afford 2-(ethoxymethyl)-6-methyl-1H-imidazo[4,5-c]pyridin-4-amine (1.0 g, Compound 1f) as a yellow oil, which was used for next step directly without further purification.

### Step 7: Preparation of 2-(ethoxymethyl)-7-iodo-6-methyl-1H-imidazo[4,5-c]pyridin-4-amine (Compound 1g)

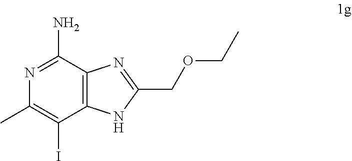 [SMILES: CCOCc1nc2c(N)nc(C)c(I)c2[nH]1]

To a solution of 2-(ethoxymethyl)-6-methyl-1H-imidazo[4,5-c]pyridin-4-amine (1.22 g, Compound 1f, 5.92 mmol) in acetic acid (12.0 mL) was added NIS (1.99 g, 8.87 mmol) at 20° C. The mixture was stirred at 20° C. for 0.5 h. The reaction mixture was concentrated in vacuo. The residue was purified by reversed prep-HPLC to afford 2-(ethoxymethyl)-7-iodo-6-methyl-1H-imidazo[4,5-c]pyridin-4-amine (1.96 g, Compound 1g) as a light yellow solid. MS obsd. (ESI⁺) [(M+H)⁺]: 333.1.

### Step 8: Preparation of O-(4-formylphenyl) N,N-dimethylcarbamothioate (Compound 1h)

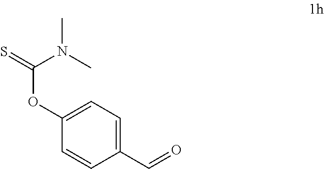

To a solution of 4-hydroxybenzaldehyde (5.0 g, 40.94 mmol) in DMF (80 mL) were added DABCO (18.37 g, 163.77 mmol) and dimethylthiocarbamoyl chloride (10.12 g, 81.89 mmol) at 20° C. The mixture was stirred at 20° C. for 12 h. The mixture was diluted with H₂O (100 mL) and extracted with EA (3×100 mL). The organic layer was washed with brine (3×100 mL), dried over Na₂SO₄, filtered and concentrated in vacuo. The residue was purified by re-crystallization in MeOH (20 mL) to afford O-(4-formylphenyl) N,N-dimethylcarbamothioate (5.0 g, Compound 1h) as a white solid. MS obsd. (ESI⁺) [(M+H)⁺]: 210.1. ¹H NMR (400 MHz, CDCl₃) δ ppm: 10.00 (s, 1H), 7.93 (d, J=8.4 Hz, 2H), 7.24 (d, J=8.4 Hz, 2H), 3.46 (s, 3H), 3.37 (s, 3H).

### Step 9: Preparation of S-(4-formylphenyl) N,N-dimethylcarbamothioate (Compound 1i)

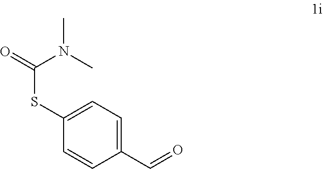

A solution of O-(4-formylphenyl) N,N-dimethylcarbamothioate (2.0 g, Compound 1h, 9.56 mmol) in Ph₂O (30.0 mL) was stirred at 220° C. for 0.5 h. The mixture was purified by silica gel column to afford S-(4-formylphenyl) N,N-dimethylcarbamothioate (1.5 g, Compound 1i) as a white solid. ¹H NMR (400 MHz, CDCl₃) δ ppm: 9.96 (s, 1H), 7.81 (d, J=8.4 Hz, 2H), 7.61 (d, J=8.4 Hz, 2H), 3.04 (br s, 3H), 2.98 (br s, 3H).

### Step 10: Preparation of S-[4-[(dimethylamino)methyl]phenyl]N,N-dimethylcarbamothioate (Compound 1j)

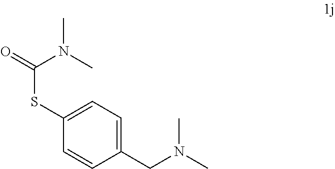 [SMILES: CN(C)Cc1ccc(SC(=O)N(C)C)cc1]

A mixture of S-(4-formylphenyl) N,N-dimethylcarbamothioate (500.0 mg, Compound 1i, 2.39 mmol), dimethylamine hydrochloride (253.28 mg, 3.11 mmol) and acetic acid (43.04 mg, 0.72 mmol) in DCM (8.0 mL) was stirred at 20° C. for 1 h. Then to the mixture was added NaBH(OAc)₃ (1.01 g, 4.78 mmol) at 20° C. The mixture was stirred at 20° C. for 2 h, then concentrated in vacuo. The residue was purified by silica gel column to afford S-[4-[(dimethylamino)methyl]phenyl]N,N-dimethylcarbamothioate (450.0 mg, Compound 1j) as a light yellow oil. MS obsd. (ESI⁺) [(M+H)⁺]: 239.2.

### Step 11: Preparation of 4-[(dimethylamino)methyl]benzenethiol (Compound 1k)

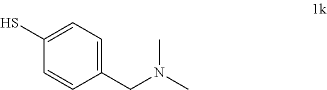

A mixture of S-[4-[(dimethylamino)methyl]phenyl]N,N-dimethylcarbamothioate (440.0 mg, Compound 1j, 1.85 mmol), and KOH (1.03 g, 18.46 mmol) in methanol (10.0 mL) and water (4.0 mL) was stirred at 80° C. for 2 h. The pH of the mixture was adjusted to 7 by sat. HCl (3.0 M aq.), then the mixture was concentrated in vacuo. The residue was purified by prep-HPLC to afford 4-[(dimethylamino)methyl]benzenethiol (200.0 mg, Compound 1k) as a white solid. MS obsd. (ESI⁺) [(M+H)⁺]: 168.0.

### Step 12: Preparation of 7-[4-[(dimethylamino)methyl]phenyl]sulfanyl-2-(ethoxymethyl)-6-methyl-1H-imidazo[4,5-c]pyridin-4-amine (Example 1)

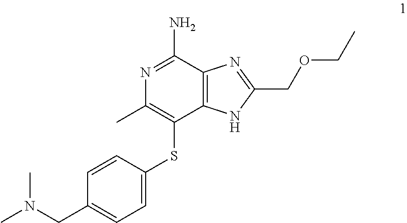 [SMILES: CCOCc1nc2c(N)nc(C)c(Sc3ccc(CN(C)C)cc3)c2[nH]1]

To a mixture of 4-[(dimethylamino)methyl]benzenethiol (29.46 mg, Compound 1k, 0.18 mmol) and 2-(ethoxymethyl)-7-iodo-6-methyl-1H-imidazo[4,5-c]pyridin-4-amine (50.0 mg, Compound 1g, 0.15 mmol) in 1,4-dioxane (2.0 mL) were added DIEA (0.05 mL, 0.3 mmol), Pd₂(dba)₃ (27.54 mg, 0.03 mmol) and XantPhos (17.42 mg, 0.03 mmol) at 20° C. The suspension was degassed in vacuo and purged with N₂ three times. The suspension was stirred at 100° C. for 4 h under N₂. The reaction mixture was filtered through celite. The filtrate was concentrated in vacuo, and the residue was purified by prep-HPLC to afford 7-[4-[(dimethylamino)methyl]phenyl]sulfanyl-2-(ethoxymethyl)-6-methyl-1H-imidazo[4,5-c]pyridin-4-amine (20.9 mg, Example 1) as a white solid. MS obsd. (ESI⁺) [(M+H)⁺]: 372.1. ¹H NMR (400 MHz, CD₃OD) δ ppm: 7.41 (d, J=8.0 Hz, 2H), 7.21 (d, J=8.0 Hz, 2H), 4.67 (s, 2H), 4.25 (s, 2H), 3.65-3.57 (m, 2H), 2.82 (s, 6H), 2.67 (s, 3H), 1.20 (t, J=7.2 Hz, 3H).

### Example 2

### 1-[4-amino-2-butyl-7-[4-[(dimethylamino)methyl]phenyl]sulfanyl-6-methyl-imidazo[4,5-c]pyridin-1-yl]-2-methyl-propan-2-ol

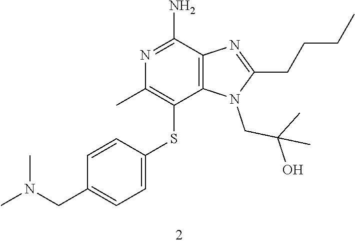 [SMILES: CCCCc1nc2c(N)nc(C)c(Sc3ccc(CN(C)C)cc3)c2n1CC(C)(C)O]

### Step 1: Preparation of 1-[(2-chloro-6-methyl-3-nitro-4-pyridyl)amino]-2-methyl-propan-2-ol (Compound 2a)

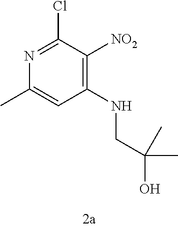 [SMILES: Cc1cc(NCC(C)(C)O)c([N+](=O)[O-])c(Cl)n1]

To a mixture of 2,4-dichloro-6-methyl-3-nitro-pyridine (17.0 g, 82.12 mmol) and DIEA (21.22 g, 164.24 mmol) in THF (200 mL) was added 1-amino-2-methylpropan-2-ol (7.32 g, 82.12 mmol) dropwise at 0° C. The mixture was stirred at 15° C. for 16 h, and then poured into sat. NH₄Cl (300 mL), extracted with EA (3×200 mL), and the organic layer was washed with brine (3×200 mL), dried over anhydrous Na₂SO₄, filtered and concentrated in vacuo. The residue was purified by silica gel column to afford 1-[(2-chloro-6-methyl-3-nitro-4-pyridyl)amino]-2-methyl-propan-2-ol (7.00 g, Compound 2a) as a yellow solid. ¹H NMR (400 MHz, CDCl₃) a ppm: 7.03 (br s, 1H), 6.52 (s, 1H), 3.22 (d, J=5.2 Hz, 2H), 2.43 (s, 3H), 1.35 (s, 6H).

### Step 2: Preparation of 1-[[2-[bis[(2,4-dimethoxyphenyl)methyl]amino]-6-methyl-3-nitro-4-pyridyl]amino]-2-methyl-propan-2-ol (Compound 2b)

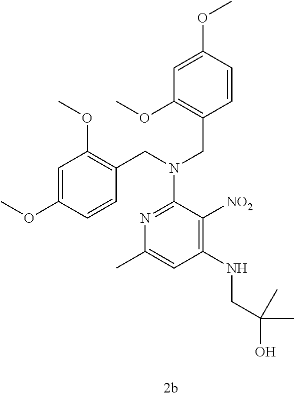 [SMILES: COc1ccc(CN(Cc2ccc(OC)cc2OC)c2nc(C)cc(NCC(C)(C)O)c2[N+](=O)[O-])c(OC)c1]

A mixture of 1-[(2-chloro-6-methyl-3-nitro-4-pyridyl)amino]-2-methyl-propan-2-ol (7.00 g, Compound 2a, 26.96 mmol), 1-(2,4-dimethoxyphenyl)-N-[(2,4-dimethoxyphenyl)methyl]methanamine (10.26 g, 32.35 mmol) and DIEA (6.96 g, 53.91 mmol) in DMF (100 mL) was stirred at 60° C. for 12 h. The mixture was poured into H₂O (200 mL) and extracted with EA (3×200 mL). The organic layer was washed with brine (3×200 mL), dried over Na₂SO₄, filtered and concentrated in vacuo. The residue was purified by silica gel column to afford 1-[[2-[bis[(2,4-dimethoxyphenyl)methyl]amino]-6-methyl-3-nitro-4-pyridyl]amino]-2-methyl-propan-2-ol (12.0 g, Compound 2b) as a yellow oil.

### Step 3: Preparation of 1-[[3-amino-2-[bis[(2,4-dimethoxyphenyl)methyl]amino]-6-methyl-4-pyridyl]amino]-2-methyl-propan-2-ol (Compound 2c)

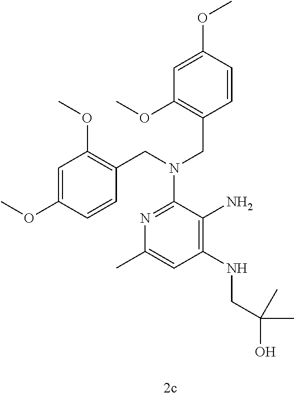 [SMILES: COc1ccc(CN(Cc2ccc(OC)cc2OC)c2nc(C)cc(NCC(C)(C)O)c2N)c(OC)c1]

To a solution of 1-[[2-[bis[(2,4-dimethoxyphenyl)methyl]amino]-6-methyl-3-nitro-4-pyridyl]amino]-2-methyl-propan-2-ol (12.0 g, Compound 2b, 22.2 mmol) in methanol (500 mL) was added Pd(OH)₂/C (2.40 g, 10% loaded on active carbon) under N₂ at 20° C. The mixture was degassed in vacuo and purged with H₂ three times. Then the mixture was stirred at 20° C. for 0.5 h under H₂ balloon (15 Psi). The mixture was filtered through celite and washed with MeOH (800 mL). The filtrate was concentrated in vacuo to afford 1-[[3-amino-2-[bis[(2,4-dimethoxyphenyl)methyl]amino]-6-methyl-4-pyridyl]amino]-2-methyl-propan-2-ol (10.0 g, Compound 2c) as a brown oil. MS obsd. (ESI⁺) [(M+H)⁺]: 511.3.

### Step 4: Preparation of N-[2-[bis[(2,4-dimethoxyphenyl)methyl]amino]-4-[(2-hydroxy-2-methyl-propyl)amino]-6-methyl-3-pyridyl]pentanamide (Compound 2d)

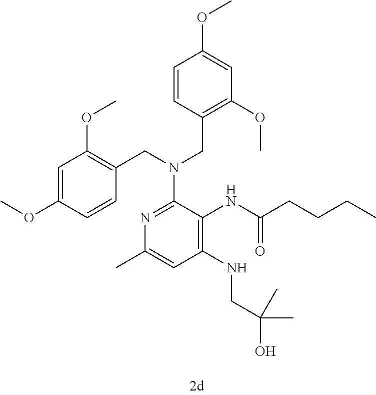 [SMILES: CCCCC(=O)Nc1c(NCC(C)(C)O)cc(C)nc1N(Cc1ccc(OC)cc1OC)Cc1ccc(OC)cc1OC]

Compound 2d was prepared in analogy to Example 1, Step 4 by using 1-[[3-amino-2-[bis[(2,4-dimethoxyphenyl)methyl]amino]-6-methyl-4-pyridyl]amino]-2-methyl-propan-2-ol (Compound 2c) instead of N2,N2-bis[(2,4-dimethoxyphenyl)methyl]-6-methyl-pyridine-2,3,4-triamine (Compound 1c) and valeryl chloride instead of 2-ethoxyacetyl chloride. N-[2-[bis[(2,4-dimethoxyphenyl)methyl]amino]-4-[(2-hydroxy-2-methyl-propyl)amino]-6-methyl-3-pyridyl]pentanamide (4.7 g, Compound 2d) was obtained as a black oil. MS obsd. (ESI⁺) [(M+H)⁺]: 595.3.

### Step 5: Preparation of 1-[4-[bis[(2,4-dimethoxyphenyl)methyl]amino]-2-butyl-6-methyl-imidazo[4,5-c]pyridin-1-yl]-2-methyl-propan-2-ol (Compound 2e)

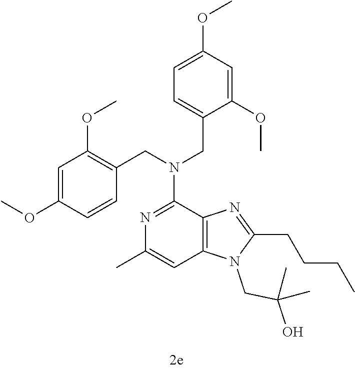 [SMILES: CCCCc1nc2c(N(Cc3ccc(OC)cc3OC)Cc3ccc(OC)cc3OC)nc(C)cc2n1CC(C)(C)O]

To a solution of N-[2-[bis[(2,4-dimethoxyphenyl)methyl]amino]-4-[(2-hydroxy-2-methyl-propyl)amino]-6-methyl-3-pyridyl]pentanamide (4.7 g, Compound 2d, 7.9 mmol) in ethanol (30 mL) was added aq. NaOH (30.0 mL, 79.03 mmol) at 20° C. The reaction mixture was stirred at 80° C. for 0.5 h, and then extracted with EA (3×150 mL). The combined organic layer was washed with brine (3×100 mL), dried with Na₂SO₄, filtered and concentrated in vacuo. The residue was purified by silica gel column to afford 1-[4-[bis[(2,4-dimethoxyphenyl)methyl]amino]-2-butyl-6-methyl-imidazo[4,5-c]pyridin-1-yl]-2-methyl-propan-2-ol (3.2 g, Compound 2e) as a yellow oil. MS obsd. (ESI⁺) [(M+H)⁺]: 577.4.

### Step 6: Preparation of 1-(4-amino-2-butyl-6-methyl-imidazo[4,5-c]pyridin-1-yl)-2-methyl-propan-2-ol (Compound 2f)

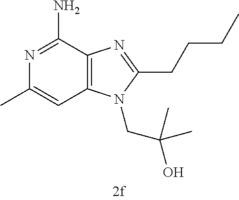 [SMILES: CCCCc1nc2c(N)nc(C)cc2n1CC(C)(C)O]

A solution of 1-[4-[bis[(2,4-dimethoxyphenyl)methyl]amino]-2-butyl-6-methyl-imidazo[4,5-c]pyridin-1-yl]-2-methyl-propan-2-ol (2.2 g, Compound 2e, 3.81 mmol) and TFA (20.0 mL) in DCM (20.0 mL) was stirred at 20° C. for 1 h. The reaction mixture was concentrated in vacuo and the residue was purified by reversed prep-HPLC to afford 1-(4-amino-2-butyl-6-methyl-imidazo[4,5-c]pyridin-1-yl)-2-methyl-propan-2-ol (1.48 g, Compound 2f) as a purple oil, which was used for next step directly without further purification. MS obsd. (ESI⁺) [(M+H)⁺]: 277.1.

### Step 7: Preparation of 1-(4-amino-2-butyl-7-iodo-6-methyl-imidazo[4,5-c]pyridin-1-yl)-2-methyl-propan-2-ol (Compound 2g)

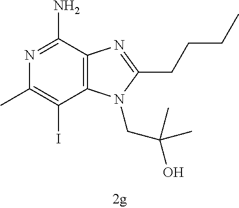 [SMILES: CCCCc1nc2c(N)nc(C)c(I)c2n1CC(C)(C)O]

Compound 2g was prepared in analogy to Example 1, Step 7 by using 1-(4-amino-2-butyl-6-methyl-imidazo[4,5-c]pyridin-1-yl)-2-methyl-propan-2-ol (Compound 2f) instead of 2-(ethoxymethyl)-6-methyl-1H-imidazo[4,5-c]pyridin-4-amine (Compound 1f). 1-(4-Amino-2-butyl-7-iodo-6-methyl-imidazo[4,5-c]pyridin-1-yl)-2-methyl-propan-2-ol (900.0 mg, Compound 2g) was obtained as a yellow solid. MS obsd. (ESI⁺) [(M+H)⁺]: 403.0.

### Step 8: Preparation of 1-[4-amino-2-butyl-7-[4-[(dimethylamino)methyl]phenyl]sulfanyl-6-methyl-imidazo[4,5-c]pyridin-1-yl]-2-methyl-propan-2-ol (Example 2)

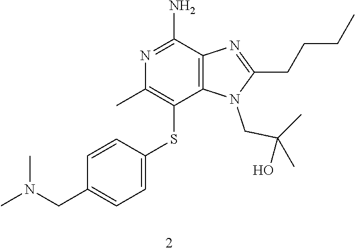 [SMILES: CCCCc1nc2c(N)nc(C)c(Sc3ccc(CN(C)C)cc3)c2n1CC(C)(C)O]

To a mixture of 1-(4-amino-2-butyl-7-iodo-6-methyl-imidazo[4,5-c]pyridin-1-yl)-2-methyl-propan-2-ol (150.0 mg, Compound 2g, 0.37 mmol), 4-[(dimethylamino)methyl]benzenethiol (207.91 mg, Compound 1k, 1.24 mmol) and DIEA (321.2 mg, 2.49 mmol) in 1,4-dioxane (3.0 mL) were added XantPhos (143.84 mg, 0.25 mmol) and Pd₂(dba)₃ (227.4 mg, 0.25 mmol) at 20° C. The suspension was degassed in vacuo and purged with N₂ three times. The mixture was stirred at 100° C. for 16 h under N₂ atmosphere, and then concentrated in vacuo. The residue was purified by prep-HPLC to afford 1-[4-amino-2-butyl-7-[4-[(dimethylamino)methyl]phenyl]sulfanyl-6-methyl-imidazo[4,5-c]pyridin-1-yl]-2-methyl-propan-2-ol (7.8 mg, Example 2) as a white solid. MS obsd. (ESI⁺) [(M+H)⁺]: 442.4. ¹H NMR (400 MHz, CD₃OD) δ ppm: 7.19 (d, J=8.4 Hz, 2H), 6.85 (d, J=8.4 Hz, 2H), 3.45 (s, 2H), 3.37-3.32 (m, 2H), 3.05 (t, J=7.6 Hz, 2H), 2.54 (s, 3H), 2.25 (s, 6H), 1.83-1.74 (m, 2H), 1.47-1.38 (m, 2H), 1.15 (br s, 6H), 0.96 (t, J=7.2 Hz, 3H).

### Example 3

### 1-[4-amino-7-[4-[(dimethylamino)methyl]phenyl]selanyl-2-(ethoxymethyl)-6-methyl-imidazo[4,5-c]pyridin-1-yl]-2-methyl-propan-2-ol

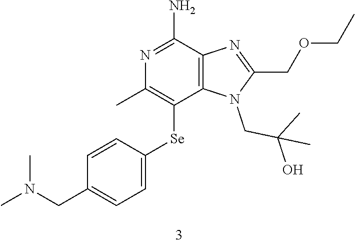 [SMILES: CCOCc1nc2c(N)nc(C)c([Se]c3ccc(CN(C)C)cc3)c2n1CC(C)(C)O]

### Step 1: Preparation of N-[2-[bis[(2,4-dimethoxyphenyl)methyl]amino]-4-[(2-hydroxy-2-methyl-propyl)amino]-6-methyl-3-pyridyl]-2-ethoxy-acetamide (Compound 3a)

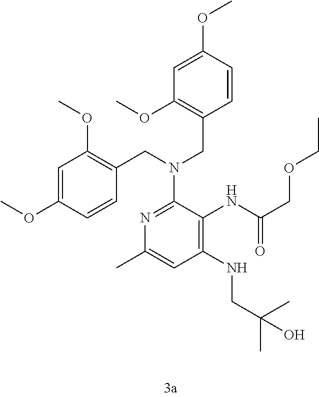 [SMILES: CCOCC(=O)Nc1c(NCC(C)(C)O)cc(C)nc1N(Cc1ccc(OC)cc1OC)Cc1ccc(OC)cc1OC]

Compound 3a was prepared in analogy to Example 1, Step 4 by using 1-[[3-amino-2-[bis[(2,4-dimethoxyphenyl)methyl]amino]-6-methyl-4-pyridyl]amino]-2-methyl-propan-2-ol (Compound 2c) instead of N2,N2-bis[(2,4-dimethoxyphenyl)methyl]-6-methyl-pyridine-2,3,4-triamine (Compound 1c). N-[2-[bis[(2,4-dimethoxyphenyl)methyl]amino]-4-[(2-hydroxy-2-methyl-propyl)amino]-6-methyl-3-pyridyl]-2-ethoxy-acetamide (11.0 g, Compound 3a) was obtained as a brown oil.

### Step 2: Preparation of 1-[4-[bis[(2,4-dimethoxyphenyl)methyl]amino]-2-(ethoxymethyl)-6-methyl-imidazo[4,5-c]pyridin-1-yl]-2-methyl-propan-2-ol (Compound 3b)

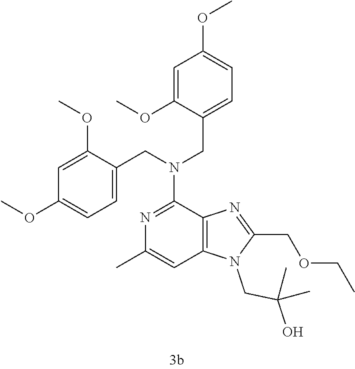 [SMILES: CCOCc1nc2c(N(Cc3ccc(OC)cc3OC)Cc3ccc(OC)cc3OC)nc(C)cc2n1CC(C)(C)O]

To a solution of N-[2-[bis[(2,4-dimethoxyphenyl)methyl]amino]-4-[(2-hydroxy-2-methyl-propyl)amino]-6-methyl-3-pyridyl]-2-ethoxy-acetamide (11.0 g, Compound 3a, 18.43 mmol) in ethanol (120 mL) and water (40 mL) was added NaOH (1.49 g, 36.87 mmol) at 20° C. The mixture was stirred at 80° C. for 2 h. Then, the reaction mixture was concentrated in vacuo. The residue was diluted with H₂O (200 mL) and extracted with EA (3×200 mL). The organic layer was washed with brine (200 mL), dried over Na₂SO₄, filtered and concentrated in vacuo. The residue was purified by silica gel column to afford 1-[4-[bis[(2,4-dimethoxyphenyl)methyl]amino]-2-(ethoxymethyl)-6-methyl-imidazo[4,5-c]pyridin-1-yl]-2-methyl-propan-2-ol (8.00 g, Compound 3b) as a yellow oil. ¹H NMR (400 MHz, CDCl₃) δ ppm: 7.18 (d, J=8.4 Hz, 2H), 6.43 (d, J=2.0 Hz, 2H), 6.40 (s, 1H), 6.35 (dd, J=8.4 Hz, 2.0 Hz, 2H), 5.24 (br s, 4H), 4.65 (s, 2H), 4.10 (s, 2H), 3.77 (s, 6H), 3.75 (s, 6H), 3.48-3.39 (m, 2H), 2.42 (s, 3H), 1.27 (s, 6H), 1.12 (t, J=7.2 Hz, 3H).

### Step 3: Preparation of 1-[4-amino-2-(ethoxymethyl)-6-methyl-imidazo[4,5-c]pyridin-1-yl]-2-methyl-propan-2-ol (Compound 3c)

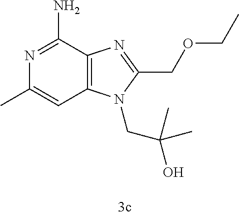 [SMILES: CCOCc1nc2c(N)nc(C)cc2n1CC(C)(C)O]

A solution of 1-[4-[bis[(2,4-dimethoxyphenyl)methyl]amino]-2-(ethoxymethyl)-6-methyl-imidazo[4,5-c]pyridin-1-yl]-2-methyl-propan-2-ol (6.00 g, Compound 3b, 10.37 mmol) and TFA (50 mL) in DCM (50 mL) was stirred at 20° C. for 1 h. The mixture was concentrated in vacuo. The residue was purified by reversed prep-HPLC to afford 1-[4-amino-2-(ethoxymethyl)-6-methyl-imidazo[4,5-c]pyridin-1-yl]-2-methyl-propan-2-ol (2.80 g, Compound 3c) as a yellow solid. MS obsd. (ESI⁺) [(M+H)⁺]: 279.1.

### Step 4: Preparation of 1-[4-amino-7-[4-[(dimethylamino)methyl]phenyl]selanyl-2-(ethoxymethyl)-6-methyl-imidazo[4,5-c]pyridin-1-yl]-2-methyl-propan-2-ol (Example 3)

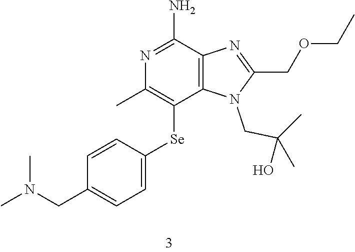 [SMILES: CCOCc1nc2c(N)nc(C)c([Se]c3ccc(CN(C)C)cc3)c2n1CC(C)(C)O]

To a solution of [4-[(dimethylamino)methyl]phenyl]boronic acid (154.36 mg, 0.86 mmol) and 1-[4-amino-2-(ethoxymethyl)-6-methyl-imidazo[4,5-c]pyridin-1-yl]-2-methyl-propan-2-ol (200.0 mg, Compound 3c, 0.72 mmol) in DMSO (3.0 mL) were added KI (119.27 mg, 0.72 mmol) and SeO₂ (95.67 mg, 0.86 mmol) at 20° C. The suspension was degassed in vacuo and purged with N₂ three times. The mixture was stirred at 110° C. for 8 h under N₂ atmosphere. The mixture was filtered through celite and the filtrate was concentrated in vacuo. The residue was purified by prep-HPLC to afford 1-[4-amino-7-[4-[(dimethylamino)methyl]phenyl]selanyl-2-(ethoxymethyl)-6-methyl-imidazo[4,5-c]pyridin-1-yl]-2-methyl-propan-2-ol (10.9 mg, Example 3) as a light yellow solid. MS obsd. (ESI⁺) [(M+H)⁺]: 492.2. ¹H NMR (400 MHz, CD₃OD) δ ppm: 7.15 (d, J=8.4 Hz, 2H), 6.99 (d, J=8.4 Hz, 2H), 4.99-4.93 (m, 2H), 3.56-3.49 (m, 2H), 3.39 (s, 2H), 3.34-3.32 (m, 2H), 2.62 (s, 3H), 2.20 (s, 6H), 1.15 (t, J=8.0 Hz, 3H), 1.14 (s, 6H).

### Example 4

### 7-[4-[(dimethylamino)methyl]-2,6-dimethoxy-phenyl]sulfanyl-2-(ethoxymethyl)-6-methyl-1H-imidazo[4,5-c]pyridin-4-amine

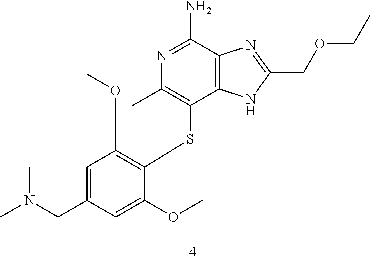 [SMILES: CCOCc1nc2c(N)nc(C)c(Sc3c(OC)cc(CN(C)C)cc3OC)c2[nH]1]

### Step 1: Preparation of O-(4-formyl-2,6-dimethoxy-phenyl) N,N-dimethylcarbamothioate (Compound 4a)

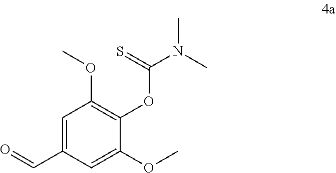 [SMILES: COc1cc(C=O)cc(OC)c1OC(=S)N(C)C]

Compound 4a was prepared in analogy to Example 1, Step 8 by using syringaldehyde instead of 4-hydroxybenzaldehyde. O-(4-Formyl-2,6-dimethoxy-phenyl) N,N-dimethylcarbamothioate (3.98 g, Compound 4a) was obtained as a white solid. MS obsd. (ESI⁺) [(M+H)⁺]: 270.1. ¹H NMR (400 MHz, CDCl₃) δ ppm: 9.92 (s, 1H), 7.18 (s, 2H), 3.91 (s, 6H), 3.47 (s, 3H), 3.38 (s, 3H).

### Step 2: Preparation of S-(4-formyl-2,6-dimethoxy-phenyl) N,N-dimethylcarbamothioate (Compound 4b)

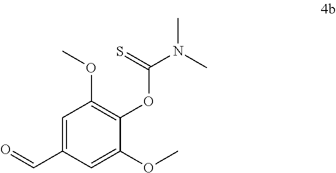 [SMILES: COc1cc(C=O)cc(OC)c1OC(=S)N(C)C]

To a solution of O-(4-formyl-2,6-dimethoxy-phenyl) N,N-dimethylcarbamothioate (3.98 g, Compound 4a, 14.78 mmol) in Ph₂O (60.0 mL) was stirred at 240° C. for 1 h. The mixture was purified by silica gel column to afford S-(4-formyl-2,6-dimethoxy-phenyl) N,N-dimethylcarbamothioate (3.08 g, Compound 4b) as a yellow solid. MS obsd. (ESI⁺) [(M+H)⁺]: 270.1. ¹H NMR (400 MHz, CDCl₃) δ ppm: 9.98 (s, 1H), 7.13 (s, 2H), 3.96 (s, 6H), 3.20 (br s, 3H), 3.01 (br s, 3H).

### Step 3: Preparation of S-[4-[(dimethylamino)methyl]-2,6-dimethoxy-phenyl]N,N-dimethylcarbamothioate (Compound 4c)

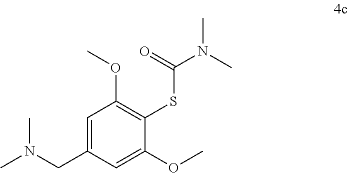

Compound 4c was prepared in analogy to Example 1, Step 10 by using S-(4-formyl-2,6-dimethoxy-phenyl) N,N-dimethylcarbamothioate (Compound 4b) instead of S-(4-formylphenyl) N,N-dimethylcarbamothioate (Compound 1i). S-[4-[(dimethylamino)methyl]-2,6-dimethoxy-phenyl]N,N-dimethylcarbamothioate (1.9 g, Compound 4c) was obtained as a yellow solid. MS obsd. (ESI⁺) [(M+H)⁺]: 299.1.

### Step 4: Preparation of 4-[(dimethylamino)methyl]-2,6-dimethoxy-benzenethiol (Compound 4d)

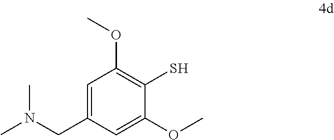

A mixture of S-[4-[(dimethylamino)methyl]-2,6-dimethoxy-phenyl]N,N-dimethylcarbamothioate (1.9 g, Compound 4c, 6.37 mmol) and KOH (1.79 g, 31.84 mmol) in water (5.0 mL) and methanol (15 mL) was stirred at 80° C. for 2 h. The mixture was acidified to pH=6 by aq. HCl (3.0 M) at 0° C. The mixture was concentrated in vacuo. The residue was purified by reversed prep-HPLC to afford 4-[(dimethylamino)methyl]-2,6-dimethoxy-benzenethiol (2.4 g, Compound 4d) as a yellow solid. MS obsd. (EST⁺) [(M+H)⁺]: 228.2.

### Step 5: Preparation of 7-[4-[(dimethylamino)methyl]-2,6-dimethoxy-phenyl]sulfanyl-2-(ethoxymethyl)-6-methyl-1H-imidazo[4,5-c]pyridin-4-amine (Example 4)

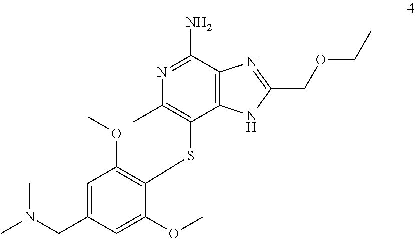 [SMILES: CCOCc1nc2c(N)nc(C)c(Sc3c(OC)cc(CN(C)C)cc3OC)c2[nH]1]

To a mixture of 2-(ethoxymethyl)-7-iodo-6-methyl-1H-imidazo[4,5-c]pyridin-4-amine (100.0 mg, Compound 1g, 0.3 mmol), 4-[(dimethylamino)methyl]-2,6-dimethoxy-benzenethiol (82.13 mg, Compound 4d, 0.36 mmol) and KOH (33.79 mg, 0.6 mmol) in DMSO (3.0 mL) were added CuI (5.73 mg, 0.03 mmol) and Mtpy (10.22 mg, 0.03 mmol) at 20° C. The mixture was stirred at 110° C. for 12 h under N₂. Then the mixture was concentrated in vacuo. The residue was purified by prep-HPLC to afford 7-[4-[(dimethylamino)methyl]-2,6-dimethoxy-phenyl]sulfanyl-2-(ethoxymethyl)-6-methyl-1H-imidazo[4,5-c]pyridin-4-amine (5.5 mg, Example 4) as an off-white solid. MS obsd. (ESI⁺) [(M+H)⁺]: 432.4. ¹H NMR (400 MHz, DMSO-d₆) δ ppm: 11.88 (br s, 1H), 6.58 (s, 2H), 6.26 (br s, 2H), 4.61 (s, 2H), 3.69 (s, 6H), 3.59-3.55 (m, 2H), 3.34 (s, 2H), 2.40 (s, 3H), 2.13 (s, 6H), 1.16 (t, J=6.8 Hz, 3H).

### Example 5

### 2-(ethoxymethyl)-7-[4-[[ethyl(propyl)amino]methyl]phenyl]sulfanyl-6-methyl-1H-imidazo[4,5-c]pyridin-4-amine

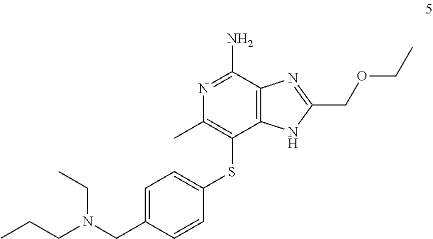 [SMILES: CCCN(CC)Cc1ccc(Sc2c(C)nc(N)c3nc(COCC)[nH]c23)cc1]

Step 1: Preparation of S-[4-[[ethyl(propyl)amino]methyl]phenyl]N,N-dimethylcarbamothioate (Compound 5a)

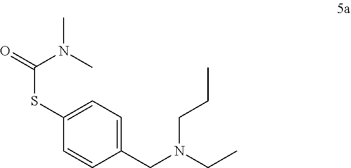 [SMILES: CCCN(CC)Cc1ccc(SC(=O)N(C)C)cc1]

Compound 5a was prepared in analogy to Example 1, Step 10 by using N-ethylpropylamine instead of dimethylamine hydrochloride. S-[4-[[ethyl(propyl)amino]methyl]phenyl]N,N-dimethylcarbamothioate (1.0 g, Compound 5a) was obtained as a light yellow oil. MS obsd. (ESI⁺) [(M+H)⁺]: 281.1.

### Step 2: Preparation of N-ethyl-N-[[4-[[4-[[ethyl(propyl)amino]methyl]phenyl]disulfanyl]phenyl]methyl]propan-1-amine (Compound 5b)

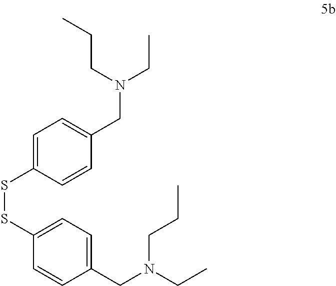 [SMILES: CCCN(CC)Cc1ccc(SSc2ccc(CN(CC)CCC)cc2)cc1]

Compound 5b was prepared in analogy to Example 1, Step 11 by using S-[4-[[ethyl(propyl)amino]methyl]phenyl]N,N-dimethylcarbamothioate (Compound 5a) instead of S-[4-[(dimethylamino)methyl]phenyl]N,N-dimethylcarbamothioate (Compound 1j). N-ethyl-N-[[[4-[[4-[[ethyl(propyl)amino]methyl]phenyl]disulfanyl]phenyl]methyl]propan-1-amine (85.0 mg Compound 5b) was obtained as a grey oil. MS obsd. (ESI⁺) [(M+H)⁺]: 417.4.

### Step 3: Preparation of 2-(ethoxymethyl)-7-[4-[[ethyl(propyl)amino]methyl]phenyl]sulfanyl-6-methyl-1H-imidazo[4,5-c]pyridin-4-amine (Example 5)

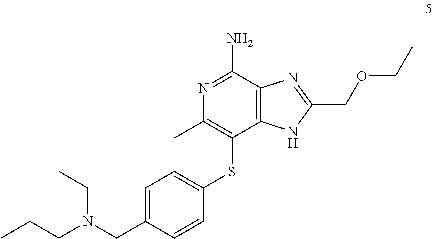 [SMILES: CCCN(CC)Cc1ccc(Sc2c(C)nc(N)c3nc(COCC)[nH]c23)cc1]

To a mixture of 2-(ethoxymethyl)-7-iodo-6-methyl-1H-imidazo[4,5-c]pyridin-4-amine (101.63 mg, Compound 1g, 0.31 mmol), N-ethyl-N-[[4-[[4-[[ethyl(propyl)amino]methyl]phenyl]disulfanyl]phenyl]methyl]propan-1-amine (85.0 mg, Compound 5b, 0.2 mmol) and K₂CO₃ (28.19 mg, 0.2 mmol) in DMSO (2.0 mL) were added Cu₂S (32.46 mg, 0.2 mmol) and Fe powder (6.84 mg, 0.12 mmol) at 20° C. The suspension was degassed in vacuo and purged with N₂ three times. The mixture was stirred at 110° C. for 2 h under N₂ atmosphere. The reaction mixture was filtered. The filtrate was purified by prep-HPLC to afford 2-(ethoxymethyl)-7-[4-[[ethyl(propyl)amino]methyl]phenyl]sulfanyl-6-methyl-1H-imidazo[4,5-c]pyridin-4-amine (7.6 mg, Example 5) as a white solid. MS obsd. (ESI⁺) [(M+H)⁺]: 414.1. ¹H NMR (400 MHz, CD₃OD) δ ppm: 7.17 (d, J=8.4 Hz, 2H), 6.93 (d, J=8.4 Hz, 2H), 4.63 (s, 2H), 3.62-3.54 (m, 2H), 3.50 (s, 2H), 2.52 (s, 3H), 2.50-2.45 (m, 2H), 2.40-2.30 (m, 2H), 1.53-1.43 (m, 2H), 1.20 (t, J=7.2 Hz, 3H), 1.02 (t, J=7.2 Hz, 3H), 0.84 (t, J=7.2 Hz, 3H).

### Example 6

### 2-(ethoxymethyl)-7-[4-[[ethyl(methyl)amino]methyl]phenyl]sulfanyl-6-methyl-1H-imidazo[4,5-c]pyridin-4-amine

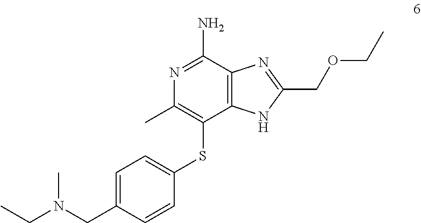 [SMILES: CCOCc1nc2c(N)nc(C)c(Sc3ccc(CN(C)CC)cc3)c2[nH]1]

### Step 1: Preparation of S-[4-[[ethyl(methyl)amino]methyl]phenyl]N,N-dimethylcarbamothioate (Compound 6a)

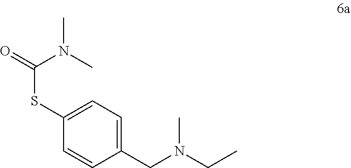 [SMILES: CCN(C)Cc1ccc(SC(=O)N(C)C)cc1]

A mixture of S-(4-formylphenyl) N,N-dimethylcarbamothioate (1.0 g, Compound 1i, 4.78 mmol), N-ethylmethylamine (0.37 g, 6.21 mmol) and acetic acid (0.09 g, 1.43 mmol) in DCM (10.0 mL) was stirred at 20° C. for 12 h. Then to the mixture was added NaBH(OAc)₃ (2.03 g, 9.56 mmol) at 20° C. The mixture was stirred at 20° C. for 2 h, then filtered through a pad of celite and washed with MeOH (20 mL). The filtrate was concentrated in vacuo. The residue was purified by prep-HPLC to afford S-[4-[[ethyl(methyl)amino]methyl]phenyl]N,N-dimethylcarbamothioate (1.0 g, Compound 6a) as a yellow oil. MS obsd. (ESI⁺) [(M+H)⁺]: 253.1.

### Step 2: Preparation of N-[[4-[[4-[[ethyl(methyl)amino]methyl]phenyl]disulfanyl]phenyl]methyl]-N-methyl-ethanamine (Compound 6b)

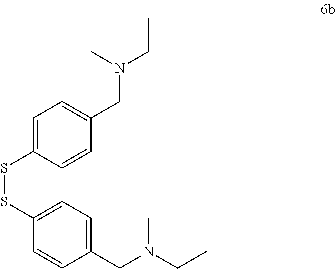 [SMILES: CCN(C)Cc1ccc(SSc2ccc(CN(C)CC)cc2)cc1]

Compound 6b was prepared in analogy to Example 1, Step 11 by using S-[4-[[ethyl(methyl)amino]methyl]phenyl]N,N-dimethylcarbamothioate (Compound 6a) instead of S-[4-[(dimethylamino)methyl]phenyl]N,N-dimethylcarbamothioate (Compound 1j). N-[[4-[[4-[[ethyl(methyl)amino]methyl]phenyl]disulfanyl]phenyl]methyl]-N-methyl-ethanamine (74 mg, Compound 6b) was obtained as a grey oil. MS obsd. (ESI⁺) [(M+H)⁺]: 361.3.

### Step 3: Preparation of 2-(ethoxymethyl)-7-[4-[[ethyl(methyl)amino]methyl]phenyl]sulfanyl-6-methyl-1H-imidazo[4,5-c]pyridin-4-amine (Example 6)

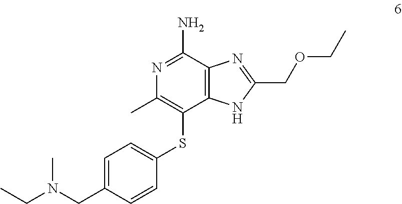 [SMILES: CCOCc1nc2c(N)nc(C)c(Sc3ccc(CN(C)CC)cc3)c2[nH]1]

To a mixture of 2-(ethoxymethyl)-7-iodo-6-methyl-1H-imidazo[4,5-c]pyridin-4-amine (35.0 mg, Compound 1g, 0.11 mmol), N-[[4-[[4-[[ethyl(methyl)amino]methyl]phenyl]disulfanyl]phenyl]methyl]-N-methyl-ethanamine (38.0 mg, Compound 6b, 0.11 mmol) and K₂CO₃ (14.57 mg, 0.11 mmol) in DMSO (2.0 mL) were added Cu₂S (16.77 mg, 0.11 mmol) and Fe powder (3.53 mg, 0.06 mmol) at 20° C. The suspension was degassed in vacuo and purged with N₂ three times. The mixture was stirred at 110° C. for 16 h under N₂ atmosphere. The reaction mixture was diluted with MeOH (5.0 mL), adjusted to pH=6 with FA at 0° C. and concentrated in vacuo. The residue was purified by prep-HPLC to afford 2-(ethoxymethyl)-7-[4-[[ethyl(methyl)amino]methyl]phenyl]sulfanyl-6-methyl-1H-imidazo[4,5-c]pyridin-4-amine (7.3 mg, Example 6) as a white solid. MS obsd. (ESI⁺) [(M+H)⁺]: 386.1. ¹H NMR (400 MHz, CD₃OD) δ ppm: 7.17 (d, J=8.4 Hz, 2H), 6.95 (d, J=8.4 Hz, 2H), 4.63 (s, 2H), 3.62-3.54 (m, 2H), 3.45 (s, 2H), 2.52 (s, 3H), 2.47-2.41 (m, 2H), 2.15 (s, 3H), 1.20 (t, J=7.2 Hz, 3H), 1.08 (t, J=7.2 Hz, 3H).

### Example 7

### 7-[4-[(dimethylamino)methyl]phenyl]sulfanyl-2-(ethoxymethyl)-1,6-dimethyl-imidazo[4,5-c]pyridin-4-amine

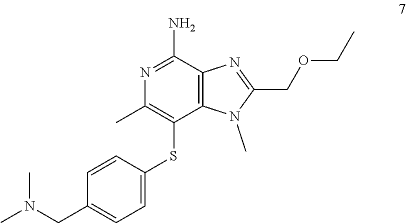 [SMILES: CCOCc1nc2c(N)nc(C)c(Sc3ccc(CN(C)C)cc3)c2n1C]

### Step 1: Preparation of N,N-bis[(2,4-dimethoxyphenyl)methyl]-2-(ethoxymethyl)-1,6-dimethyl-imidazo[4,5-c]pyridin-4-amine (Compound 7a)

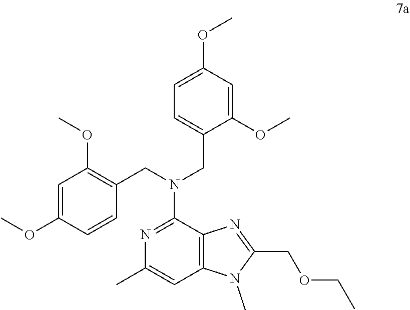 [SMILES: CCOCc1nc2c(N(Cc3ccc(OC)cc3OC)Cc3ccc(OC)cc3OC)nc(C)cc2n1C]

To a solution of N,N-bis[(2,4-dimethoxyphenyl)methyl]-2-(ethoxymethyl)-6-methyl-1H-imidazo[4,5-c]pyridin-4-amine (4.0 g, Compound 1e, 7.9 mmol) in DMF (40 mL) were added potassium carbonate (2.18 g, 15.79 mmol) and iodomethane (1.23 mL, 19.74 mmol) at 25° C. The mixture was stirred at 25° C. for 2 h, then poured into water (200 mL) and extracted with EA (3×150 mL). The organic layer was washed with brine (3×100 mL), dried over Na₂SO₄ and filtered. The filtrate was concentrated in vacuo to afford N,N-bis[(2,4-dimethoxyphenyl)methyl]-2-(ethoxymethyl)-1,6-dimethyl-imidazo[4,5-c]pyridin-4-amine (4.0 g, Compound 7a) as a yellow oil. MS obsd. (ESI⁺) [(M+H)⁺]: 521.4.

### Step 2: Preparation of 2-(ethoxymethyl)-1,6-dimethyl-imidazo[4,5-c]pyridin-4-amine (Compound 7b)

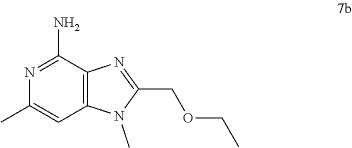 [SMILES: CCOCc1nc2c(N)nc(C)cc2n1C]

A solution of N,N-bis[(2,4-dimethoxyphenyl)methyl]-2-(ethoxymethyl)-1,6-dimethyl-imidazo[4,5-c]pyridin-4-amine (4.0 g, Compound 7a, 7.68 mmol) and TFA (40.0 mL) in DCM (40.0 mL) was stirred at 20° C. for 2 h. The reaction mixture was concentrated in vacuo. The pH of the residue was adjusted to 8 with sat. NaHCO₃. After lyophilisation, the residue was washed with DCM (160 mL) and MeOH (20 mL). The filtrate was concentrated in vacuo to afford 2-(ethoxymethyl)-1,6-dimethyl-imidazo[4,5-c]pyridin-4-amine (2.5 g, Compound 7b) as a light yellow solid, which was used for next step directly without further purification.

### Step 3: Preparation of 2-(ethoxymethyl)-7-iodo-1,6-dimethyl-imidazo[4,5-c]pyridin-4-amine (Compound 7c)

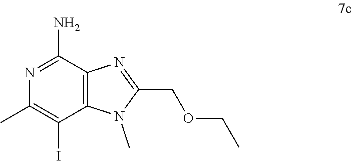 [SMILES: CCOCc1nc2c(N)nc(C)c(I)c2n1C]

To a solution of 2-(ethoxymethyl)-1,6-dimethyl-imidazo[4,5-c]pyridin-4-amine (2.45 g, Compound 7b, 7.33 mmol) in acetic acid (30 mL) was added NIS (3.3 g, 14.66 mmol) at 20° C. The reaction mixture was stirred at 20° C. for 2 h. The reaction mixture was concentrated in vacuo. The residue was purified by reversed prep-HPLC to afford 2-(ethoxymethyl)-7-iodo-1,6-dimethyl-imidazo[4,5-c]pyridin-4-amine (876.0 mg, Compound 7c) as a light yellow solid. MS obsd. (ESI⁺) [(M+H)⁺]: 346.9.

### Step 4: Preparation of 7-[4-[(dimethylamino)methyl]phenyl]sulfanyl-2-(ethoxymethyl)-1,6-dimethyl-imidazo[4,5-c]pyridin-4-amine (Example 7)

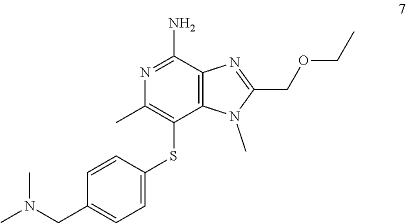 [SMILES: CCOCc1nc2c(N)nc(C)c(Sc3ccc(CN(C)C)cc3)c2n1C]

The title compound was prepared in analogy to Example 1, Step 12 by using 2-(ethoxymethyl)-7-iodo-1,6-dimethyl-imidazo[4,5-c]pyridin-4-amine (Compound 7c) instead of 2-(ethoxymethyl)-7-iodo-6-methyl-1H-imidazo[4,5-c]pyridin-4-amine (Compound 1g). 7-[4-[(Dimethylamino)methyl]phenyl]sulfanyl-2-(ethoxymethyl)-1,6-dimethyl-imidazo[4,5-c]pyridin-4-amine (31.6 mg, Example 7) was obtained as a white solid. MS obsd. (ESI⁺) [(M+H)⁺]: 386.2. ¹H NMR (400 MHz, CD₃OD) δ ppm: 7.41 (d, J=7.6 Hz, 2H), 7.12 (d, J=7.6 Hz, 2H), 4.71 (s, 2H), 4.21 (s, 2H), 3.99 (s, 3H), 3.63-3.55 (m, 2H), 2.79 (s, 6H), 2.60 (s, 3H), 1.19 (t, J=7.2 Hz, 3H).

### Example 8

### 2-[[4-amino-2-(ethoxymethyl)-6-methyl-1H-imidazo[4,5-c]pyridin-7-yl]sulfanyl]-5-[(dimethylamino)methyl]phenol

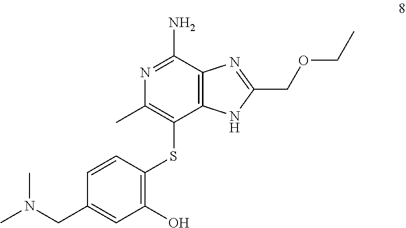 [SMILES: CCOCc1nc2c(N)nc(C)c(Sc3ccc(CN(C)C)cc3O)c2[nH]1]

### Step 1: Preparation of 5-iodo-6-methyl-3-nitro-pyridine-2,4-diol (Compound 8a)

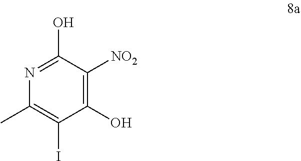

A mixture of 6-methyl-3-nitro-pyridine-2,4-diol (10.0 g, 58.78 mmol), HIO₄ (4.51 g, 23.51 mmol) in acetic acid (50 mL), H₂SO₄ (1.0 mL) and water (20 mL) was stirred at 90° C. for 0.5 h. Then to the mixture I₂ was added (5.96 g, 23.51 mmol) at 90° C. The mixture was stirred at 90° C. for 1 h. The mixture was cooled to room temperature, poured into sat. Na₂SO₃ (200 mL) and filtered. The collected solid was washed with H₂O (100 mL), dried in vacuo to afford 5-iodo-6-methyl-3-nitro-pyridine-2,4-diol (15.0 g, Compound 8a) as a yellow solid. MS obsd. (ESI⁺) [(M+H)⁺]: 296.9.

### Step 2: Preparation of 2,4-dichloro-5-iodo-6-methyl-3-nitro-pyridine (Compound 8b)

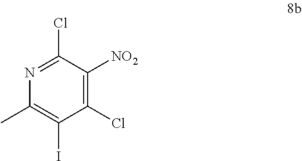

A mixture of 5-iodo-6-methyl-3-nitro-pyridine-2,4-diol (15.0 g, Compound 8a, 50.67 mmol) in phenyl dichlorophosphate (150 mL) was stirred at 180° C. for 1 h. The mixture was cooled to room temperature, poured into H₂O (600 mL), basified with solid NaHCO₃ to pH=7 and extracted with EA (3×300 mL). The organic layer was washed with brine (200 mL), dried over Na₂SO₄, filtered and concentrated in vacuo. The residue was purified by silica gel column to afford 2,4-dichloro-5-iodo-6-methyl-3-nitro-pyridine (15.0 g, Compound 8b) as a yellow solid. ¹H NMR (400 MHz, CDCl₃) δ ppm: 2.81 (s, 3H).

### Step 3: Preparation of 2-chloro-N-[(2,4-dimethoxyphenyl)methyl]-5-iodo-6-methyl-3-nitro-pyridin-4-amine (Compound 8c)

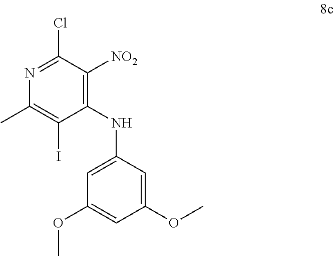

To a solution of 2,4-dichloro-5-iodo-6-methyl-3-nitro-pyridine (20.0 g, Compound 8b, 60.08 mmol) in THF (160 mL) were added DIEA (26.69 mL, 150.19 mmol) and 2,4-dimethoxybenzylamine (9.03 mL, 60.08 mmol) at 20° C. The mixture was stirred at 20° C. for 16 h. The mixture was diluted with H₂O (200 mL) and extracted with EA (3×300 mL). The organic layer was dried over Na₂SO₄, filtered and concentrated in vacuo. The residue was purified by silica gel column to afford 2-chloro-N-[(2,4-dimethoxyphenyl)methyl]-5-iodo-6-methyl-3-nitro-pyridin-4-amine (25.0 g, Compound 8c) as a yellow solid. MS obsd. (ESI⁺) [(M+H)⁺]: 464.0.

### Step 4: Preparation of N2,N2,N4-tris[(2,4-dimethoxyphenyl)methyl]-5-iodo-6-methyl-3-nitro-pyridine-2,4-diamine (Compound 8d)

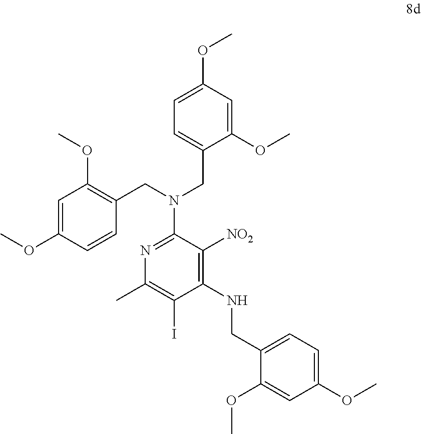

Compound 8d was prepared in analogy to Example 1, Step 2 by using 2-chloro-N-[(2,4-dimethoxyphenyl)methyl]-5-iodo-6-methyl-3-nitro-pyridin-4-amine (Compound 8c) instead of 2-chloro-6-methyl-3-nitro-pyridin-4-amine (Compound 1a). N2,N2,N4-tris[(2,4-dimethoxyphenyl)methyl]-5-iodo-6-methyl-3-nitro-pyridine-2,4-diamine (30.0 g, Compound 8d) was obtained as a yellow oil. MS obsd. (ESI⁺) [(M+H)⁺]: 744.9.

### Step 5: Preparation of N2,N2,N4-tris[(2,4-dimethoxyphenyl)methyl]-5-iodo-6-methyl-pyridine-2,3,4-triamine (Compound 8e)

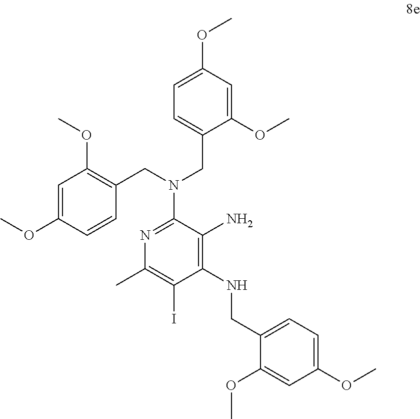

To a solution of N2,N2,N4-tris[(2,4-dimethoxyphenyl)methyl]-5-iodo-6-methyl-3-nitro-pyridine-2,4-diamine (25.0 g, Compound 8d, 33.58 mmol) in ethanol (150 mL), THF (200 mL) and water (150 mL) were added Fe (18.75 g, 335.76 mmol) and NH₄Cl (17.96 g, 335.76 mmol, 10.0 eq) at 20° C. The suspension was heated to 70° C. and stirred at 70° C. for 2.5 h. The mixture was filtered through a pad of celite and washed with MeOH (5×200 mL). The filtrate was concentrated in vacuo. The residue was washed with DCM/MeOH (300 mL, V/V=10/1) and filtered. The filtrate was concentrated in vacuo to afford N2,N2,N4-tris[(2,4-dimethoxyphenyl)methyl]-5-iodo-6-methyl-pyridine-2,3,4-triamine (20.0 g, Compound 8e) as a yellow oil. MS obsd. (ESI⁺) [(M+H)⁺]: 715.0.

### Step 6: Preparation of N-[2-[bis[(2,4-dimethoxyphenyl)methyl]amino]-4-[(2,4-dimethoxyphenyl)methylamino]-5-iodo-6-methyl-3-pyridyl]-2-ethoxy-acetamide (Compound 8f)

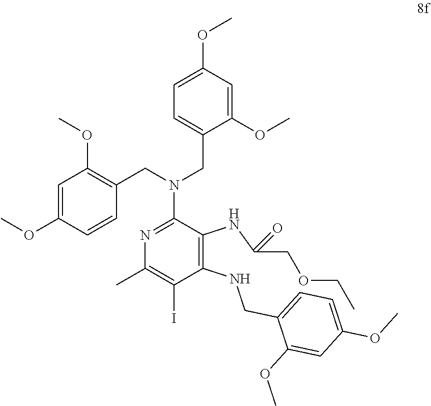

Compound 8f was prepared in analogy to Example 1, Step 4 by using N2,N2,N4-tris[(2,4-dimethoxyphenyl)methyl]-5-iodo-6-methyl-pyridine-2,3,4-triamine (Compound 8e) instead of N2,N2-bis[(2,4-dimethoxyphenyl)methyl]-6-methyl-pyridine-2,3,4-triamine (Compound 1c). N-[2-[bis[(2,4-dimethoxyphenyl)methyl]amino]-4-[(2,4-dimethoxyphenyl)methylamino]-5-iodo-6-methyl-3-pyridyl]-2-ethoxy-acetamide (10.0 g, Compound 8f) was obtained as a brown oil, which was used for next step directly without further purification.

### Step 7: Preparation of N,N,1-tris[(2,4-dimethoxyphenyl)methyl]-2-(ethoxymethyl)-7-iodo-6-methyl-imidazo[4,5-c]pyridin-4-amine (Compound 8g)

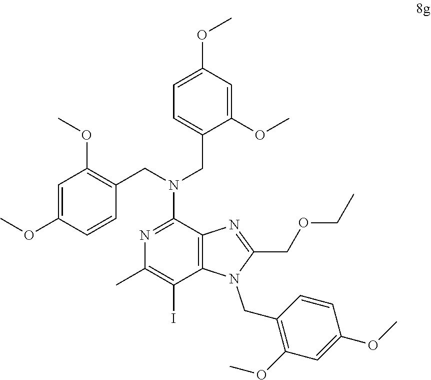

To a solution of N-[2-[bis[(2,4-dimethoxyphenyl)methyl]amino]-4-[(2,4-dimethoxyphenyl)methylamino]-5-iodo-6-methyl-3-pyridyl]-2-ethoxy-acetamide (10.0 g, Compound 8f, 12.49 mmol) in ethanol (100 mL) and water (100 mL) was added NaOH (5.07 g, 124.89 mmol) at 20° C. The mixture was stirred at 80° C. for 12 h. Then the mixture was concentrated in vacuo. The residue was purified by reversed prep-HPLC to afford N,N,1-tris[(2,4-dimethoxyphenyl)methyl]-2-(ethoxymethyl)-7-iodo-6-methyl-imidazo[4,5-c]pyridin-4-amine (5.00 g, Compound 8g) as a yellow solid.

### Step 8: Preparation of O-(4-formyl-2-methoxy-phenyl) N,N-dimethylcarbamothioate (Compound 8h)

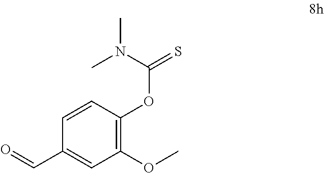 [SMILES: COc1cc(C=O)ccc1OC(=S)N(C)C]

Compound 8h was prepared in analogy to Example 1, Step 8 by using vanillin instead of 4-hydroxybenzaldehyde. O-(4-formyl-2-methoxy-phenyl) N,N-dimethylcarbamothioate (5.0 g, Compound 8h) was obtained as a light yellow solid. MS obsd. (ESI⁺) [(M+H)⁺]: 240.0. ¹H NMR (400 MHz, CDCl₃) δ ppm: 9.96 (s, 1H), 7.54-7.48 (m, 2H), 7.23 (d, J=8.4 Hz, 1H), 3.90 (s, 3H), 3.47 (s, 3H), 3.38 (s, 3H).

### Step 9: Preparation of S-(4-formyl-2-methoxyphenyl) N,N-dimethylcarbamothioate (Compound 8i)

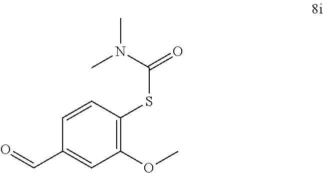 [SMILES: COc1cc(C=O)ccc1SC(=O)N(C)C]

Compound 8i was prepared in analogy to Example 1, Step 9 by using O-(4-formyl-2-methoxy-phenyl) N,N-dimethylcarbamothioate (Compound 8h) instead of O-(4-formylphenyl) N,N-dimethylcarbamothioate (Compound 1h). S-(4-formyl-2-methoxy-phenyl) N,N-dimethylcarbamothioate (4.0 g, Compound 8i) was obtained as a light yellow solid. ¹H NMR (400 MHz, CDCl₃) δ ppm: 10.00 (s, 1H), 7.69 (d, J=7.6 Hz, 1H), 7.49-7.43 (m, 2H), 3.95 (s, 3H), 3.14 (s, 3H), 3.03 (s, 3H).

### Step 10: Preparation of S-[4-[(dimethylamino)methyl]-2-methoxy-phenyl]N,N-dimethylcarbamothioate (Compound 8j)

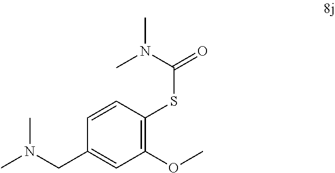 [SMILES: COc1cc(CN(C)C)ccc1SC(=O)N(C)C]

Compound 8j was prepared in analogy to Example 1, Step 10 by using S-(4-formyl-2-methoxy-phenyl) N,N-dimethylcarbamothioate (Compound 8i) instead of S-(4-formylphenyl) N,N-dimethylcarbamothioate (Compound 1i). S-[4-[(dimethylamino)methyl]-2-methoxy-phenyl]N,N-dimethylcarbamothioate (2.7 g, Compound 8j) was obtained as a light yellow oil. MS obsd. (ESI⁺) [(M+H)⁺]: 269.1.

### Step 11: Preparation of 4-[(dimethylamino)methyl]-2-methoxy-benzenethiol (Compound 8k)

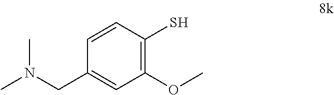

Compound 8k was prepared in analogy to Example 1, Step 11 by using S-[4-[(dimethylamino)methyl]-2-methoxy-phenyl]N,N-dimethylcarbamothioate (Compound 8j) instead of S-[4-[(dimethylamino)methyl]phenyl]N,N-dimethylcarbamothioate (Compound 1j). 4-[(Dimethylamino)methyl]-2-methoxy-benzenethiol (300.0 mg, Compound 8k) was obtained as a light yellow oil. MS obsd. (ESI⁺) [(M+H)⁺]: 198.0.

### Step 12: Preparation of 5-[(dimethylamino)methyl]-2-sulfanyl-phenol (Compound 81)

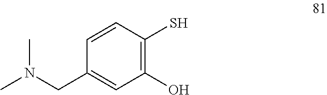

To a solution of 4-[(dimethylamino)methyl]-2-methoxy-benzenethiol (300.0 mg, Compound 8k, 1.52 mmol) in DCM (5.0 mL) was added BBr₃ (1.14 g, 4.56 mmol) dropwise at 0° C. The reaction mixture was stirred at 15° C. for 2 h. Then the reaction was quenched with H₂O (20 mL) at 5° C. and concentrated in vacuo. The residue was purified by prep-HPLC to afford 5-[(dimethylamino)methyl]-2-sulfanyl-phenol (270.0 mg, Compound 81) as a light yellow oil. MS obsd. (ESI⁺) [(M+H)⁺]: 184.1.

### Step 13: Preparation of 2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-5-[(dimethylamino)methyl]phenol (Compound 8m)

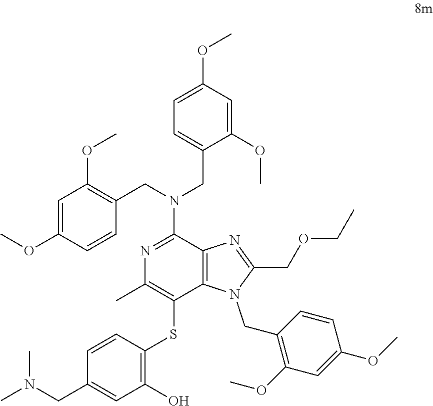

Compound 8m was prepared in analogy to Example 6, Step 3 by using N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g) and 5-[(dimethylamino)methyl]-2-sulfanyl-phenol (Compound 81) instead of N-[[4-[[4-[[ethyl(methyl)amino]methyl]phenyl]disulfanyl]phenyl]methyl]-N-methyl-ethanamine (Compound 6b). 2-[4-[Bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-5-[(dimethylamino)methyl]phenol (400.0 mg, Compound 8m) was obtained as a yellow solid. MS obsd. (ESI⁺) [(M+H)⁺]: 838.2.

### Step 14: Preparation of 2-[[4-amino-2-(ethoxymethyl)-6-methyl-1H-imidazo[4,5-c]pyridin-7-yl]sulfanyl]-5-[(dimethylamino)methyl]phenol (Example 8)

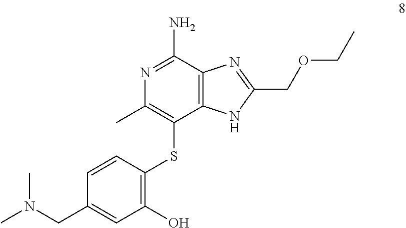 [SMILES: CCOCc1nc2c(N)nc(C)c(Sc3ccc(CN(C)C)cc3O)c2[nH]1]

A solution of 2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-5-[(dimethylamino)methyl]phenol (300.0 mg, Compound 8m, 0.36 mmol) and TFA (5.0 mL) in DCM (5.0 mL) was stirred at 20° C. for 2 h. The reaction mixture was concentrated in vacuo. The residue was purified by prep-HPLC to afford 2-[[4-amino-2-(ethoxymethyl)-6-methyl-1H-imidazo[4,5-c]pyridin-7-yl]sulfanyl]-5-[(dimethylamino)methyl]phenol (73.2 mg, Example 8) as a white solid. MS obsd. (ESI⁺) [(M+H)⁺]: 388.2. ¹H NMR (400 MHz, CD₃OD) δ ppm: 6.77 (d, J=1.6 Hz, 1H), 6.58 (dd, J=8.0 Hz, 1.6 Hz, 1H), 6.43 (d, J=8.0 Hz, 1H), 4.63 (s, 2H), 3.58 (q, J=6.8 Hz, 2H), 3.34 (s, 2H), 2.53 (s, 3H), 2.20 (s, 6H), 1.21 (t, J=6.8 Hz, 3H).

### Example 9

### 7-[4-[(dimethylamino)methyl]-3-methoxy-phenyl]sulfanyl-2-(ethoxymethyl)-6-methyl-1H-imidazo[4,5-c]pyridin-4-amine

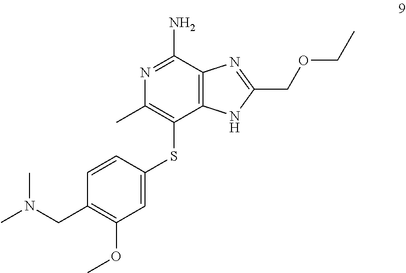 [SMILES: CCOCc1nc2c(N)nc(C)c(Sc3ccc(CN(C)C)c(OC)c3)c2[nH]1]

### Step 1: Preparation of O-(4-formyl-3-methoxy-phenyl) N,N-dimethylcarbamothioate (Compound 9a)

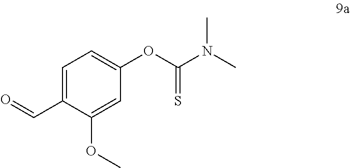 [SMILES: COc1cc(OC(=S)N(C)C)ccc1C=O]

Compound 9a was prepared in analogy to Example 1, Step 8 by using 4-hydroxy-2-methoxy-benzaldehyde instead of 4-hydroxybenzaldehyde. O-(4-formyl-3-methoxy-phenyl) N,N-dimethylcarbamothioate (11.0 g, Compound 9a) was obtained as a yellow solid. MS obsd. (ESI⁺) [(M+H)⁺]: 240.1. ¹H NMR (400 MHz, CDCl₃) δ ppm: 10.41 (s, 1H), 7.87 (d, J=8.4 Hz, 1H), 6.78-6.72 (m, 2H), 3.93 (s, 3H), 3.47 (s, 3H), 3.36 (s, 3H).

### Step 2: Preparation of S-(4-formyl-3-methoxy-phenyl) N,N-dimethylcarbamothioate (Compound 9b)

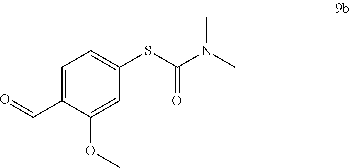 [SMILES: COc1cc(SC(=O)N(C)C)ccc1C=O]

A solution of O-(4-formyl-3-methoxy-phenyl) N,N-dimethylcarbamothioate (10.0 g, Compound 9a, 41.79 mmol) in Ph₂O (50.0 mL) was stirred at 190° C. for 6 h. The mixture was purified by silica gel column to afford S-(4-formyl-3-methoxy-phenyl) N,N-dimethylcarbamothioate (6.0 g, Compound 9b) as a yellow solid. MS obsd. (ESI⁺) [(M+H)⁺]: 240.1.

### Step 3: Preparation of 2-methoxy-4-sulfanyl-benzaldehyde (Compound 9c)

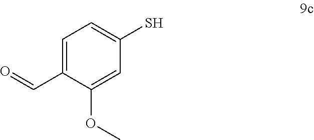

Compound 9c was prepared in analogy to Example 1, Step 11 by using S-(4-formyl-3-methoxy-phenyl) N,N-dimethylcarbamothioate (Compound 9b) instead of S-[4-[(dimethylamino)methyl]phenyl]N,N-dimethylcarbamothioate (Compound 1j). 2-Methoxy-4-sulfanyl-benzaldehyde (700.0 mg, Compound 9c) was obtained as a yellow solid. MS obsd. (ESI⁺) [(M+H)⁺]: 169.2. ¹H NMR (400 MHz, CDCl₃) δ ppm: 10.21 (s, 1H), 7.54 (d, J=8.0 Hz, 1H), 7.18 (s, 1H), 6.98 (d, J=8.0 Hz, 1H), 6.12 (br s, 1H), 3.89 (s, 3H).

### Step 4: Preparation of 4-[[4-amino-2-(ethoxymethyl)-6-methyl-1H-imidazo[4,5-c]pyridin-7-yl]sulfanyl]-2-methoxy-benzaldehyde (Compound 9d)

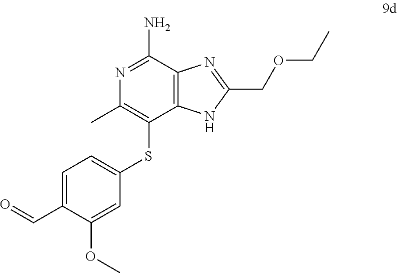 [SMILES: CCOCc1nc2c(N)nc(C)c(Sc3ccc(C=O)c(OC)c3)c2[nH]1]

Compound 9d was prepared in analogy to Example 5, Step 3 by using 2-methoxy-4-sulfanyl-benzaldehyde (Compound 9c) instead of N-ethyl-N-[[4-[[4-[[ethyl(propyl)amino]methyl]phenyl]disulfanyl]phenyl]methyl]propan-1-amine (Compound 5b). 4-[[4-Amino-2-(ethoxymethyl)-6-methyl-1H-imidazo[4,5-c]pyridin-7-yl]sulfanyl]-2-methoxy-benzaldehyde (150.0 mg, Compound 9d) was obtained as a yellow solid. MS obsd. (ESI⁺) [(M+H)⁺]: 373.3.

### Step 5: Preparation of 7-[4-[(dimethylamino)methyl]-3-methoxy-phenyl]sulfanyl-2-(ethoxymethyl)-6-methyl-1H-imidazo[4,5-c]pyridin-4-amine (Example 9)

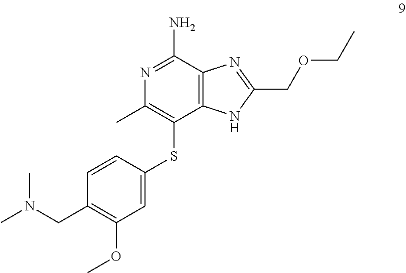 [SMILES: CCOCc1nc2c(N)nc(C)c(Sc3ccc(CN(C)C)c(OC)c3)c2[nH]1]

To a mixture of 4-[[4-amino-2-(ethoxymethyl)-6-methyl-1H-imidazo[4,5-c]pyridin-7-yl]sulfanyl]-2-methoxy-benzaldehyde (150.0 mg, Compound 9d, 0.4 mmol) and NaOAc (330.38 mg, 4.03 mmol) in methanol (5.0 mL) was added dimethylamine hydrochloride (328.4 mg, 4.03 mmol) at 20° C. The mixture was stirred at 20° C. for 12 h under N₂, then NaBH(OAc)₃ (341.43 mg, 1.61 mmol) was added at 20° C. The mixture was stirred at 20° C. for another 4 h, then filtered through celite and the filtrate was purified by prep-HPLC to afford 7-[4-[(dimethylamino)methyl]-3-methoxy-phenyl]sulfanyl-2-(ethoxymethyl)-6-methyl-1H-imidazo[4,5-c]pyridin-4-amine (30.0 mg, Example 9) as a yellow solid. MS obsd. (ESI⁺) [(M+H)⁺]: 402.1. ¹H NMR (400 MHz, DMSO-d₆) δ ppm: 12.58 (s, 1H), 7.10 (d, J=7.6 Hz, 1H), 6.73 (d, J=1.2 Hz, 1H), 6.57 (br s, 2H), 6.34 (dd, J=7.6 Hz, 1.2 Hz, 1H), 4.52 (s, 2H), 3.66 (s, 3H), 3.52-3.47 (m, 2H), 3.38 (s, 2H), 2.44 (s, 3H), 2.16 (s, 6H), 1.11 (t, J=6.8 Hz, 3H).

### Example 10

### 7-[2-[4-[(dimethylamino)methyl]phenyl]ethylsulfanyl]-2-(ethoxymethyl)-6-methyl-1H-imidazo[4,5-c]pyridin-4-amine

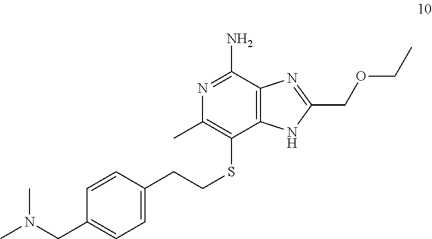 [SMILES: CCOCc1nc2c(N)nc(C)c(SCCc3ccc(CN(C)C)cc3)c2[nH]1]

### Step 1: Preparation of [4-(2-bromoethyl)phenyl]methanol (Compound 10a)

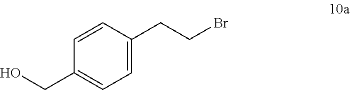 [SMILES: OCc1ccc(CCBr)cc1]

To a solution of 4-(2-bromoethyl)benzoic acid methyl ester (1.5 g, 6.17 mmol) in THF (30 mL) was added diisobutylaluminium hydride (12.96 mL, 12.96 mmol, 1.0 M) dropwise at −78° C. The mixture was stirred at 15° C. for 2 h. Then the reaction was quenched with saturated aq. NH₄Cl (100 mL) at 0° C. and extracted with EA (3×100 mL). The combined organic layer was washed with brine (100 mL), dried with Na₂SO₄ and filtered. The filtrate was concentrated in vacuo to afford [4-(2-bromoethyl)phenyl]methanol (1.2 g, Compound 10a) as a light yellow oil. ¹H NMR (400 MHz, CDCl₃) δ ppm: 7.34 (d, J=8.0 Hz, 2H), 7.22 (d, J=8.0 Hz, 2H), 4.68 (s, 2H), 3.58 (t, J=7.6 Hz, 2H), 3.18 (t, J=7.6 Hz, 2H).

### Step 2: Preparation of 4-(2-bromoethyl)benzaldehyde (Compound 10b)

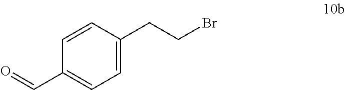 [SMILES: O=Cc1ccc(CCBr)cc1]

To a solution of [4-(2-bromoethyl)phenyl]methanol (1.0 g, Compound 10a, 4.65 mmol) in DCM (30.0 mL) was added Dess-Martin Periodinane (2.95 g, 6.97 mmol) at 20° C. under N₂. The reaction mixture was stirred at 20° C. for 4 h, then filtered and the filtrate was concentrated in vacuo. The residue was purified by silica gel column to afford 4-(2-bromoethyl)benzaldehyde (700.0 mg, Compound 10b) as a white solid. ¹H NMR (400 MHz, CDCl₃) δ ppm: 10.01 (s, 1H), 7.86 (d, J=8.0 Hz, 2H), 7.40 (d, J=8.0 Hz, 2H), 3.62 (t, J=7.6 Hz, 2H), 3.27 (t, J=7.6 Hz, 2H).

### Step 3: Preparation of 4-[2-[[4-amino-2-(ethoxymethyl)-6-methyl-1H-imidazo[4,5-c]pyridin-7-yl]sulfanyl]ethyl]benzaldehyde (Compound 10c)

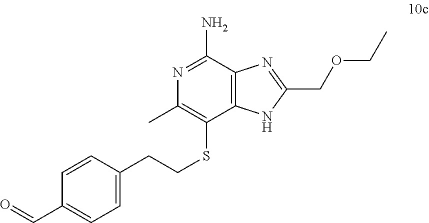 [SMILES: CCOCc1nc2c(N)nc(C)c(SCCc3ccc(C=O)cc3)c2[nH]1]

To a mixture of 2-(ethoxymethyl)-7-iodo-6-methyl-1H-imidazo[4,5-c]pyridin-4-amine (130.0 mg, Compound 1g, 0.39 mmol), Cs₂CO₃ (255.36 mg, 0.78 mmol) in 1,4-Dioxane (3.0 mL) were added triisopropylsilanethiol (96.89 mg, 0.51 mmol) and Pd XantPhos G3 (40.44 mg, 0.04 mmol) at 20° C. The mixture was degassed for three times under N₂ and stirred at 100° C. for 4 h under N₂ balloon. The mixture was cooled to room temperature. Then to the mixture was added 4-(2-bromoethyl)benzaldehyde (83.4 mg, Compound 10b, 0.39 mmol) in DMF (1.0 mL) at 20° C. under N₂. The mixture was stirred at 20° C. for 2 h, then filtered and the filtrate was concentrated in vacuo. The residue was purified by silica gel column to afford 4-[2-[[4-amino-2-(ethoxymethyl)-6-methyl-1H-imidazo[4,5-c]pyridin-7-yl]sulfanyl]ethyl]benzaldehyde (50.0 mg, Compound 10c) as a light yellow oil. MS obsd. (ESI⁺) [(M+H)⁺]: 371.1.

### Step 4: Preparation of 7-[2-[4-[(dimethylamino)methyl]phenyl]ethylsulfanyl]-2-(ethoxymethyl)-6-methyl-1H-imidazo[4,5-c]pyridin-4-amine (Example 10)

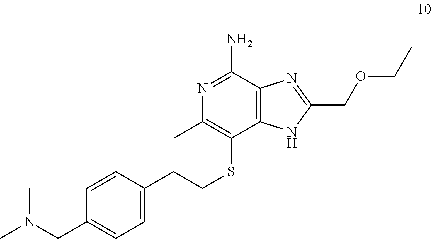 [SMILES: CCOCc1nc2c(N)nc(C)c(SCCc3ccc(CN(C)C)cc3)c2[nH]1]

A mixture of 4-[2-[[4-amino-2-(ethoxymethyl)-6-methyl-1H-imidazo[4,5-c]pyridin-7-yl]sulfanyl]ethyl]benzaldehyde (45.0 mg, Compound 10c, 0.12 mmol) and dimethylamine hydrochloride (12.88 mg, 0.16 mmol) in DCM (2.0 mL) was stirred at 20° C. for 11 h. Then to the mixture was added NaBH(OAc)₃ (51.49 mg, 0.24 mmol) at 20° C. The mixture was stirred at 20° C. for 1 h. Then the mixture was concentrated in vacuo. The residue was purified by prep-HPLC to afford 7-[2-[4-[(dimethylamino)methyl]phenyl]ethylsulfanyl]-2-(ethoxymethyl)-6-methyl-1H-imidazo[4,5-c]pyridin-4-amine (23.3 mg, Example 10) as a white solid. MS obsd. (ESI⁺) [(M+H)⁺]: 400.2. ¹H NMR (400 MHz, CD₃OD) δ ppm: 7.30 (d, J=8.0 Hz, 2H), 7.20 (d, J=8.0 Hz, 2H), 4.71 (s, 2H), 4.15 (s, 2H), 3.70-3.61 (m, 2H), 3.08 (t, J=7.2 Hz, 2H), 2.91 (t, J=7.2 Hz, 2H), 2.74 (s, 6H), 2.59 (s, 3H), 1.26 (t, J=7.2 Hz, 3H).

### Example 11

### 5-[[4-amino-2-(ethoxymethyl)-6-methyl-1H-imidazo[4,5-c]pyridin-7-yl]sulfanyl]-2-[(dimethylamino)methyl]phenol

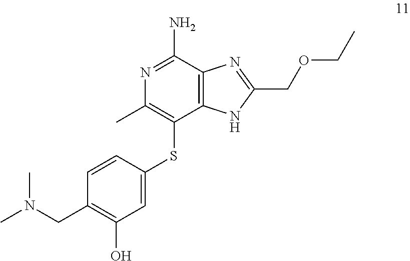 [SMILES: CCOCc1nc2c(N)nc(C)c(Sc3ccc(CN(C)C)c(O)c3)c2[nH]1]

### Step 1: Preparation of S-[4-[(dimethylamino)methyl]-3-methoxy-phenyl]N,N-dimethylcarbamothioate (Compound 11a)

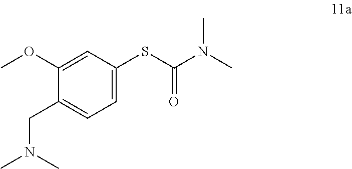

To a solution of S-(4-formyl-3-methoxy-phenyl) N,N-dimethylcarbamothioate (2.2 g, Compound 9b, 9.19 mmol) in DCM (20.0 mL) was added Me₂NH—HCl (2.99 g, 11.95 mmol) at 0° C. The reaction mixture was stirred at 20° C. for 2 h. Then to the mixture was added NaBH(OAc)₃ (3.89 g, 18.4 mmol) at 20° C. The mixture was stirred at 20° C. for 2 h. The reaction mixture was concentrated in vacuo. The residue was diluted with DCM (100 mL), washed with brine (60 mL), dried over Na₂SO₄, filtered and concentrated in vacuo. The residue was purified by reversed prep-HPLC to afford S-[4-[(dimethylamino)methyl]-3-methoxy-phenyl]N,N-dimethylcarbamothioate (1.7 g, Compound 11a) as alight yellow solid.

### Step 2: Preparation of 1-[4-[[4-[(dimethylamino)methyl]-3-methoxy-phenyl]disulfanyl]-2-methoxy-phenyl]-N,N-dimethyl-methanamine (Compound 11b)

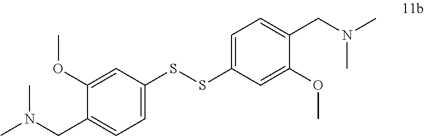

Compound 11b was prepared in analogy to Example 1, Step 11 by using S-[4-[(dimethylamino)methyl]-3-methoxy-phenyl]N,N-dimethylcarbamothioate (Compound 11a) instead of S-[4-[(dimethylamino)methyl]phenyl]N,N-dimethylcarbamothioate (Compound 1j). 1-[4-[[4-[(Dimethylamino)methyl]-3-methoxy-phenyl]disulfanyl]-2-methoxy-phenyl]-N,N-dimethyl-methanamine (1.5 g, Compound 11b) was obtained as a colorless oil. MS obsd. (ESI⁺) [(M+H)⁺]: 393.2.

### Step 3: Preparation of 2-[(dimethylamino)methyl]-5-[[4-[(dimethylamino)methyl]-3-hydroxy-phenyl]disulfanyl]phenol (Compound 11c)

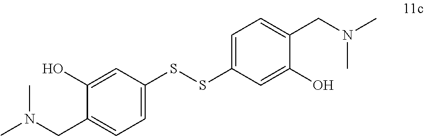

To a solution of 1-[4-[[4-[(dimethylamino)methyl]-3-methoxy-phenyl]disulfanyl]-2-methoxy-phenyl]-N,N-dimethyl-methanamine (500.0 mg, Compound 11b, 1.27 mmol) in DCM (5.0 mL) was added BBr₃ (957.21 mg, 3.82 mmol) dropwise at 0° C. The reaction mixture was stirred at 20° C. for 12 h. Then the reaction was quenched with MeOH (20 mL) dropwise at 0° C.

The mixture was concentrated in vacuo. The residue was purified by reversed prep-HPLC to afford 2-[(dimethylamino)methyl]-5-[[4-[(dimethylamino)methyl]-3-hydroxy-phenyl]disulfanyl]phenol (460.0 mg, Compound 11c) as a brown oil. MS obsd. (ESI⁺) [(M+H)⁺]: 365.2.

### Step 4: Preparation of 5-[[4-amino-2-(ethoxymethyl)-6-methyl-1H-imidazo[4,5-c]pyridin-7-yl]sulfanyl]-2-[(dimethylamino)methyl]phenol (Example 11)

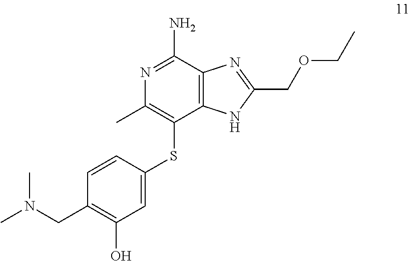 [SMILES: CCOCc1nc2c(N)nc(C)c(Sc3ccc(CN(C)C)c(O)c3)c2[nH]1]

The title compound was prepared in analogy to Example 5, Step 3 by using 2-[(dimethylamino)methyl]-5-[[4-[(dimethylamino)methyl]-3-hydroxy-phenyl]disulfanyl]phenol (Compound 11c) instead of N-ethyl-N-[[4-[[4-[[ethyl(propyl)amino]methyl]phenyl]disulfanyl]phenyl]methyl]propan-1-amine (Compound 5b). 5-[[4-Amino-2-(ethoxymethyl)-6-methyl-1H-imidazo[4,5-c]pyridin-7-yl]sulfanyl]-2-[(dimethylamino)methyl]phenol (10.5 mg, Example 11) was obtained as a white solid. MS obsd. (ESI⁺) [(M+H)⁺]: 388.1. ¹H NMR (400 MHz, CD₃OD) δ ppm: 6.91 (d, J=8.0 Hz, 1H), 6.45 (dd, J=8.0 Hz, 2.0 Hz, 1H), 6.32 (d, J=2.0 Hz, 1H), 4.63 (s, 2H), 3.61-3.55 (m, 4H), 2.52 (s, 3H), 2.29 (s, 6H), 1.21 (t, J=7.2 Hz, 3H).

### Example 12

### 5-[4-amino-2-(ethoxymethyl)-1,6-dimethyl-imidazo[4,5-c]pyridin-7-yl]sulfanyl-2-[(dimethylamino)methyl]phenol

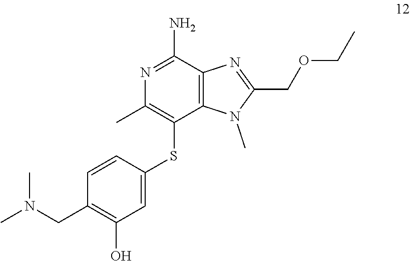 [SMILES: CCOCc1nc2c(N)nc(C)c(Sc3ccc(CN(C)C)c(O)c3)c2n1C]

### Step 1: Preparation of 2-chloro-5-iodo-6-methyl-3-nitro-pyridin-4-amine (Compound 12a)

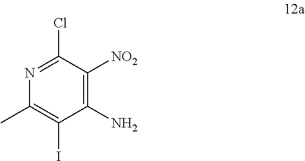

A solution of 2,4-dichloro-5-iodo-6-methyl-3-nitro-pyridine (15.0 g, Compound 8b, 45.06 mmol) in NH₃/MeOH (50.0 mL, 350.0 mmol, 7.0 M) was stirred at 25° C. for 12 h. The mixture was concentrated in vacuo. The residue was purified by silica gel column to afford 2-chloro-5-iodo-6-methyl-3-nitro-pyridin-4-amine (7.00 g, Compound 12a) as a yellow solid. ¹H NMR (400 MHz, DMSO-d₆) δ ppm: 7.08 (s, 2H), 2.57 (s, 3H).

### Step 2: Preparation of N2,N2-bis[(2,4-dimethoxyphenyl)methyl]-5-iodo-6-methyl-3-nitro-pyridine-2,4-diamine (Compound 12b)

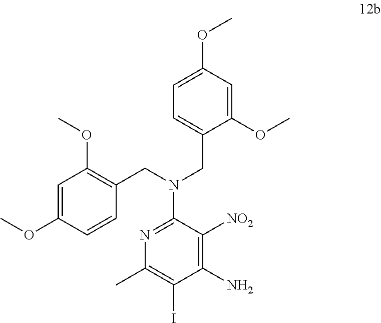

Compound 12b was prepared in analogy to Example 1, Step 2 by using 2-chloro-5-iodo-6-methyl-3-nitro-pyridin-4-amine (Compound 12a) instead of 2-chloro-6-methyl-3-nitro-pyridin-4-amine (Compound 1a). N2,N2-bis[(2,4-dimethoxyphenyl)methyl]-5-iodo-6-methyl-3-nitro-pyridine-2,4-diamine (10.0 g, Compound 12b) was obtained as a light red oil. ¹H NMR (400 MHz, CDCl₃) δ ppm: 7.09 (d, J=8.0 Hz, 2H), 6.67 (br s, 2H), 6.44-6.34 (m, 4H), 4.39 (s, 4H), 3.79 (s, 6H), 3.65 (s, 6H), 2.55 (s, 3H).

### Step 3: Preparation of N2,N2-bis[(2,4-dimethoxyphenyl)methyl]-5-iodo-6-methyl-pyridine-2,3,4-triamine (Compound 12c)

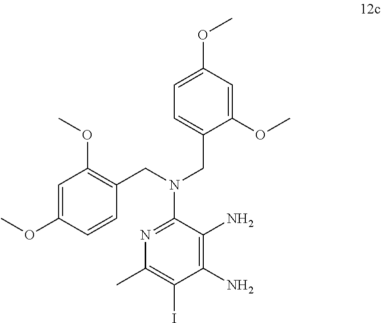

Compound 12c was prepared in analogy to Example 8, Step 5 by using N2,N2-bis[(2,4-dimethoxyphenyl)methyl]-5-iodo-6-methyl-3-nitro-pyridine-2,4-diamine (Compound 12b) instead of N2,N2,N4-tris[(2,4-dimethoxyphenyl)methyl]-5-iodo-6-methyl-3-nitro-pyridine-2,4-diamine (Compound 8d). N2,N2-bis[(2,4-dimethoxyphenyl)methyl]-5-iodo-6-methyl-pyridine-2,3,4-triamine (8.4 g, Compound 12c) was obtained as a light brown solid, which was used for next step directly without further purification.

### Step 4: Preparation of N-[4-amino-2-[bis[(2,4-dimethoxyphenyl)methyl]amino]-5-iodo-6-methyl-3-pyridyl]-2-ethoxy-acetamide (Compound 12d)

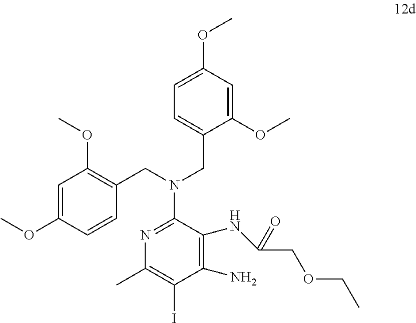 [SMILES: CCOCC(=O)Nc1c(N(Cc2ccc(OC)cc2OC)Cc2ccc(OC)cc2OC)nc(C)c(I)c1N]

Compound 12d was prepared in analogy to Example 1, Step 4 by using N2,N2-bis[(2,4-dimethoxyphenyl)methyl]-5-iodo-6-methyl-pyridine-2,3,4-triamine (Compound 12c) instead of N2,N2-bis[(2,4-dimethoxyphenyl)methyl]-6-methyl-pyridine-2,3,4-triamine (Compound 1c). N-[4-amino-2-[bis[(2,4-dimethoxyphenyl)methyl]amino]-5-iodo-6-methyl-3-pyridyl]-2-ethoxy-acetamide (9.20 g, Compound 12d) was obtained as a light yellow oil, which was used for next step directly without further purification.

### Step 5: Preparation of N,N-bis[(2,4-dimethoxyphenyl)methyl]-2-(ethoxymethyl)-7-iodo-6-methyl-1H-imidazo[4,5-c]pyridin-4-amine (Compound 12e)

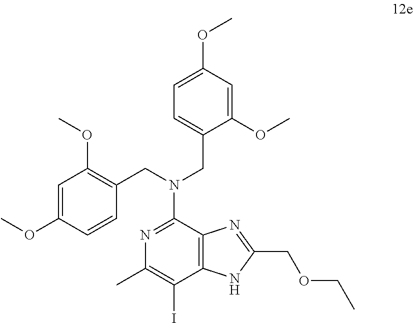 [SMILES: CCOCc1nc2c(N(Cc3ccc(OC)cc3OC)Cc3ccc(OC)cc3OC)nc(C)c(I)c2[nH]1]

Compound 12e was prepared in analogy to Example 1, Step 5 by using N-[4-amino-2-[bis[(2,4-dimethoxyphenyl)methyl]amino]-5-iodo-6-methyl-3-pyridyl]-2-ethoxy-acetamide (Compound 12d) instead of N-[4-amino-2-[bis[(2,4-dimethoxyphenyl)methyl]amino]-6-methyl-3-pyridyl]-2-ethoxy-acetamide (Compound 1d). N,N-bis[(2,4-dimethoxyphenyl)methyl]-2-(ethoxymethyl)-7-iodo-6-methyl-1H-imidazo[4,5-c]pyridin-4-amine (5.00 g, Compound 12e) was obtained as a light yellow solid. MS obsd. (ESI⁺) [(M+H)⁺]: 633.3.

### Step 6: Preparation of N,N-bis[(2,4-dimethoxyphenyl)methyl]-2-(ethoxymethyl)-7-iodo-1,6-dimethyl-imidazo[4,5-c]pyridin-4-amine (Compound 12f)

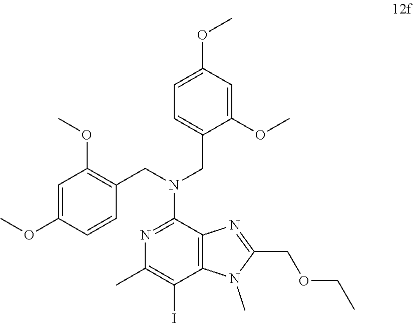 [SMILES: CCOCc1nc2c(N(Cc3ccc(OC)cc3OC)Cc3ccc(OC)cc3OC)nc(C)c(I)c2n1C]

Compound 12f was prepared in analogy to Example 7, Step 1 by using N,N-bis[(2,4-dimethoxyphenyl)methyl]-2-(ethoxymethyl)-7-iodo-6-methyl-1H-imidazo[4,5-c]pyridin-4-amine (Compound 12e) instead of N,N-bis[(2,4-dimethoxyphenyl)methyl]-2-(ethoxymethyl)-6-methyl-1H-imidazo[4,5-c]pyridin-4-amine (Compound 1e). N,N-bis[(2,4-dimethoxyphenyl)methyl]-2-(ethoxymethyl)-7-iodo-1,6-dimethyl-imidazo[4,5-c]pyridin-4-amine (600.0 mg, Compound 12f) was obtained as a light yellow solid. MS obsd. (ESI⁺) [(M+H)⁺]: 647.2.

### Step 7: Preparation of 5-[4-[bis[(2,4-dimethoxyphenyl)methyl]amino]-2-(ethoxymethyl)-1,6-dimethyl-imidazo[4,5-c]pyridin-7-yl]sulfanyl-2-[(dimethylamino)methyl]phenol (Compound 12g)

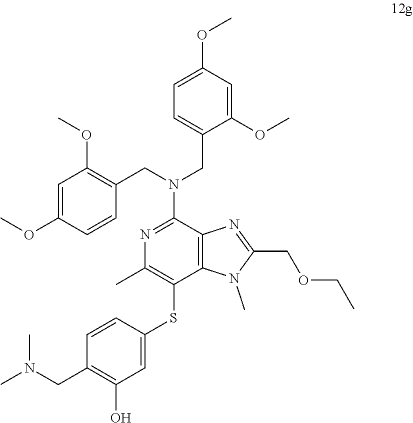 [SMILES: CCOCc1nc2c(N(Cc3ccc(OC)cc3OC)Cc3ccc(OC)cc3OC)nc(C)c(Sc3ccc(CN(C)C)c(O)c3)c2n1C]

To a mixture of 2-[(dimethylamino)methyl]-5-[[4-[(dimethylamino)methyl]-3-hydroxy-phenyl]disulfanyl]phenol (51.31 mg, Compound 11c, 0.14 mmol), N,N-bis[(2,4-dimethoxyphenyl)methyl]-2-(ethoxymethyl)-7-iodo-1,6-dimethyl-imidazo[4,5-c]pyridin-4-amine (130.0 mg, Compound 12f, 0.2 mmol) and K₂CO₃ (55.58 mg, 0.4 mmol) in DMSO (3.0 mL) were added Fe (11.41 mg, 0.2 mmol) and Cu₂S (19.21 mg, 0.12 mmol) at 20° C. The suspension was degassed in vacuo and purged with N₂ three times. The mixture was stirred at 100° C. for 16 h under N₂ atmosphere. The reaction mixture was purified by reversed prep-HPLC to afford 5-[4-[bis[(2,4-dimethoxyphenyl)methyl]amino]-2-(ethoxymethyl)-1,6-dimethyl-imidazo[4,5-c]pyridin-7-yl]sulfanyl-2-[(dimethylamino)methyl]phenol (38.0 mg, Compound 12g) as a light red oil. MS obsd. (ESI⁺) [(M+H)⁺]: 702.3.

### Step 8: Preparation of 5-[4-amino-2-(ethoxymethyl)-1,6-dimethyl-imidazo[4,5-c]pyridin-7-yl]sulfanyl-2-[(dimethylamino)methyl]phenol (Example 12)

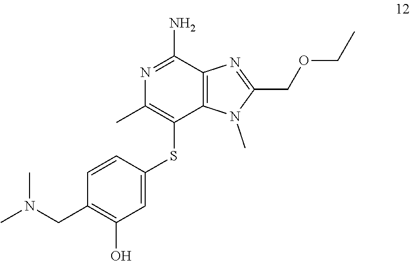 [SMILES: CCOCc1nc2c(N)nc(C)c(Sc3ccc(CN(C)C)c(O)c3)c2n1C]

A solution of 5-[4-[bis[(2,4-dimethoxyphenyl)methyl]amino]-2-(ethoxymethyl)-1,6-dimethyl-imidazo[4,5-c]pyridin-7-yl]sulfanyl-2-[(dimethylamino)methyl]phenol (38.0 mg, Compound 12g, 0.05 mmol) and TFA (2.0 mL) in DCM (2.0 mL) was stirred at 20° C. for 2 h. The reaction mixture was concentrated in vacuo. The residue was purified by prep-HPLC to afford 5-[4-amino-2-(ethoxymethyl)-1,6-dimethyl-imidazo[4,5-c]pyridin-7-yl]sulfanyl-2-[(dimethylamino)methyl]phenol (5.7 mg, Example 12) as a white solid. MS obsd. (ESI⁺) [(M+H)⁺]: 402.1. ¹H NMR (400 MHz, CD₃OD) δ ppm: 6.94 (d, J=8.0 Hz, 1H), 6.43 (dd, J=8.0 Hz, 2.0 Hz, 1H), 6.29 (d, J=2.0 Hz, 1H), 4.69 (s, 2H), 4.00 (s, 3H), 3.61-3.53 (m, 4H), 2.56 (s, 3H), 2.29 (s, 6H), 1.19 (t, J=7.2 Hz, 3H).

### Example 13

### 5-[2-[[4-amino-2-(ethoxymethyl)-6-methyl-LH-imidazo[4,5-c]pyridin-7-yl]sulfanyl]ethyl]-2-[(dimethylamino)methyl]phenol

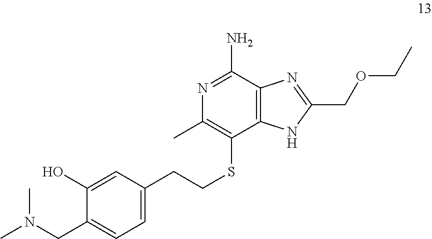 [SMILES: CCOCc1nc2c(N)nc(C)c(SCCc3ccc(CN(C)C)c(O)c3)c2[nH]1]

### Step 1: Preparation of methyl 2-(4-formyl-3-methoxy-phenyl)acetate (Compound 13a)

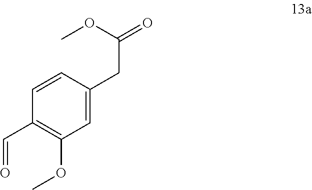

To a solution of 4-bromo-2-methoxybenzaldehyde (15.28 g, 71.06 mmol) in m-xylene (250.0 mL, 71.06 mmol) were added potassium 3-methoxy-3-oxopropanoate (16.65 g, 106.58 mmol), Pd₂(allyl)₂Cl₂ (519.98 mg, 1.42 mmol), BINAP (2.65 g, 4.26 mmol) and DMAP (868.1 mg, 7.11 mmol) at 25° C. The mixture was degassed in vacuo with N₂ three times. The suspension was stirred at 140° C. for 20 h under N₂. The suspension was concentrated in vacuo. The residue was purified by silica gel column to afford methyl 2-(4-formyl-3-methoxy-phenyl)acetate (2.09 g, Compound 13a) as a yellow oil. ¹H NMR (400 MHz, CDCl₃) δ ppm: 10.44 (s, 1H), 7.79 (d, J=8.0 Hz, 1H), 6.96 (d, J=8.0 Hz, 1H), 6.94 (s, 1H), 3.94 (s, 3H), 3.73 (s, 3H), 3.68 (s, 2H).

### Step 2: Preparation of methyl 2-[4-[(dimethylamino)methyl]-3-methoxy-phenyl]acetate (Compound 13b)

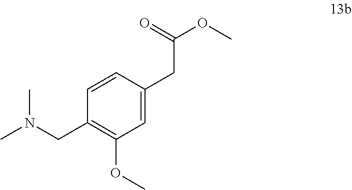

To a solution of methyl 2-(4-formyl-3-methoxy-phenyl)acetate (1.50 g, Compound 13a, 7.2 mmol) in DCE (50 mL) were added dimethylamine (36.02 mL, 72.04 mmol, 2.0 M in MeOH) and acetic acid (865.23 mg, 14.41 mmol) at 20° C. The mixture was stirred at 20° C. for 1 h. Then to the mixture was added NaBH(OAc)₃ (9.16 g, 43.23 mmol) at 20° C. The mixture was stirred at 20° C. for 2 h. The mixture was concentrated in vacuo and the residue was purified by reversed prep-HPLC to afford methyl 2-[4-[(dimethylamino)methyl]-3-methoxy-phenyl]acetate (907.0 mg, Compound 13b) as a yellow oil. ¹H NMR (400 MHz, CDCl₃) δ ppm: 7.22 (d, J=7.6 Hz, 1H), 6.84 (d, J=7.6 Hz, 1H), 6.80 (s, 1H), 3.84 (s, 3H), 3.70 (s, 3H), 3.62 (s, 2H), 3.43 (s, 2H), 2.26 (s, 6H).

### Step 3: Preparation of 2-[4-[(dimethylamino)methyl]-3-methoxy-phenyl]ethanol

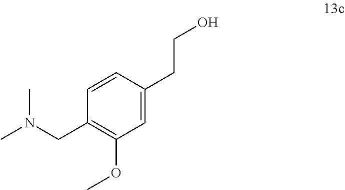

To a solution of methyl 2-[4-[(dimethylamino)methyl]-3-methoxy-phenyl]acetate (907.0 mg, Compound 13b, 3.82 mmol) in THF (10 mL) was added LiAlH₄ (290.0 mg, 7.64 mmol) under N₂ at 0° C. The mixture was stirred at 0° C. for 1 h. Then to the mixture was added H₂O (0.5 mL), NaOH (0.5 mL, 15% wt.) and H₂O (1.5 mL) at 0° C. to quench the reaction. The suspension was stirred at 25° C. for 10 min and filtered. Then the filtrate was dried over Na₂SO₄and filtered. The filtrate was concentrated in vacuo to afford 2-[4-[(dimethylamino)methyl]-3-methoxy-phenyl]ethanol (797.0 mg, Compound 13c) as a colorless oil, which was used directly to next step.

### Step 4: Preparation of 5-(2-bromoethyl)-2-[(dimethylamino)methyl]phenol (Compound 13d)

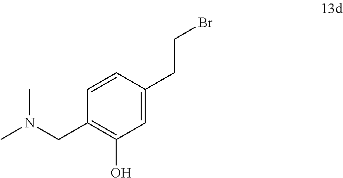

To a solution of 2-[4-[(dimethylamino)methyl]-3-methoxy-phenyl]ethanol (797.0 mg, Compound 13c, 3.81 mmol) in DCM (10 mL) was added BBr₃ (11.42 mL, 11.42 mmol, 1.0 M in DCM) dropwise at 0° C. The mixture was warmed to 25° C. and stirred at 25° C. for 6 h. Then the reaction was quenched with MeOH (20 mL) dropwise at 0° C. and concentrated in vacuo. The residue was purified by reversed prep-HPLC to afford 5-(2-bromoethyl)-2-[(dimethylamino) methyl]phenol (785.0 mg, Compound 13d) as a light yellow oil. MS obsd. (ESI⁺) [(M+H)⁺]: 257.9.

### Step 5: Preparation of 5-[2-[[4-amino-2-(ethoxymethyl)-6-methyl-1H-imidazo[4,5-c]pyridin-7-yl]sulfanyl]ethyl]-2-[(dimethylamino)methyl]phenol (Example 13)

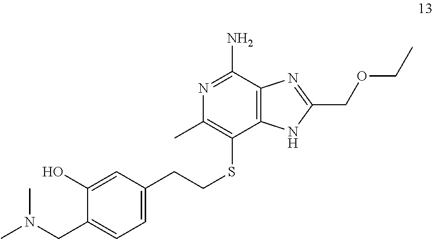 [SMILES: CCOCc1nc2c(N)nc(C)c(SCCc3ccc(CN(C)C)c(O)c3)c2[nH]1]

The title compound was prepared in analogy to Example 10, Step 3 by using 5-(2-bromoethyl)-2-[(dimethylamino)methyl]phenol (Compound 13d) instead of 4-(2-bromoethyl)benzaldehyde (Compound 10b). 5-[2-[[4-Amino-2-(ethoxymethyl)-6-methyl-1H-imidazo[4,5-c]pyridin-7-yl]sulfanyl]ethyl]-2-[(dimethylamino)methyl]phenol (4.19 mg, Example 13) was obtained as a light yellow solid. MS obsd. (ESI⁺) [(M+H)⁺]: 416.1. ¹H NMR (400 MHZ, CD₃OD) δ ppm: 7.13 (d, J=8.0 Hz, 1H), 6.74-6.65 (m, 2H), 4.73 (s, 2H), 4.22 (s, 2H), 3.71-3.62 (m, 2H), 3.10 (t, J=7.6 Hz, 2H), 2.87 (t, J=7.6 Hz, 2H), 2.81 (s, 6H), 2.66 (s, 3H), 1.26 (t, J=6.8 Hz, 3H).

### Example 14

### 2-butyl-7-[4-[(dimethylamino)methyl]phenyl]sulfanyl-6-methyl-1H-imidazo[4,5-c]pyridin-4-amine

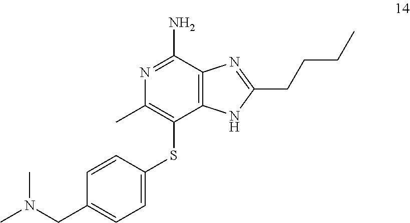 [SMILES: CCCCc1nc2c(N)nc(C)c(Sc3ccc(CN(C)C)cc3)c2[nH]1]

### Step 1: Preparation of N-[4-amino-2-[bis[(2,4-dimethoxyphenyl)methyl]amino]-6-methyl-3-pyridyl]pentanamide (Compound 14a)

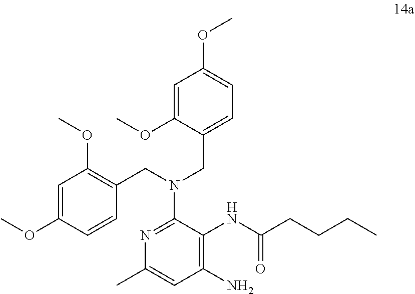 [SMILES: CCCCC(=O)Nc1c(N)cc(C)nc1N(Cc1ccc(OC)cc1OC)Cc1ccc(OC)cc1OC]

To a mixture of N2,N2-bis[(2,4-dimethoxyphenyl)methyl]-6-methyl-pyridine-2,3,4-triamine (2.0 g, Compound 1c, 4.56 mmol) and DIEA (1.18 g, 9.12 mmol) in DCM (20 mL) was added valeryl chloride (0.66 g, 5.47 mmol) at 0° C. The mixture was stirred at 25° C. for 12 h. Then the mixture was diluted with water (100 mL) and extracted with DCM (2×100 mL). The organic layer was washed with brine (100 mL), dried over Na₂SO₄ and filtered. The filtrate was concentrated in vacuo to afford N-[4-amino-2-[bis[(2,4-dimethoxyphenyl)methyl]amino]-6-methyl-3-pyridyl]pentanamide (2.0 g, Compound 14a) as a light yellow oil.

### Step 2: Preparation of 2-butyl-N,N-bis[(2,4-dimethoxyphenyl)methyl]-6-methyl-1H-imidazo[4,5-c]pyridin-4-amine (Compound 14b)

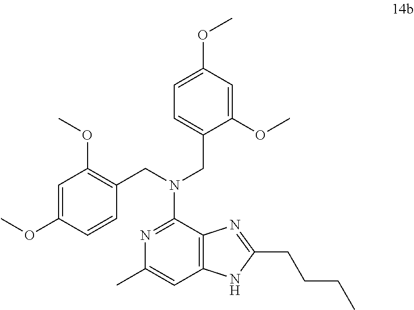 [SMILES: CCCCc1nc2c(N(Cc3ccc(OC)cc3OC)Cc3ccc(OC)cc3OC)nc(C)cc2[nH]1]

To a solution of N-[4-amino-2-[bis[(2,4-dimethoxyphenyl)methyl]amino]-6-methyl-3-pyridyl]pentanamide (2.38 g, Compound 14a, 4.56 mmol) in ethanol (20 mL) and water (10 mL) was added NaOH (1.85 g, 45.61 mmol) at 20° C. The mixture was stirred at 80° C. for 2 h. The reaction mixture was concentrated in vacuo. Then the residue was diluted with H₂O (100 mL) and extracted with DCM (3×100 mL). The filtrate was concentrated in vacuo and the residue was purified by reversed prep-HPLC to afford 2-butyl-N,N-bis[(2,4-dimethoxyphenyl)methyl]-6-methyl-1H-imidazo[4,5-c]pyridin-4-amine (1.4 g, Compound 14b) as a yellow solid. MS obsd. (ESI⁺) [(M+H)⁺]: 505.3.

### Step 3: Preparation of 2-butyl-6-methyl-1H-imidazo[4,5-c]pyridin-4-amine (Compound 14c)

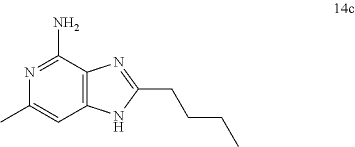

A solution of 2-butyl-N,N-bis[(2,4-dimethoxyphenyl)methyl]-6-methyl-1H-imidazo[4,5-c]pyridin-4-amine (1.4 g, Compound 14b, 2.77 mmol) and TFA (3.0 mL) in DCM (3.0 mL) was stirred at 20° C. for 4 h. The reaction mixture was concentrated in vacuo. The residue was purified by reversed prep-HPLC to afford 2-butyl-6-methyl-1H-imidazo[4,5-c]pyridin-4-amine (0.56 g, Compound 14c) as a white solid.

### Step 4: Preparation of 2-butyl-7-iodo-6-methyl-1H-imidazo[4,5-c]pyridin-4-amine (Compound 14d)

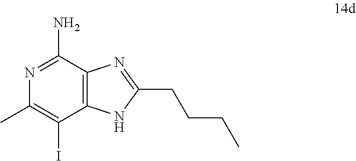 [SMILES: CCCCc1nc2c(N)nc(C)c(I)c2[nH]1]

To a solution of 2-butyl-6-methyl-1H-imidazo[4,5-c]pyridin-4-amine (0.57 g, Compound 14c, 2.77 mmol) in acetic acid (6.0 mL) was added N-iodosuccinimide (1.25 g, 5.55 mmol) at 20° C. The mixture was stirred at 20° C. for 2 h, and then concentrated in vacuo. The residue was purified by reversed prep-HPLC to afford 2-butyl-7-iodo-6-methyl-1H-imidazo[4,5-c]pyridin-4-amine (0.43 g, Compound 14d) as an orange oil. MS obsd. (ESI⁺) [(M+H)⁺]: 330.9. ¹H NMR (400 MHz, DMSO-d₆) δ ppm: 13.12 (s, 1H), 11.07 (s, 1H), 8.167 (s, 1H), 2.86 (t, J=7.6 Hz, 2H), 2.56 (s, 3H), 1.78-1.69 (m, 2H), 1.40-1.30 (m, 2H), 0.91 (t, J=7.2 Hz, 3H).

### Step 5: Preparation of 2-butyl-7-[4-[(dimethylamino)methyl]phenyl]sulfanyl-6-methyl-1H-imidazo[4,5-c]pyridin-4-amine (Example 14)

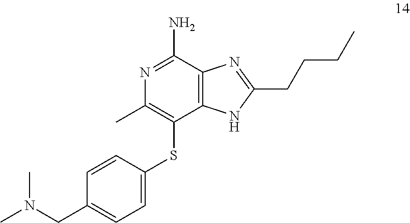 [SMILES: CCCCc1nc2c(N)nc(C)c(Sc3ccc(CN(C)C)cc3)c2[nH]1]

The title compound was prepared in analogy to Example 5, Step 3 by using 2-butyl-7-iodo-6-methyl-1H-imidazo[4,5-c]pyridin-4-amine (Compound 14d) instead of 2-(ethoxymethyl)-7-iodo-6-methyl-1H-imidazo[4,5-c]pyridin-4-amine (Compound 1g) and 4-[(dimethylamino)methyl]benzenethiol (Compound 1k) instead of N-ethyl-N-[[4-[[4-[[ethyl(propyl)amino]methyl]phenyl]disulfanyl]phenyl]methyl]propan-1-amine (Compound 5b). 2-Butyl-7-[4-[(dimethylamino)methyl]phenyl]sulfanyl-6-methyl-1H-imidazo[4,5-c]pyridin-4-amine (30.1 mg, Example 14) was obtained as a white solid. MS obsd. (ESI⁺) [(M+H)⁺]: 370.2. ¹H NMR (400 MHz, CD₃OD) δ ppm: 7.17 (d, J=8.4 Hz, 2H), 6.95 (d, J=8.4 Hz, 2H), 3.44 (s, 2H), 2.82 (t, J=7.6 Hz, 2H), 2.50 (s, 3H), 2.23 (s, 6H), 1.83-1.71 (m, 2H), 1.47-1.28 (m, 2H), 0.94 (t, J=7.2 Hz, 3H).

### Example 15

### 2-butyl-7-[2-[4-[(dimethylamino)methyl]phenyl]ethylsulfanyl]-6-methyl-1H-imidazo[4,5-c]pyridin-4-amine

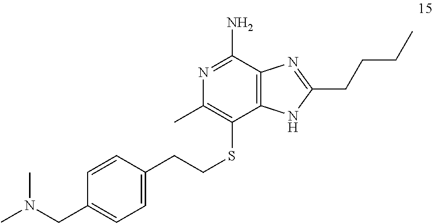 [SMILES: CCCCc1nc2c(N)nc(C)c(SCCc3ccc(CN(C)C)cc3)c2[nH]1]

### Step 1: Preparation of 4-[2-[(4-amino-2-butyl-6-methyl-1H-imidazo[4,5-c]pyridin-7-yl)sulfanyl]ethyl]benzaldehyde (Compound 15a)

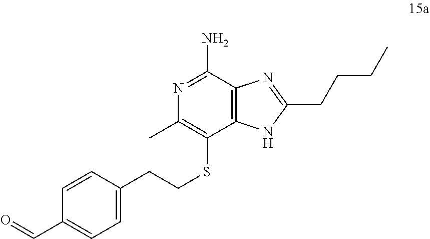 [SMILES: CCCCc1nc2c(N)nc(C)c(SCCc3ccc(C=O)cc3)c2[nH]1]

Compound 15a was prepared in analogy to Example 13, Step 5 by using 2-butyl-7-iodo-6-methyl-1H-imidazo[4,5-c]pyridin-4-amine (Compound 14d) instead of 2-(ethoxymethyl)-7-iodo-6-methyl-1H-imidazo[4,5-c]pyridin-4-amine (Compound 1g) and 4-(2-bromoethyl)benzaldehyde (Compound 10b) instead of 5-(2-bromoethyl)-2-[(dimethylamino)methyl]phenol (Compound 13d). 4-[2-[(4-Amino-2-butyl-6-methyl-1H-imidazo[4,5-c]pyridin-7-yl)sulfanyl]ethyl]benzaldehyde (45.0 mg, Compound 15a) was obtained as a yellow solid.

### Step 2: Preparation of 2-butyl-7-[2-[4-[(dimethylamino)methyl]phenyl]ethylsulfanyl]-6-methyl-1H-imidazo[4,5-c]pyridin-4-amine (Example 15)

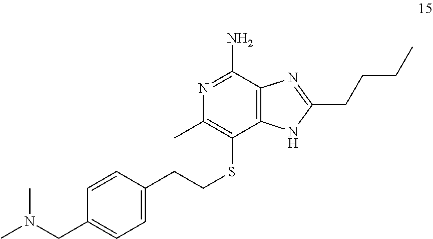 [SMILES: CCCCc1nc2c(N)nc(C)c(SCCc3ccc(CN(C)C)cc3)c2[nH]1]

The title compound was prepared in analogy to Example 1, Step 10 by using 4-[2-[(4-amino-2-butyl-6-methyl-1H-imidazo[4,5-c]pyridin-7-yl)sulfanyl]ethyl]benzaldehyde (Compound 15a) instead of S-(4-formylphenyl) N,N-dimethylcarbamothioate (Compound 1i). 2-Butyl-7-[2-[4-[(dimethylamino)methyl]phenyl]ethylsulfanyl]-6-methyl-1H-imidazo[4,5-c]pyridin-4-amine (5.2 mg, Example 15) was obtained as a light red solid. MS obsd. (ESI⁺) [(M+H)⁺]: 398.2. ¹H NMR (400 MHz, CD₃OD) δ ppm: 7.15 (d, J=8.0 Hz, 2H), 7.06 (d, J=8.0 Hz, 2H), 3.42 (s, 2H), 2.96 (t, J=8.0 Hz, 2H), 2.88 (t, J=7.6 Hz, 2H), 2.79 (t, J=8.0 Hz, 2H), 2.55 (s, 3H), 2.21 (s, 6H), 1.87-1.77 (m, 2H), 1.48-1.38 (m, 2H), 0.98 (t, J=7.6 Hz, 3H).

### Example 16

### 6-[[4-amino-2-(ethoxymethyl)-6-methyl-1H-imidazo[4,5-c]pyridin-7-yl]sulfanyl]-3-[(dimethylamino)methyl]-2-methyl-phenol

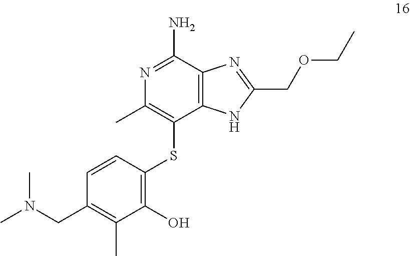 [SMILES: CCOCc1nc2c(N)nc(C)c(Sc3ccc(CN(C)C)c(C)c3O)c2[nH]1]

### Step 1: Preparation of 2-methoxy-4-(methoxymethyl)phenol (Compound 16a)

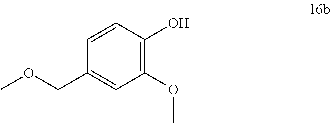

To a solution of 4-hydroxy-3-methoxybenzyl alcohol (10.0 g, 64.87 mmol) in methanol (150 mL) was added p-toluenesulfonic acid (0.56 g, 3.24 mmol) at 20° C. The reaction mixture was stirred at 20° C. for 6 h. The mixture was concentrated in vacuo, diluted with sat. NaHCO₃ (200 mL) and extracted with EA (3×200 mL). The organic layer was washed with brine (3×200 mL), dried over Na₂SO₄ and concentrated in vacuo. The residue was purified by silica gel column to afford 2-methoxy-4-(methoxymethyl)phenol (10.0 g, Compound 16a) as a yellow oil. ¹H NMR (400 MHz, CDCl₃) δ ppm: 6.89-6.81 (m, 3H), 5.76 (s, 1H), 4.38 (s, 2H), 3.89 (s, 3H), 3.37 (s, 3H).

### Step 2: Preparation of 2-methoxy-4-(methoxymethyl)-3-methyl-phenol (Compound 16b)

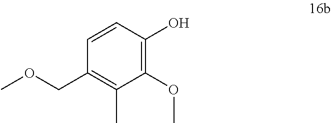

To a solution of 2-methoxy-4-(methoxymethyl)phenol (9.0 g, Compound 16a, 53.51 mmol) in THF (90 mL) was slowly added n-BuLi (64.21 mL, 160.53 mmol) at −10° C. After the addition was completed, the reaction was stirred at 0° C. for 2.5 h. The reaction was then cooled to −10° C. before adding iodomethane (11.56 mL, 187.29 mmol). The reaction mixture was stirred at 0° C. for 1.5 h. Then the reaction was quenched with water (100 mL) and acidified to pH=7 with aq. HCl (1.0 M). The mixture was extracted with EA (3×150 mL). The organic layer was washed with brine (3×100 mL) and concentrated in vacuo. The residue was purified by silica gel column to afford 2-methoxy-4-(methoxymethyl)-3-methyl-phenol (6.0 g, Compound 16b) as a light yellow oil.

### Step 3: Preparation of 4-hydroxy-3-methoxy-2-methyl-benzaldehyde (Compound 16c)

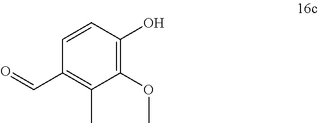

To a solution of 2-methoxy-4-(methoxymethyl)-3-methyl-phenol (6.0 g, Compound 16b, 32.93 mmol) in DCM (75 mL) and water (7.5 mL) was slowly added DDQ (7.47 g, 32.93 mmol) at 20° C. The reaction mixture was stirred at 20° C. for 16 h. The reaction was filtered and filtrate was concentrated in vacuo. The residue was purified by silica gel column to afford 4-hydroxy-3-methoxy-2-methyl-benzaldehyde (4.5 g, Compound 16c) as a white solid.

### Step 4: Preparation of O-(4-formyl-2-methoxy-3-methyl-phenyl) N,N-dimethylcarbamothioat (Compound 16d)

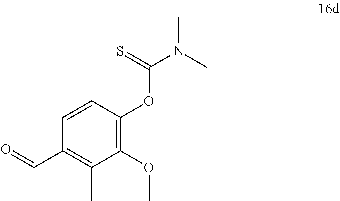 [SMILES: COc1c(OC(=S)N(C)C)ccc(C=O)c1C]

To a solution of 4-hydroxy-3-methoxy-2-methyl-benzaldehyde (1.0 g, Compound 16c, 6.02 mmol) in DMF (10 mL) were added DABCO (2.7 g, 24.07 mmol) and dimethylthiocarbamoyl chloride (1.49 g, 12.04 mmol) at 20° C. The mixture was stirred at 20° C. for 12 h. Then the mixture was diluted with H₂O (100 mL) and extracted with EA (3×100 mL). The organic layer was washed with brine (3×80 mL), dried over Na₂SO₄, filtered and concentrated in vacuo. The residue was purified by re-crystallization from MeOH (10 mL) to afford O-(4-formyl-2-methoxy-3-methyl-phenyl) N,N-dimethylcarbamothioate (500.0 mg, Compound 16d) as a light yellow solid. MS obsd. (ESI⁺) [(M+H)⁺]: 254.2.

### Step 5: Preparation of S-(4-formyl-2-methoxy-3-methyl-phenyl) N,N-dimethylcarbamothioate (Compound 16e)

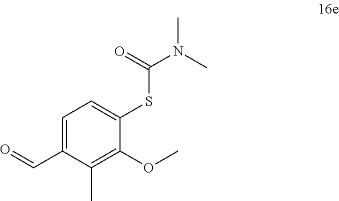 [SMILES: COc1c(SC(=O)N(C)C)ccc(C=O)c1C]

A solution of O-(4-formyl-2-methoxy-3-methyl-phenyl) N,N-dimethylcarbamothioate (2.2 g, Compound 16d, 8.68 mmol) in Ph₂O (25.0 mL) was stirred at 240° C. for 2 h. The reaction turned black. The mixture was purified by silica gel column to afford S-(4-formyl-2-methoxy-3-methyl-phenyl) N,N-dimethylcarbamothioate (1.00 g, Compound 16e) as a light yellow solid. ¹H NMR (400 MHz, CDCl₃) δ ppm: 10.27 (s, 1H), 7.62-7.55 (m, 2H), 3.80 (s, 3H), 3.17 (s, 3H), 3.11 (s, 3H), 2.64 (s, 3H).

### Step 6: Preparation of S-[4-[(dimethylamino)methyl]-2-methoxy-3-methyl-phenyl]N,N-dimethylcarbamothioate (Compound 16f)

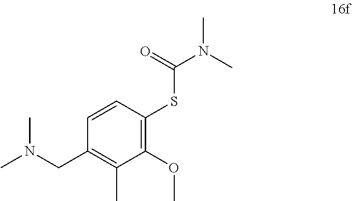

To a solution of S-(4-formyl-2-methoxy-3-methyl-phenyl) N,N-dimethylcarbamothioate (1.0 g, Compound 16e, 3.95 mmol) in methanol (10 mL) were added NaOAc (0.97 g, 11.84 mmol) and dimethylamine hydrochloride (0.97 g, 11.84 mmol) at 20° C. The reaction mixture was stirred at 20° C. for 12 h. Then to the mixture was added NaBH(OAc)₃ (3.35 g, 15.79 mmol) at 20° C. The reaction mixture was stirred at 20° C. for 2 h, then concentrated in vacuo and the residue was purified by reversed prep-HPLC to afford S-[4-[(dimethylamino)methyl]-2-methoxy-3-methyl-phenyl]N,N-dimethylcarbamothioate (850.0 mg, Compound 16f) as a white solid. MS obsd. (ESI⁺) [(M+H)⁺]: 283.2.

### Step 7: Preparation of S-[4-[(dimethylamino)methyl]-2-hydroxy-3-methyl-phenyl]N,N-dimethylcarbamothioate (Compound 16g)

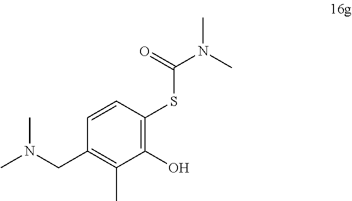

To a solution of S-[4-[(dimethylamino)methyl]-2-methoxy-3-methyl-phenyl]N,N-dimethylcarbamothioate (800.0 mg, Compound 16f, 2.83 mmol) in DCM (10 mL) was added BBr₃ (7.08 mL, 7.08 mmol) dropwise at 0° C. The mixture was stirred at 25° C. for 3 h. The reaction was quenched with MeOH (10 mL) dropwise at 0° C. The reaction mixture was concentrated in vacuo and the residue was purified by reversed prep-HPLC to afford S-[4-[(dimethylamino)methyl]-2-hydroxy-3-methyl-phenyl]N,N-dimethylcarbamothioate (650.0 mg, Compound 16g) as a white solid.

### Step 8: Preparation of 3-[(dimethylamino)methyl]-2-methyl-6-sulfanyl-phenol (Compound 16h)

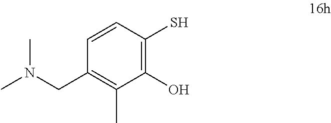

To a solution of S-[4-[(dimethylamino)methyl]-2-hydroxy-3-methyl-phenyl]N,N-dimethylcarbamothioate (590.0 mg, Compound 16g, 1.88 mmol) in THF (10 mL) was added LiAlH₄ (356.08 mg, 9.38 mmol) at 0° C. under N₂. The mixture was stirred at 20° C. for 0.5 h. The mixture was quenched with H₂O (0.4 mL), NaOH (0.4 mL, 10 M in H₂O), and H₂O (1.2 mL) at 0° C. The reaction mixture was stirred at 20° C. for 2 h. The mixture was filtered and the filtrate was concentrated in vacuo. The residue was purified by reversed prep-HPLC to afford 3-[(dimethylamino)methyl]-2-methyl-6-sulfanyl-phenol (250.0 mg, Compound 16h) as a light brown solid. MS obsd. (ESI⁺) [(M+H)⁺]: 198.3.

### Step 9: Preparation of 6-[[4-amino-2-(ethoxymethyl)-6-methyl-1H-imidazo[4,5-c]pyridin-7-yl]sulfanyl]-3-[(dimethylamino)methyl]-2-methyl-phenol (Example 16)

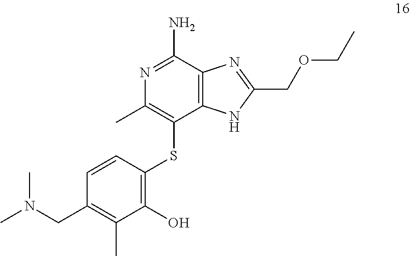 [SMILES: CCOCc1nc2c(N)nc(C)c(Sc3ccc(CN(C)C)c(C)c3O)c2[nH]1]

To a solution of 2-(ethoxymethyl)-7-iodo-6-methyl-1H-imidazo[4,5-c]pyridin-4-amine (80.0 mg, Compound 1g, 0.24 mmol) in DMSO (3.0 mL) were added 3-[(dimethylamino)methyl]-2-methyl-6-sulfanyl-phenol (94.56 mg, Compound 16 h, 0.24 mmol), Cu₂S (76.67 mg, 0.48 mmol), Fe (40.32 mg, 0.72 mmol) and K₂CO₃ (133.16 mg, 0.96 mmol) at 20° C. The suspension was degassed in vacuo and purged with N₂ three times. The reaction mixture was stirred at 100° C. for 12 h under N₂. The reaction mixture was filtered and the filtrate was purified by prep-HPLC to afford 6-[[4-amino-2-(ethoxymethyl)-6-methyl-1H-imidazo[4,5-c]pyridin-7-yl]sulfanyl]-3-[(dimethylamino)methyl]-2-methyl-phenol (3.74 mg, Example 16) as a white solid. MS obsd. (ESI⁺) [(M+H)⁺]: 402.4. ¹H NMR (400 MHz, CD₃OD) δ ppm: 6.73 (d, J=8.4 Hz, 1H), 6.52 (d, J=8.4 Hz, 1H), 4.65 (s, 2H), 3.98 (br s, 2H), 3.63-3.58 (m, 2H), 2.62 (s, 6H), 2.53 (s, 3H), 2.31 (s, 3H), 1.22 (t, J=6.8 Hz, 3H).

### Example 17

### 2-[[4-amino-2-(ethoxymethyl)-6-methyl-1H-imidazo[4,5-c]pyridin-7-yl]sulfanyl]-5-(diethylaminomethyl)phenol

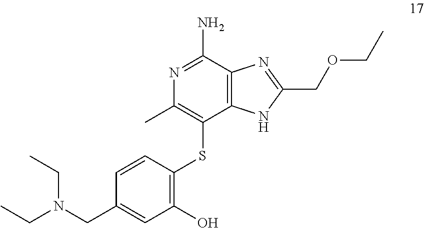 [SMILES: CCOCc1nc2c(N)nc(C)c(Sc3ccc(CN(CC)CC)cc3O)c2[nH]1]

### Step 1: Preparation of 2-ethylhexyl 3-(4-formyl-2-hydroxy-phenyl)sulfanylpropanoate (Compound 17a)

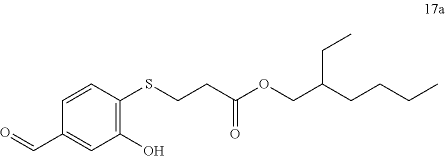 [SMILES: CCCCC(CC)COC(=O)CCSc1ccc(C=O)cc1O]

To a solution of 3-hydroxy-4-iodo-benzaldehyde (10.0 g, 40.32 mmol) in 1,4-dioxane (100 mL) were added 3-mercaptopropionic acid 2-ethylhexyl ester (13.21 g, 60.48 mmol), DIEA (15.63 g, 120.96 mmol), Pd₂(dba)₃ (1.17 g, 2.02 mmol) and XantPhos (3.69 g, 4.03 mmol) at 25° C. The suspension was degassed in vacuo and purged with N2 three times. The suspension was heated to 110° C. and stirred at 110° C. for 16 h under N₂. Then the reaction mixture was filtered through celite and washed with MeOH (100 mL). The filtrate was concentrated in vacuo. The residue was purified by silica gel column and concentrated in vacuo to afford 2-ethylhexyl 3-(4-formyl-2-hydroxy-phenyl)sulfanylpropanoate (7.6 g, Compound 17a) as a light yellow oil. MS obsd. (ESI⁺) [(M+H)⁺]: 337.1.

### Step 2: Preparation of 2-ethylhexyl 3-[4-(diethylaminomethyl)-2-hydroxy-phenyl]sulfanylpropanoate (Compound 17b)

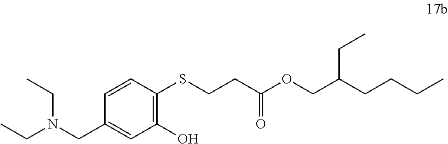 [SMILES: CCCCC(CC)COC(=O)CCSc1ccc(CN(CC)CC)cc1O]

To a solution of 2-ethylhexyl 3-(4-formyl-2-hydroxy-phenyl)sulfanylpropanoate (700.0 mg, Compound 17a, 2.07 mmol) in methanol (10 mL) was added diethylamine (1.51 g, 20.68 mmol) at 20° C. The reaction mixture was stirred at 20° C. for 12 h, then NaBH(OAc)₃ (4.38 g, 20.68 mmol) was added at 20° C. The reaction mixture was stirred at 20° C. for 2 h. The reaction mixture was concentrated in vacuo and the residue was purified by reversed prep-HPLC to afford 2-ethylhexyl 3-[4-(diethylaminomethyl)-2-hydroxy-phenyl]sulfanylpropanoate (150.0 mg, Compound 17b) as a light yellow oil. MS obsd. (ESI⁺) [(M+H)⁺]: 394.0.

### Step 3: Preparation of 2-[[4-amino-2-(ethoxymethyl)-6-methyl-1H-imidazo[4,5-c]pyridin-7-yl]sulfanyl]-5-(diethylaminomethyl)phenol (Example 17)

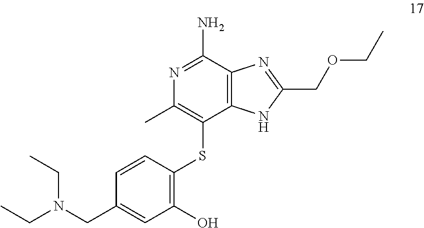 [SMILES: CCOCc1nc2c(N)nc(C)c(Sc3ccc(CN(CC)CC)cc3O)c2[nH]1]

The title compound was prepared in analogy to Example 16, Step 9 by using 2-ethylhexyl 3-[4-(diethylaminomethyl)-2-hydroxy-phenyl]sulfanylpropanoate (Compound 17b) instead of 3-[(dimethylamino)methyl]-2-methyl-6-sulfanyl-phenol (Compound 16h). 2-[[4-Amino-2-(ethoxymethyl)-6-methyl-1H-imidazo[4,5-c]pyridin-7-yl]sulfanyl]-5-(diethylaminomethyl)phenol (25.97 mg, Example 17) was obtained as a white solid. MS obsd. (ESI⁺) [(M+H)⁺]: 416.2. ¹H NMR (400 MHz, CD₃OD) δ ppm: 6.80 (d, J=1.6 Hz, 1H), 6.60 (d, J=8.0 Hz, J=1.6 Hz, 1H), 6.45 (d, J=8.0 Hz, 1H), 4.63 (s, 2H), 3.62-3.57 (m, 2H), 3.56 (s, 2H), 2.60-2.54 (m, 4H), 2.53 (s, 3H), 1.21 (t, J=6.8 Hz, 3H), 1.06 (t, J=7.2 Hz, 6H).

### Example 18

### 7-[[4-[(dimethylamino)methyl]-1H-indol-7-yl]sulfanyl]-2-(ethoxymethyl)-6-methyl-1H-imidazo[4,5-c]pyridin-4-amine

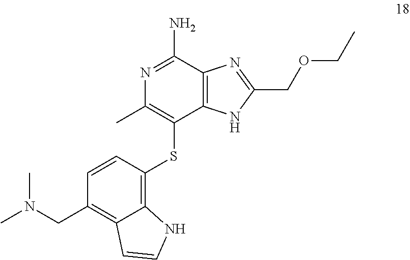 [SMILES: CCOCc1nc2c(N)nc(C)c(Sc3ccc(CN(C)C)c4cc[nH]c34)c2[nH]1]

### Step 1: Preparation of methyl 7-bromo-1H-indole-4-carboxylate (Compound 18a)

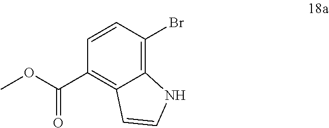

To a solution of methyl 4-bromo-3-nitrobenzoate (10.0 g, 38.46 mmol) in THF (100 mL) was added vinylmagnesium bromide (115.37 mL, 115.37 mmol) dropwise at −40° C. The reaction mixture was stirred at −40° C. for 2 h. Then the reaction was quenched with sat. NH₄Cl (200 mL) dropwise at −40° C. The mixture was warmed to 25° C. and stirred at this temperature for 1 h, then extracted with EA (2×100 mL). The combined organic layer was washed with brine (100 mL), dried over Na₂SO₄, filtered and concentrated in vacuo. The residue was purified by silica gel column and concentrated in vacuo to afford methyl 7-bromo-1H-indole-4-carboxylate (2.8 g, Compound 18a) as a light yellow solid. ¹H NMR (400 MHz, CDCl₃) δ ppm: 8.55 (s, 1H), 7.82 (d, J=8.0 Hz, 1H), 7.44-7.42 (m, 2H), 7.29-7.27 (m, 1H), 3.99 (s, 3H).

### Step 2: Preparation of (7-bromo-1H-indol-4-yl)methanol (Compound 18b)

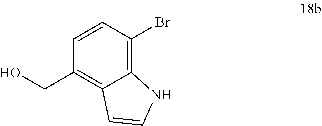 [SMILES: OCc1ccc(Br)c2[nH]ccc12]

To a solution of methyl 7-bromo-1H-indole-4-carboxylate (2.80 g, Compound 18a, 11.02 mmol) in THF (28 mL) was added LiAlH₄ (836.43 mg, 22.04 mmol) at 0° C. The reaction mixture was stirred at 0° C. for 2 h. After 2 h, H₂O (1 mL) and NaOH (1 mL, 10 M in H₂O) were added dropwise at 0° C., then H₂O (3 mL) and Na₂SO₄ (30 g) were added. The reaction mixture was stirred at 20° C. for 2 h, then filtered through celite and washed with DCM/MeOH (100 mL, V/V=1/10). The filtrate was concentrated in vacuo and the residue was purified by reversed prep-HPLC to afford (7-bromo-1H-indol-4-yl)methanol (2.5 g, Compound 18b) as a light brown solid. ¹H NMR (400 MHz, CD₃OD) δ ppm: 7.32 (d, J=3.2 Hz, 1H), 7.25 (d, J=7.6 Hz, 1H), 6.97 (d, J=7.6 Hz, 1H), 6.67 (d, J=3.2 Hz, 1H), 4.86 (s, 2H).

### Step 3: Preparation of 7-bromo-1H-indole-4-carbaldehyde (Compound 18c)

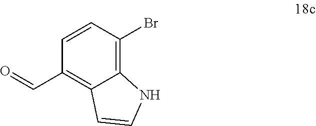 [SMILES: O=Cc1ccc(Br)c2[nH]ccc12]

To a solution of (7-bromo-1H-indol-4-yl)methanol (2.5 g, Compound 18b, 11.06 mmol) in DCM (30 mL) was added MnO₂ (14.42 g, 165.88 mmol) at 0° C. The reaction mixture was stirred at 25° C. for 12 h. The reaction mixture was filtered through celite, washed with DCM (100 mL). The filtrate was concentrated in vacuo. The residue was purified by silica gel column and concentrated in vacuo to afford 7-bromo-1H-indole-4-carbaldehyde (1.5 g, Compound 18c) as a light yellow solid. MS obsd. (ESI⁺) [(M+H)⁺]: 226.1.

### Step 4: Preparation of 2-ethylhexyl 3-[(4-formyl-1H-indol-7-yl)sulfanyl]propanoate (Compound 18d)

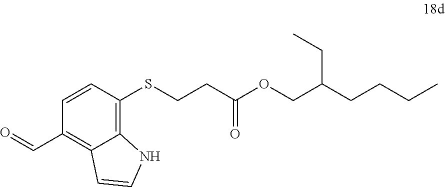 [SMILES: CCCCC(CC)COC(=O)CCSc1ccc(C=O)c2cc[nH]c12]

Compound 18d was prepared in analogy to Example 17, Step 1 by using 7-bromo-1H-indole-4-carbaldehyde (Compound 18c) instead of 3-hydroxy-4-iodo-benzaldehyde. 2-Ethylhexyl 3-[(4-formyl-1H-indol-7-yl)sulfanyl]propanoate (2.0 g, Compound 18d) was obtained as a light yellow oil. MS obsd. (ESI⁺) [(M+H)⁺]: 362.4.

### Step 5: Preparation of 2-ethylhexyl 3-[[4-[(dimethylamino)methyl]-1H-indol-7-yl]sulfanyl]propanoate (Compound 18e)

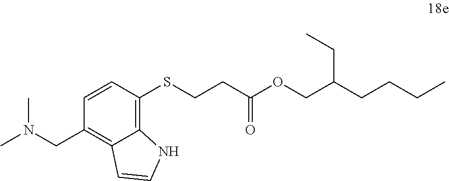 [SMILES: CCCCC(CC)COC(=O)CCSc1ccc(CN(C)C)c2cc[nH]c12]

Compound 18e was prepared in analogy to Example 1, Step to by using 2-ethylhexyl 3-[(4-formyl-1H-indol-7-yl)sulfanyl]propanoate2-ethylhexyl 3-[(4-formyl-1H-indol-7-yl)sulfanyl]propanoate (Compound 18d) instead of S-(4-formylphenyl) N,N-dimethylcarbamothioate (Compound 1i). 2-Ethylhexyl 3-[[4-[(dimethylamino)methyl]-1H-indol-7-yl]sulfanyl]propanoate (200.0 mg, Compound 18e) was obtained as a light brown oil.

### Step 6: Preparation of 7-[[4-[(dimethylamino)methyl]-1H-indol-7-yl]sulfanyl]-2-(ethoxymethyl)-6-methyl-1H-imidazo[4,5-c]pyridin-4-amine (Example 18)

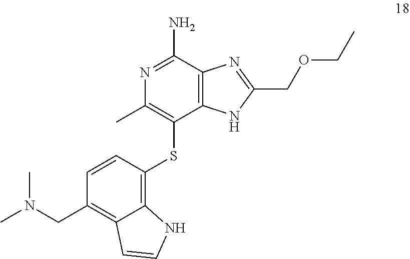 [SMILES: CCOCc1nc2c(N)nc(C)c(Sc3ccc(CN(C)C)c4cc[nH]c34)c2[nH]1]

The title compound was prepared in analogy to Example 16, Step 9 by using 2-ethylhexyl 3-[[4-[(dimethylamino)methyl]-1H-indol-7-yl]sulfanyl]propanoate (Compound 18e) instead of 3-[(dimethylamino)methyl]-2-methyl-6-sulfanyl-phenol (Compound 16h). 7-[[4-[(Dimethylamino)methyl]-1H-indol-7-yl]sulfanyl]-2-(ethoxymethyl)-6-methyl-1H-imidazo[4,5-c]pyridin-4-amine (15.12 mg, Example 18) was obtained as a white solid. MS obsd. (ESI⁺) [(M+H)⁺]: 411.3. ¹H NMR (400 MHz, CD₃OD) δ ppm: 7.34 (d, J=3.2 Hz, 1H), 6.82 (d, J=7.6 Hz, 1H), 6.64 (d, J=3.2 Hz, 1H), 6.47 (d, J=7.6 Hz, 1H), 4.64 (s, 2H), 3.72-3.66 (m, 2H), 3.62-3.55 (m, 2H), 2.53 (s, 3H), 2.25 (s, 6H), 1.20 (t, J=7.2 Hz, 3H).

### Example 19

### 7-[[4-[(dimethylamino)methyl]-1H-indazol-7-yl]sulfanyl]-2-(ethoxymethyl)-6-methyl-1H-imidazo[4,5-c]pyridin-4-amine

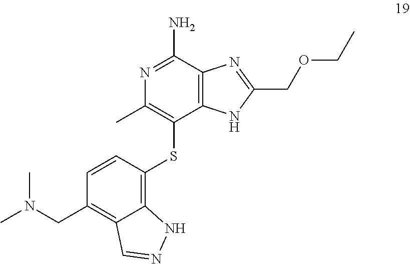 [SMILES: CCOCc1nc2c(N)nc(C)c(Sc3ccc(CN(C)C)c4cn[nH]c34)c2[nH]1]

### Step 1: Preparation of 6-bromo-2-fluoro-3-iodo-benzaldehyde (Compound 19a)

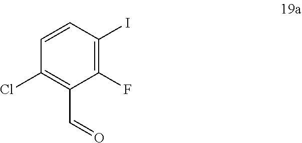 [SMILES: O=Cc1c(Cl)ccc(I)c1F]

To a solution of 1-bromo-3-fluoro-4-iodobenzene (10.0 g, 33.23 mmol) in THF (100 mL) was added LDA (19.94 mL, 39.88 mmol) dropwise at −78° C. The mixture was stirred at −78° C. for 0.5 h. Then to the mixture was added DMF (7.29 g, 99.7 mmol) dropwise at −78° C. The mixture was stirred at −78° C. for 2 h. The mixture was quenched with sat NH₄Cl (200 mL) at −78° C. The mixture was warmed to 25° C. and extracted with EtOAc (2×400 mL). The organic layer was washed with brine (2×200 mL), dried over Na₂SO₄, filtered and concentrated in vacuo. The residue was purified by silica gel column and concentrated in vacuo to afford 6-bromo-2-fluoro-3-iodo-benzaldehyde (7.6 g, Compound 19a) as a yellow solid.

### Step 2: Preparation of 4-bromo-7-iodo-1H-indazole (Compound 19b)

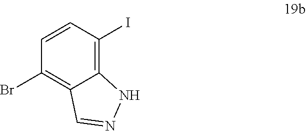 [SMILES: Brc1ccc(I)c2[nH]ncc12]

To a solution of 6-bromo-2-fluoro-3-iodo-benzaldehyde (6.6 g, Compound 19a, 20.07 mmol) in DMSO (80 mL) was added hydrazine hydrate (7.69 g, 150.5 mmol) at 20° C. The mixture was stirred at 130° C. for 6 h. The mixture was diluted with water (150 mL) and extracted with EA (3×300 mL). The combined organic layer was washed with brine (3×100 mL), dried over Na₂SO₄, filtered and concentrated in vacuo. The residue was purified by silica gel column and concentrated in vacuo to afford 4-bromo-7-iodo-1H-indazole (5.90 g, Compound 19b) as a white solid. MS obsd. (ESI⁺) [(M+H)⁺]: 324.9.

### Step 3: Preparation 2-ethylhexyl 3-[(4-bromo-1H-indazol-7-yl)sulfanyl]propanoate (Compound 19c)

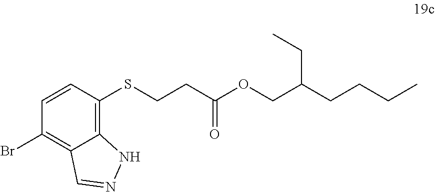 [SMILES: CCCCC(CC)COC(=O)CCSc1ccc(Br)c2cn[nH]c12]

Compound 19c was prepared in analogy to Example 17, Step 1 by using 4-bromo-7-iodo-1H-indazole (Compound 19b) instead of 3-hydroxy-4-iodo-benzaldehyde. 2-Ethylhexyl 3-[(4-bromo-1H-indazol-7-yl)sulfanyl]propanoate (1.60 g, Compound 19c) was obtained as a yellow oil. MS obsd. (ESI⁺) [(M+H)⁺]: 413.1.

### Step 4: Preparation of 4-bromo-7-[(4-bromo-1H-indazol-7-yl)disulfanyl]-1H-indazole (Compound 19d)

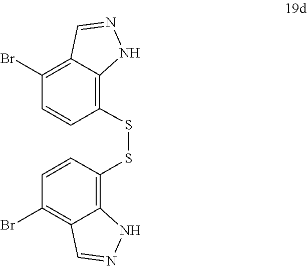 [SMILES: Brc1ccc(SSc2ccc(Br)c3cn[nH]c23)c2[nH]ncc12]

To a solution of 2-ethylhexyl 3-[(4-bromo-1H-indazol-7-yl)sulfanyl]propanoate (800.0 mg, Compound 19c, 1.94 mmol) in THF (10.0 mL) was added t-BuOK (5.81 mL, 5.81 mmol, 1.0 M in THF) dropwise at −78° C. The mixture was stirred at −78° C. for 1 h, then warmed to 25° C. and adjusted pH=5 with aq. HCl (1.0 M). The mixture was concentrated in vacuo. The residue was purified by silica gel column and concentrated in vacuo to afford 4-bromo-7-[(4-bromo-1H-indazol-7-yl)disulfanyl]-1H-indazole (170.0 mg, Compound 19d) as a light yellow solid. MS obsd. (ESI⁺) [(M+H)⁺]: 456.9.

### Step 5: Preparation of 7-[(4-bromo-1H-indazol-7-yl)sulfanyl]-2-(ethoxymethyl)-6-methyl-1H-imidazo[4,5-c]pyridin-4-amine (Compound 19e)

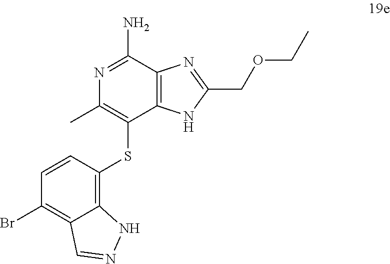 [SMILES: CCOCc1nc2c(N)nc(C)c(Sc3ccc(Br)c4cn[nH]c34)c2[nH]1]

Compound 19e was prepared in analogy to Example 16, Step 9 by using 4-bromo-7-[(4-bromo-1H-indazol-7-yl)disulfanyl]-1H-indazole (Compound 19d) instead of 3-[(dimethylamino)methyl]-2-methyl-6-sulfanyl-phenol (Compound 16h). 7-[(4-Bromo-1H-indazol-7-yl)sulfanyl]-2-(ethoxymethyl)-6-methyl-1H-imidazo[4,5-c]pyridin-4-amine (60.0 mg, Compound 19e) was obtained as a light brown foam. MS obsd. (ESI⁺) [(M+H)⁺]: 433.2.

### Step 6: Preparation of 7-[[4-[(dimethylamino)methyl]-LH-indazol-7-yl]sulfanyl]-2-(ethoxymethyl)-6-methyl-LH-imidazo[4,5-c]pyridin-4-amine (Example 19)

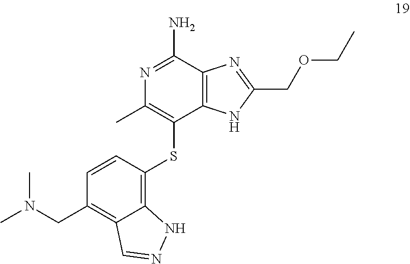 [SMILES: CCOCc1nc2c(N)nc(C)c(Sc3ccc(CN(C)C)c4cn[nH]c34)c2[nH]1]

To a solution of 7-[(4-bromo-1H-indazol-7-yl)sulfanyl]-2-(ethoxymethyl)-6-methyl-1H-imidazo[4,5-c]pyridin-4-amine (50.0 mg, Compound 19e, 0.12 mmol) in 1,4-dioxane (1.0 mL) and water (0.2 mL) were added potassium dimethylaminomethyltrifluoroborate (57.12 mg, 0.35 mmol), KOH (32.37 mg, 0.58 mmol) and XPhos Pd G4 (19.86 mg, 0.02 mmol) at 25° C. The suspension was degassed in vacuo and purged with nitrogen three times. The mixture was stirred at 100° C. for 12 h under N₂ balloon, then filtered and the filtrate was concentrated in vacuo. The residue was purified by reversed prep-HPLC and lyophilized to afford 7-[[4-[(dimethylamino)methyl]-1H-indazol-7-yl]sulfanyl]-2-(ethoxymethyl)-6-methyl-1H-imidazo[4,5-c]pyridin-4-amine (8.0 mg, Example 19) as a white solid. MS obsd. (ESI⁺) [(M+H)⁺]: 412.2. ¹H NMR (400 MHz, CD₃OD) δ ppm: 8.26 (s, 1H), 6.88 (d, J=7.2 Hz, 1H), 6.65-6.44 (m, 1H), 4.63 (s, 2H), 3.72 (s, 2H), 3.63-3.53 (m, 2H), 2.55 (s, 3H), 2.25 (s, 6H), 1.20 (t, J=7.2 Hz, 3H).

### Example 20

### 7-[2-amino-4-[(dimethylamino)methyl]phenyl]sulfanyl-2-(ethoxymethyl)-6-methyl-LH-imidazo[4,5-c]pyridin-4-amine

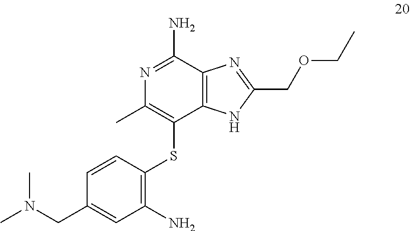 [SMILES: CCOCc1nc2c(N)nc(C)c(Sc3ccc(CN(C)C)cc3N)c2[nH]1]

### Step 1: Preparation of 2-ethylhexyl 3-(4-formyl-2-nitro-phenyl)sulfanylpropanoate (Compound 20a)

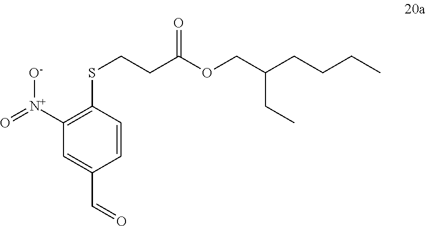 [SMILES: CCCCC(CC)COC(=O)CCSc1ccc(C=O)cc1[N+](=O)[O-]]

To a solution of 4-fluoro-3-nitrobenzaldehyde (8.0 g, 47.31 mmol) in DMF (80 mL) were added 3-mercaptopropionic acid 2-ethylhexyl ester (12.91 mL, 56.77 mmol) and K₂CO₃ (18.34 g, 141.92 mmol) at 25° C. The suspension was stirred at 25° C. for 12 h. The reaction mixture was poured into water (200 mL) and extracted with EtOAc (3×200 mL). The combined organic layer was washed with brine (2×200 mL), dried over Na₂SO₄, filtered and concentrated in vacuo. The residue was purified by silica gel column and concentrated in vacuo to afford 2-ethylhexyl 3-(4-formyl-2-nitro-phenyl)sulfanylpropanoate (17.0 g, Compound 20a) as a yellow oil. ¹H NMR (400 MHz, CDCl₃) δ ppm: 10.03 (s, 1H), 8.72 (d, J=1.6 Hz, 1H), 8.08 (dd, J=8.4 Hz, 1.6 Hz, 1H), 7.60 (d, J=8.4 Hz, 1H), 4.07 (dd, J=5.6 Hz, 2.0 Hz, 2H), 3.34 (t, J=7.6 Hz, 2H), 2.79 (t, J=7.6 Hz, 2H), 1.63-1.59 (m, 1H), 1.34-1.25 (m, 8H), 0.92-0.89 (m, 6H).

### Step 2: Preparation of 2-ethylhexyl 3-[4-[(dimethylamino)methyl]-2-nitro-phenyl]sulfanylpropanoate (Compound 20b)

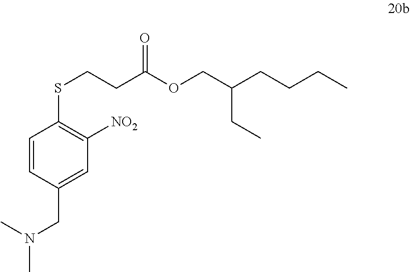 [SMILES: CCCCC(CC)COC(=O)CCSc1ccc(CN(C)C)cc1[N+](=O)[O-]]

Compound 20b was prepared in analogy to Example 1, Step 10 by using 2-ethylhexyl 3-(4-formyl-2-nitro-phenyl)sulfanylpropanoate (Compound 20a) instead of S-(4-formylphenyl) N,N-dimethylcarbamothioate (Compound 1i). 2-Ethylhexyl 3-[4-[(dimethylamino)methyl]-2-nitro-phenyl]sulfanylpropanoate (2.1 g, Compound 20b) was obtained as a light yellow solid. MS obsd. (ESI⁺) [(M+H)⁺]: 397.3.

### Step 3: Preparation of 2-ethylhexyl 3-[2-amino-4-[(dimethylamino)methyl]phenyl]sulfanylpropanoate (Compound 20c)

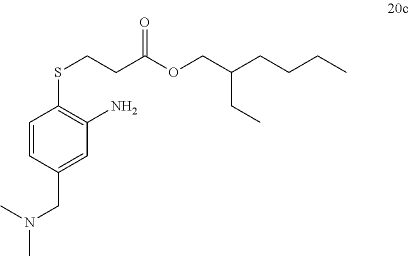 [SMILES: CCCCC(CC)COC(=O)CCSc1ccc(CN(C)C)cc1N]

Compound 20c was prepared in analogy to Example 8, Step 5 by using 2-ethylhexyl 3-[4-[(dimethylamino)methyl]-2-nitro-phenyl]sulfanylpropanoate (Compound 20b) instead of N2,N2,N4-tris[(2,4-dimethoxyphenyl)methyl]-5-iodo-6-methyl-3-nitro-pyridine-2,4-diamine (Compound 8d). 2-Ethylhexyl 3-[2-amino-4-[(dimethylamino)methyl]phenyl]sulfanylpropanoate (2.0 g, Compound 20c) was obtained as a light yellow oil. MS obsd. (ESI⁺) [(M+H)⁺]: 367.3.

### Step 4: Preparation of 7-[2-amino-4-[(dimethylamino)methyl]phenyl]sulfanyl-N,N,1-tris[(2,4-dimethoxyphenyl)methyl]-2-(ethoxymethyl)-6-methyl-imidazo[4,5-c]pyridin-4-amine (Compound 20d)

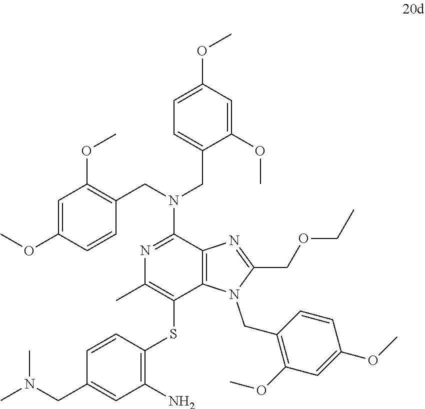

Compound 20d was prepared in analogy to Example 16, Step 9 by using 2-ethylhexyl 3-[2-amino-4-[(dimethylamino)methyl]phenyl]sulfanylpropanoate (Compound 20c) instead of 3-[(dimethylamino)methyl]-2-methyl-6-sulfanyl-phenol (Compound 16h) and N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g). 7-[2-Amino-4-[(dimethylamino)methyl]phenyl]sulfanyl-N,N,1-tris[(2,4-dimethoxyphenyl)methyl]-2-(ethoxymethyl)-6-methyl-imidazo[4,5-c]pyridin-4-amine (400.0 mg, Compound 20d) was obtained as a light yellow oil. MS obsd. (ESI⁺) [(M+H)⁺]: 837.5.

### Step 5: Preparation of 7-[2-amino-4-[(dimethylamino)methyl]phenyl]sulfanyl-2-(ethoxymethyl)-6-methyl-1H-imidazo[4,5-c]pyridin-4-amine (Example 20)

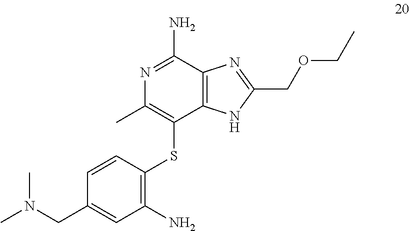 [SMILES: CCOCc1nc2c(N)nc(C)c(Sc3ccc(CN(C)C)cc3N)c2[nH]1]

A solution of 7-[2-amino-4-[(dimethylamino)methyl]phenyl]sulfanyl-N,N,1-tris[(2,4-dimethoxyphenyl)methyl]-2-(ethoxymethyl)-6-methyl-imidazo[4,5-c]pyridin-4-amine (400.0 mg, Compound 20d, 0.48 mmol) in DCM (5.0 mL) and TFA (20.0 mL) was stirred at 20° C. for 2 h. The reaction mixture was concentrated in vacuo. The residue was purified by prep-HPLC and lyophilized to afford 7-[2-amino-4-[(dimethylamino)methyl]phenyl]sulfanyl-2-(ethoxymethyl)-6-methyl-1H-imidazo[4,5-c]pyridin-4-amine (91.63 mg, Example 20) as an off-white solid. MS obsd. (ESI⁺) [(M+H)⁺]: 387.3. ¹H NMR (400 MHz, CD₃OD) δ ppm: 6.77 (d, J=1.6 Hz, 1H), 6.76 (d, J=8.0 Hz, 1H), 6.54 (dd, J=8.0 Hz, 1.6 Hz, 1H), 4.65 (s, 2H), 3.61-3.56 (m, 2H), 3.34 (s, 2H), 2.56 (s, 3H), 2.21 (s, 6H), 1.22 (t, J=7.2 Hz, 3H).

### Example 21

### 2-[(4-amino-2-butyl-6-methyl-1H-imidazo[4,5-c]pyridin-7-yl)sulfanyl]-5-[(dimethylamino)methyl]phenol

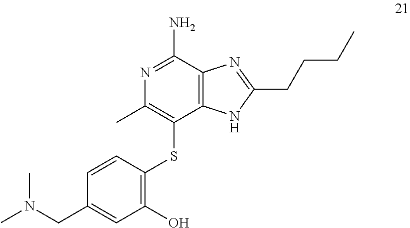 [SMILES: CCCCc1nc2c(N)nc(C)c(Sc3ccc(CN(C)C)cc3O)c2[nH]1]

### Step 1: Preparation of 2-ethylhexyl 3-[4-[(dimethylamino)methyl]-2-hydroxy-phenyl]sulfanylpropanoate (Compound 21a)

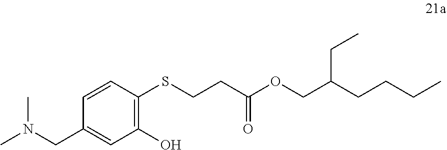 [SMILES: CCCCC(CC)COC(=O)CCSc1ccc(CN(C)C)cc1O]

Compound 21a was prepared in analogy to Example 1, Step 10 by using 2-ethylhexyl 3-(4-formyl-2-hydroxy-phenyl)sulfanylpropanoate (Compound 17a) instead of S-(4-formylphenyl) N,N-dimethylcarbamothioate (Compound 1i). 2-Ethylhexyl 3-[4-[(dimethylamino)methyl]-2-hydroxy-phenyl]sulfanylpropanoate (6.2 g, Compound 21a) was obtained as a light yellow oil. MS obsd. (ESI⁺) [(M+H)⁺]: 368.3.

### Step 2: Preparation of N-[4-amino-2-[bis[(2,4-dimethoxyphenyl)methyl]amino]-5-iodo-6-methyl-3-pyridyl]pentanamide (Compound 21b)

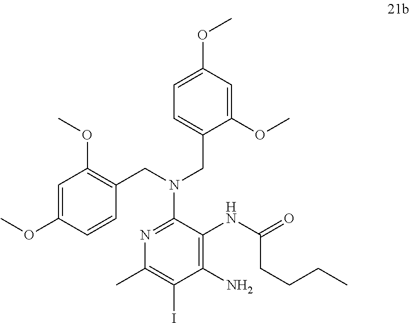 [SMILES: CCCCC(=O)Nc1c(N(Cc2ccc(OC)cc2OC)Cc2ccc(OC)cc2OC)nc(C)c(I)c1N]

Compound 21b was prepared in analogy to Example 1, Step 4 by using N2,N2-bis[(2,4-dimethoxyphenyl)methyl]-5-iodo-6-methyl-pyridine-2,3,4-triamine (Compound 12c) instead of N2,N2-bis[(2,4-dimethoxyphenyl)methyl]-6-methyl-pyridine-2,3,4-triamine (Compound 1c), valeryl chloride instead of 2-ethoxyacetyl chloride. N-[4-amino-2-[bis[(2,4-dimethoxyphenyl)methyl]amino]-5-iodo-6-methyl-3-pyridyl]pentanamide (2.6 g, Compound 21b) was obtained as a light yellow oil, which was used for next step directly without further purification. MS obsd. (ESI⁺) [(M+H)⁺]: 649.3.

### Step 3: Preparation of 2-butyl-N,N-bis[(2,4-dimethoxyphenyl)methyl]-7-iodo-6-methyl-1H-imidazo[4,5-c]pyridin-4-amine (Compound 21c)

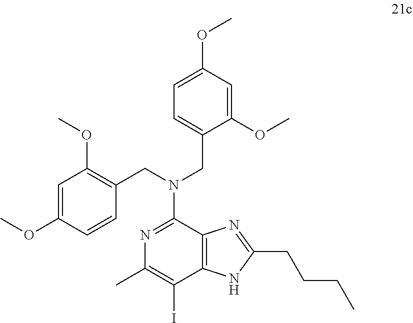 [SMILES: CCCCc1nc2c(N(Cc3ccc(OC)cc3OC)Cc3ccc(OC)cc3OC)nc(C)c(I)c2[nH]1]

Compound 21c was prepared in analogy to Example 1, Step 5 by using N-[4-amino-2-[bis[(2,4-dimethoxyphenyl)methyl]amino]-5-iodo-6-methyl-3-pyridyl]pentanamide (Compound 21b) instead of N-[4-amino-2-[bis[(2,4-dimethoxyphenyl)methyl]amino]-6-methyl-3-pyridyl]-2-ethoxy-acetamide (Compound 1d). 2-Butyl-N,N-bis[(2,4-dimethoxyphenyl)methyl]-7-iodo-6-methyl-1H-imidazo[4,5-c]pyridin-4-amine (2.3 g, Compound 21c) was obtained as a light yellow solid. MS obsd. (ESI⁺) [(M+H)⁺]: 631.2.

### Step 4: Preparation of 2-[[4-[bis[(2,4-dimethoxyphenyl)methyl]amino]-2-butyl-6-methyl-1H-imidazo[4,5-c]pyridin-7-yl]sulfanyl]-5-[(dimethylamino)methyl]phenol (Compound 21d)

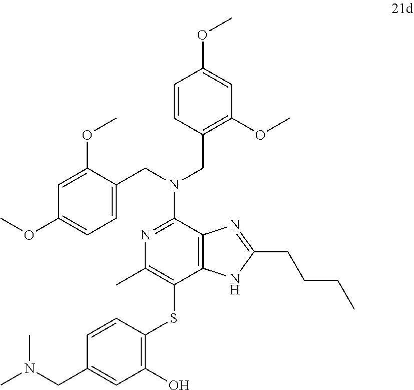 [SMILES: CCCCc1nc2c(N(Cc3ccc(OC)cc3OC)Cc3ccc(OC)cc3OC)nc(C)c(Sc3ccc(CN(C)C)cc3O)c2[nH]1]

Compound 21d was prepared in analogy to Example 16, Step 9 by using 2-butyl-N,N-bis[(2,4-dimethoxyphenyl)methyl]-7-iodo-6-methyl-1H-imidazo[4,5-c]pyridin-4-amine (Compound 21c) instead of 2-(ethoxymethyl)-7-iodo-6-methyl-1H-imidazo[4,5-c]pyridin-4-amine (Compound 1g) and 2-ethylhexyl 3-[4-[(dimethylamino)methyl]-2-hydroxy-phenyl]sulfanylpropanoate (Compound 21a) instead of 3-[(dimethylamino)methyl]-2-methyl-6-sulfanyl-phenol (Compound 16h). 2-[[4-[Bis[(2,4-dimethoxyphenyl)methyl]amino]-2-butyl-6-methyl-1H-imidazo[4,5-c]pyridin-7-yl]sulfanyl]-5-[(dimethylamino)methyl]phenol (200.0 mg, Compound 21d) was obtained as a light yellow solid. MS obsd. (ESI⁺) [(M+H)⁺]: 686.4.

### Step 5: Preparation of 2-[(4-amino-2-butyl-6-methyl-1H-imidazo[4,5-c]pyridin-7-yl)sulfanyl]-5-[(dimethylamino)methyl]phenol (Example 21)

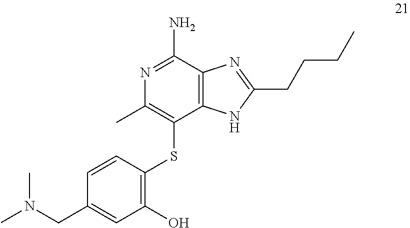 [SMILES: CCCCc1nc2c(N)nc(C)c(Sc3ccc(CN(C)C)cc3O)c2[nH]1]

A solution of 2-[[4-[bis[(2,4-dimethoxyphenyl)methyl]amino]-2-butyl-6-methyl-1H-imidazo[4,5-c]pyridin-7-yl]sulfanyl]-5-[(dimethylamino)methyl]phenol (200.0 mg, Compound 21d, 0.29 mmol) and TFA (2.0 mL) in DCM (2.0 mL) was stirred at 20° C. for 1 h. The reaction mixture was concentrated in vacuo. The residue was purified by prep-HPLC and lyophilized to afford 2-[(4-amino-2-butyl-6-methyl-1H-imidazo[4,5-c]pyridin-7-yl)sulfanyl]-5-[(dimethylamino)methyl]phenol (68.15 mg, Example 21) as a white solid. MS obsd. (ESI⁺) [(M+H)⁺]: 386.2. ¹H NMR (400 MHz, CD₃OD) δ ppm: 6.76 (d, J=1.6 Hz, 1H), 6.57 (dd, J=8.0 Hz, 1.6 Hz, 1H), 6.39 (d, J=8.0 Hz, 1H), 3.33 (s, 2H), 2.82 (t, J=7.6 Hz, 2H), 2.50 (s, 3H), 2.20 (s, 6H), 1.81-1.71 (m, 2H), 1.44-1.32 (m, 2H), 0.94 (t, J=7.2 Hz, 3H).

### Example 22

### 2-[[4-amino-2-(ethoxymethyl)-6-methyl-1H-imidazo[4,5-c]pyridin-7-yl]sulfanyl]-5-(pyrrolidin-1-ylmethyl)phenol

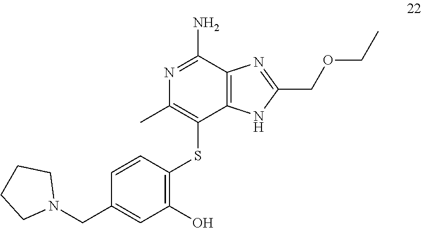 [SMILES: CCOCc1nc2c(N)nc(C)c(Sc3ccc(CN4CCCC4)cc3O)c2[nH]1]

### Step 1: Preparation of 2-ethylhexyl 3-[2-hydroxy-4-(pyrrolidin-1-ylmethyl)phenyl]sulfanylpropanoate (Compound 22a)

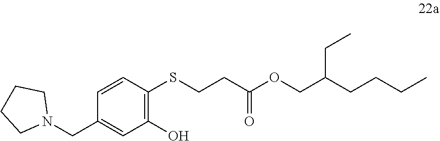 [SMILES: CCCCC(CC)COC(=O)CCSc1ccc(CN2CCCC2)cc1O]

Compound 22a was prepared in analogy to Example 17, Step 2 by using pyrrolidine instead of diethylamine. 2-Ethylhexyl 3-[2-hydroxy-4-(pyrrolidin-1-ylmethyl)phenyl]sulfanylpropanoate (568.0 mg, Compound 22a) was obtained as a yellow oil. MS obsd. (ESI⁺) [(M+H)⁺]: 394.3. ¹H NMR (400 MHz, CDCl₃) δ ppm: 7.40 (dd, J=8.0 Hz, 1.6 Hz, 1H), 7.09 (d, J=1.6 Hz, 1H), 6.91 (d, J=8.0 Hz, 1H), 4.08-3.99 (m, 2H), 3.90 (s, 2H), 3.00 (t, J=7.2 Hz, 2H), 2.97-2.91 (m, 4H), 2.57 (t, J=7.2 Hz, 2H), 2.01-1.91 (m, 4H), 1.62-1.53 (m, 1H), 1.43-1.34 (m, 2H), 1.34-1.25 (m, 6H), 0.90 (t, J=7.2 Hz, 6H).

### Step 2: Preparation of 5-(pyrrolidin-1-ylmethyl)-2-sulfanyl-phenol (Compound 22b)

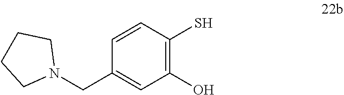 [SMILES: Oc1cc(CN2CCCC2)ccc1S]

To a solution of 2-ethylhexyl 3-[2-hydroxy-4-(pyrrolidin-1-ylmethyl)phenyl]sulfanylpropanoate (557.0 mg, Compound 22a, 1.42 mmol) in THF (10 mL) was added t-BuOK (4.25 mL, 4.25 mmol, 1.0 M in THF) dropwise at −78° C. The mixture was stirred at −78° C. for 1 h. Then the pH of the mixture was adjusted to 5 with FA at −78° C. and the mixture was concentrated in vacuo. The residue was purified by reversed prep-HPLC and lyophilized to afford 5-(pyrrolidin-1-ylmethyl)-2-sulfanyl-phenol (200.0 mg, Compound 22b) as an off-white solid. MS obsd. (ESI⁺) [(M+H)⁺]: 210.2.

### Step 3: Preparation of 2-[[4-amino-2-(ethoxymethyl)-6-methyl-1H-imidazo[4,5-c]pyridin-7-yl]sulfanyl]-5-(pyrrolidin-1-ylmethyl)phenol (Example 22)

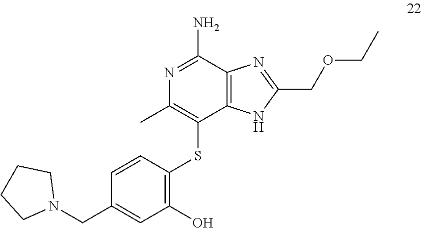 [SMILES: CCOCc1nc2c(N)nc(C)c(Sc3ccc(CN4CCCC4)cc3O)c2[nH]1]

The title compound was prepared in analogy to Example 16, Step 9 by using 5-(pyrrolidin-1-ylmethyl)-2-sulfanyl-phenol (Compound 22b) instead of 3-[(dimethylamino)methyl]-2-methyl-6-sulfanyl-phenol (Compound 16h). 2-[[4-Amino-2-(ethoxymethyl)-6-methyl-1H-imidazo[4,5-c]pyridin-7-yl]sulfanyl]-5-(pyrrolidin-1-ylmethyl)phenol (32.1 mg, Example 22) was obtained as a white solid. MS obsd. (ESI⁺) [(M+H)⁺]: 414.3. ¹H NMR (400 MHz, DMSO-d₆) δ ppm: 12.45 (br s, 1H), 9.90 (br s, 1H), 6.80 (s, 1H), 6.61-6.39 (m, 3H), 6.10-6.30 (m, 1H), 4.51 (s, 2H), 3.56-3.43 (m, 2H), 3.34 (s, 2H), 2.44-2.31 (m, 7H), 1.70-1.60 (m, 4H), 1.11 (t, J=7.2 Hz, 3H).

### Example 23

### 2-[[4-amino-2-(ethoxymethyl)-6-methyl-1H-imidazo[4,5-c]pyridin-7-yl]sulfanyl]-5-[(dimethylamino)methyl]-4-methyl-phenol

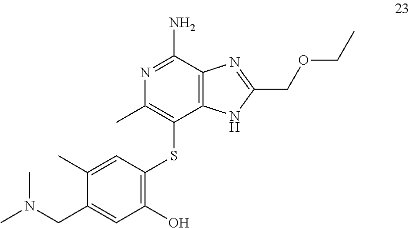 [SMILES: CCOCc1nc2c(N)nc(C)c(Sc3cc(C)c(CN(C)C)cc3O)c2[nH]1]

Step 1: Preparation of 4-bromo-5-hydroxy-2-methyl-benzaldehyde (Compound 23a)

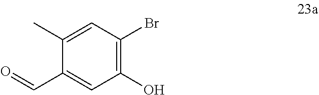

To a solution of 5-hydroxy-2-methyl-benzaldehyde (450.0 mg, 3.31 mmol) in methanol (10 mL) was added pyridinium hydrobromide perbromide (1.05 g, 3.31 mmol) at 25° C. The mixture was stirred at 25° C. for 1 h. The mixture was concentrated in vacuo and the residue was purified by reversed prep-HPLC to afford 4-bromo-5-hydroxy-2-methyl-benzaldehyde (500.0 mg, Compound 23a) as a white solid. MS obsd. (ESI⁺) [(M+H)⁺]: 215.2.

### Step 2: Preparation of 2-ethylhexyl 3-(4-formyl-2-hydroxy-5-methyl-phenyl)sulfanylpropanoate (Compound 23b)

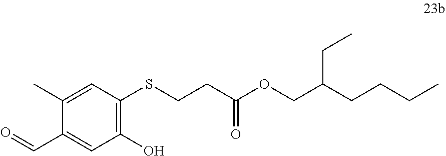 [SMILES: CCCCC(CC)COC(=O)CCSc1cc(C)c(C=O)cc1O]

Compound 23b was prepared in analogy to Example 17, Step 1 by using 4-bromo-5-hydroxy-2-methyl-benzaldehyde (Compound 23a) instead of 3-hydroxy-4-iodo-benzaldehyde. 2-Ethylhexyl 3-(4-formyl-2-hydroxy-5-methyl-phenyl)sulfanylpropanoate (480.0 mg, Compound 23b) was obtained as a yellow oil. MS obsd. (ESI⁺) [(M+H)⁺]: 353.2.

### Step 3: Preparation of 2-ethylhexyl 3-[4-[(dimethylamino)methyl]-2-hydroxy-5-methyl-phenyl]sulfanylpropanoate (Compound 23c)

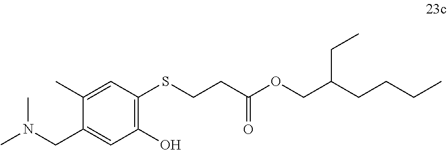 [SMILES: CCCCC(CC)COC(=O)CCSc1cc(C)c(CN(C)C)cc1O]

Compound 23c was prepared in analogy to Example 17, Step 2 by using 2-ethylhexyl 3-(4-formyl-2-hydroxy-5-methyl-phenyl)sulfanylpropanoate (Compound 23b) instead of 2-ethylhexyl 3-(4-formyl-2-hydroxy-phenyl)sulfanylpropanoate (Compound 17a), dimethylamine hydrochloride instead of diethylamine. 2-Ethylhexyl 3-[4-[(dimethylamino)methyl]-2-hydroxy-5-methyl-phenyl]sulfanylpropanoate (380.0 mg, Compound 23c) was obtained as an orange oil. MS obsd. (ESI⁺) [(M+H)⁺]: 382.3.

### Step 4: Preparation of 2-[[4-amino-2-(ethoxymethyl)-6-methyl-1H-imidazo[4,5-c]pyridin-7-yl]sulfanyl]-5-[(dimethylamino)methyl]-4-methyl-phenol (Example 23)

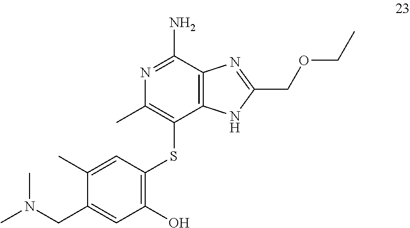 [SMILES: CCOCc1nc2c(N)nc(C)c(Sc3cc(C)c(CN(C)C)cc3O)c2[nH]1]

The title compound was prepared in analogy to Example 16, Step 9 by using 2-ethylhexyl 3-[4-[(dimethylamino)methyl]-2-hydroxy-5-methyl-phenyl]sulfanylpropanoate (Compound 23c) instead of 3-[(dimethylamino)methyl]-2-methyl-6-sulfanyl-phenol (Compound 16h). 2-[[4-Amino-2-(ethoxymethyl)-6-methyl-1H-imidazo[4,5-c]pyridin-7-yl]sulfanyl]-5-[(dimethylamino)methyl]-4-methyl-phenol (17.3 mg, Example 23) was obtained as a white solid. MS obsd. (ESI⁺) [(M+H)⁺]: 402.2. ¹H NMR (400 MHz, CD₃OD) δ ppm: 6.83 (s, 1H), 6.41 (s, 1H), 4.64 (s, 2H), 3.98 (s, 2H), 3.63-3.57 (m, 2H), 2.69 (s, 6H), 2.53 (s, 3H), 2.09 (s, 3H), 1.21 (t, J=7.2 Hz, 3H).

### Example 24

### 2-[[4-amino-2-(ethoxymethyl)-6-methyl-1H-imidazo[4,5-c]pyridin-7-yl]sulfanyl]-5-[1-(dimethylamino)ethyl]phenol

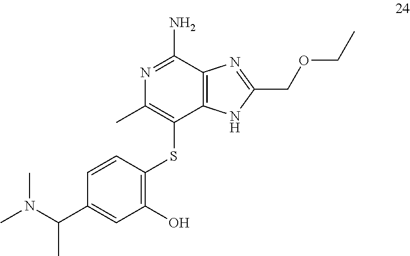 [SMILES: CCOCc1nc2c(N)nc(C)c(Sc3ccc(C(C)N(C)C)cc3O)c2[nH]1]

### Step 1: Preparation of 1-(3-hydroxy-4-iodo-phenyl)ethanone (Compound 24a)

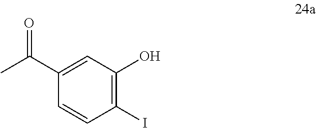

To a solution of 1-(3-hydroxyphenyl)ethanone (5.0 g, 36.72 mmol) in acetic acid (50 mL) was added NIS (9.09 g, 40.4 mmol) at 25° C. The mixture was stirred at 25° C. for 1 h, then diluted with H₂O (60 mL). The suspension was stirred at 25° C. for 0.5 h, then filtered and the collected solid was washed with H₂O (3×30 mL). The solid was dried in vacuo and the residue was purified by silica gel column to afford 1-(3-hydroxy-4-iodo-phenyl)ethanone (3.89 g, Compound 24a) as a white solid. MS obsd. (ESI⁺) [(M+H)⁺]: 262.9. ¹H NMR (400 MHz, CDCl₃) δ ppm: 7.80 (d, J=8.0 Hz, 1H), 7.57 (d, J=2.0 Hz, 1H), 7.26 (dd, J=8.0 Hz, 2.0 Hz, 1H), 5.83 (br s, 1H), 2.58 (s, 3H).

### Step 2: Preparation of 2-ethylhexyl 3-(4-acetyl-2-hydroxy-phenyl)sulfanylpropanoate (Compound 24b)

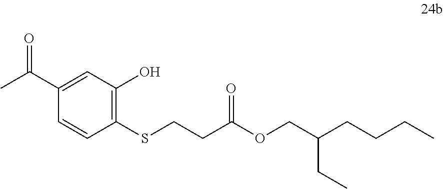 [SMILES: CCCCC(CC)COC(=O)CCSc1ccc(C(C)=O)cc1O]

Compound 24b was prepared in analogy to Example 17, Step 1 by using 1-(3-hydroxy-4-iodo-phenyl)ethanone (Compound 24a) instead of 3-hydroxy-4-iodo-benzaldehyde. 2-Ethylhexyl 3-(4-acetyl-2-hydroxy-phenyl)sulfanylpropanoate (1.73 g, Compound 24b) was obtained as a yellow solid. MS obsd. (ESI⁺) [(M+H)⁺]: 353.3. ¹H NMR (400 MHz, CDCl₃) δ ppm: 7.56 (d, J=8.0 Hz, 2H), 7.55 (d, J=2.0 Hz, 1H), 7.47 (dd, J=8.0 Hz, 2.0 Hz, 1H), 6.99 (br s, 1H), 4.09-4.01 (m, 2H), 3.03 (t, J=6.8 Hz, 2H), 2.59 (s, 3H), 2.58-2.54 (m, 2H), 1.69-1.48 (m, 1H), 1.40-1.34 (m, 2H), 1.34-1.27 (m, 6H), 0.90 (t, J=7.6 Hz, 6H).

### Step 3: Preparation of 1-[4-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-3-hydroxy-phenyl]ethanone (Compound 24c)

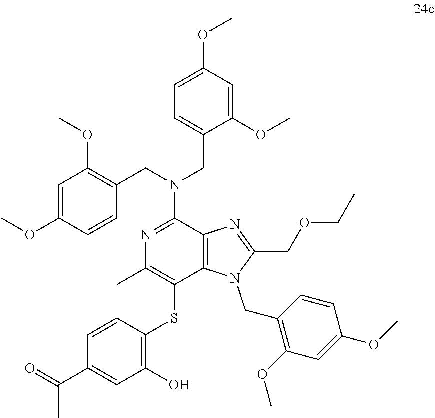

Compound 24c was prepared in analogy to Example 16, Step 9 by using N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g) and 2-ethylhexyl 3-(4-acetyl-2-hydroxy-phenyl)sulfanylpropanoate (Compound 24b) instead of 3-[(dimethylamino)methyl]-2-methyl-6-sulfanyl-phenol (Compound 16h). 1-[4-[4-[Bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-3-hydroxy-phenyl]ethanone (350.0 mg, Compound 24c) was obtained as a light yellow solid. MS obsd. (ESI⁺) [(M+H)⁺]: 823.4.

### Step 4: Preparation of 5-(1-aminoethyl)-2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-phenol (Compound 24d)

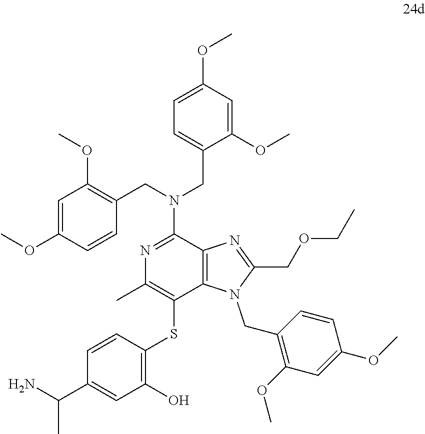

To a solution of 1-[4-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-3-hydroxy-phenyl]ethanone (1.03 g, Compound 24c, 1.26 mmol) in methanol (15 mL) were added NH₄OAc (1.45 g, 18.88 mmol) and NaBH₃CN (791.07 mg, 12.59 mmol) at 25° C. The suspension was heated to 70° C. and stirred at 70° C. for 12 h, then filtered through celite. The solid was washed with MeOH (20 mL). The filtrate was concentrate in vacuo. The residue was purified by reversed prep-HPLC and lyophilized. The residue was suspended in DCM/MeOH (V/V=10/1, 50 mL) and stirred at 20° C. for 1 h. The suspension was filtered and the filtrate was concentrated in vacuo to afford 5-(1-aminoethyl)-2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-phenol (400.0 mg, Compound 24d) as a light brown solid. MS obsd. (ESI⁺) [(M+H)⁺]: 824.6.

### Step 5: Preparation of 2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-5-[1-(dimethylamino)ethyl]phenol (Compound 24e)

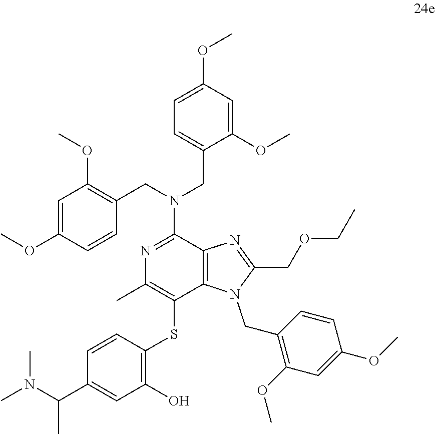

To a solution of 5-(1-aminoethyl)-2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-phenol (400.0 mg, Compound 24d, 0.49 mmol) in DCM (5.0 mL) were added formaldehyde (197.0 mg, 2.43 mmol) and NaBH(OAc)₃ (514.42 mg, 2.43 mmol) at 20° C. The suspension was stirred at 20° C. for 2 h, and then filtered through celite. The collected solid was washed with MeOH (50 mL). The filtrate was concentrated in vacuo and the residue was purified by reversed prep-HPLC and lyophilized to afford 2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-5-[1-(dimethylamino)ethyl]phenol (212.0 mg, Compound 24e) as a colorless oil. MS obsd. (ESI⁺) [(M+H)⁺]: 852.5.

### Step 6: Preparation of 2-[[4-amino-2-(ethoxymethyl)-6-methyl-1H-imidazo[4,5-c]pyridin-7-yl]sulfanyl]-5-[1-(dimethylamino)ethyl]phenol (Example 24)

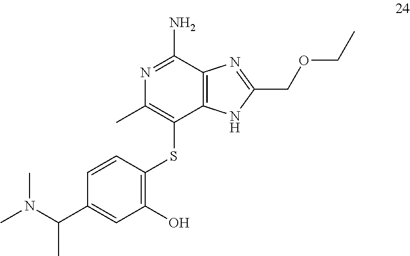 [SMILES: CCOCc1nc2c(N)nc(C)c(Sc3ccc(C(C)N(C)C)cc3O)c2[nH]1]

A solution of 2-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-5-[1-(dimethylamino)ethyl]phenol (212.0 mg, Compound 24e, 0.25 mmol) in TFA (6.0 mL) was stirred at 25° C. for 16 h. The mixture was concentrated in vacuo. The residue was purified by prep-HPLC and lyophilized to afford 2-[[4-amino-2-(ethoxymethyl)-6-methyl-1H-imidazo[4,5-c]pyridin-7-yl]sulfanyl]-5-[1-(dimethylamino)ethyl]phenol (33.19 mg Example 24) as a white solid. MS obsd. (ESI⁺) [(M+H)⁺]: 402.3. ¹H NMR (400 MHz, CD₃OD) δ ppm: 6.76 (d, J=1.6 Hz, 1H), 6.58 (dd, J=8.0 Hz, 1.6 Hz, 1H), 6.44 (d, J=8.0 Hz, 1H), 4.64 (s, 2H), 3.62-3.55 (m, 2H), 3.25-3.19 (m, 1H), 2.54 (s, 3H), 2.18 (s, 6H), 1.34 (d, J=6.8 Hz, 3H), 1.21 (t, J=7.2 Hz, 3H).

### Example 25

### 5-[[4-amino-2-(ethoxymethyl)-6-methyl-1H-imidazo[4,5-c]pyridin-7-yl]sulfanyl]-2-[(dimethylamino)methyl]-4-methyl-phenol

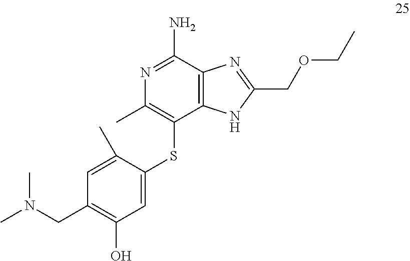 [SMILES: CCOCc1nc2c(N)nc(C)c(Sc3cc(O)c(CN(C)C)cc3C)c2[nH]1]

### Step 1: Preparation of 2-ethylhexyl 3-(4-formyl-5-hydroxy-2-methyl-phenyl)sulfanylpropanoate (Compound 25a)

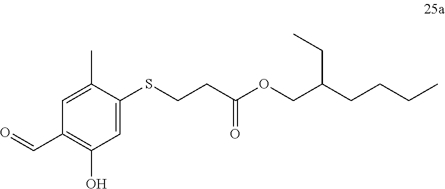 [SMILES: CCCCC(CC)COC(=O)CCSc1cc(O)c(C=O)cc1C]

Compound 25a was prepared in analogy to Example 17, Step 1 by using 4-bromo-2-hydroxy-5-methyl-benzaldehyde instead of 3-hydroxy-4-iodo-benzaldehyde. 2-Ethylhexyl 3-(4-formyl-5-hydroxy-2-methyl-phenyl)sulfanylpropanoate (600.0 mg, Compound 25a) was obtained as a yellow oil.

### Step 2: Preparation of 2-ethylhexyl 3-[4-[(dimethylamino)methyl]-5-hydroxy-2-methyl-phenyl]sulfanylpropanoate (Compound 25b)

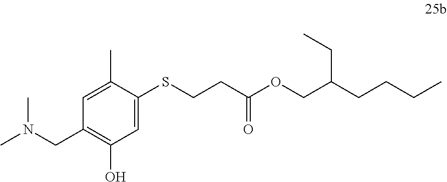 [SMILES: CCCCC(CC)COC(=O)CCSc1cc(O)c(CN(C)C)cc1C]

Compound 25b was prepared in analogy to Example 9, Step 5 by using 2-ethylhexyl 3-(4-formyl-5-hydroxy-2-methyl-phenyl)sulfanylpropanoate (Compound 25a) instead of 4-[[4-amino-2-(ethoxymethyl)-6-methyl-1H-imidazo[4,5-c]pyridin-7-yl]sulfanyl]-2-methoxy-benzaldehyde (Compound 9d). 2-Ethylhexyl 3-[4-[(dimethylamino)methyl]-5-hydroxy-2-methyl-phenyl]sulfanylpropanoate (100.0 mg Compound 25b) was obtained as a yellow oil. MS obsd. (ESI⁺) [(M+H)⁺]: 382.4.

### Step 3: Preparation of 5-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-2-[(dimethylamino)methyl]-4-methyl-phenol (Compound 25c)

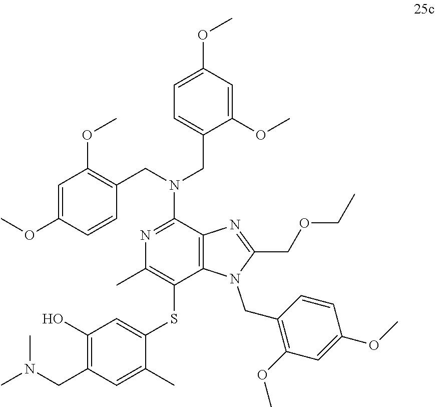

Compound 25c was prepared in analogy to Example 16, Step 9 by using N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g) and 2-ethylhexyl 3-[4-[(dimethylamino)methyl]-5-hydroxy-2-methyl-phenyl]sulfanylpropanoate (Compound 25b) instead of 3-[(dimethylamino)methyl]-2-methyl-6-sulfanyl-phenol (Compound 16h). 5-[4-[Bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-2-[(dimethylamino)methyl]-4-methyl-phenol (50.0 mg, Compound 25c) was obtained as a brown solid. MS obsd. (ESI⁺) [(M+H)⁺]: 852.4.

### Step 4: Preparation of 5-[[4-amino-2-(ethoxymethyl)-6-methyl-1H-imidazo[4,5-c]pyridin-7-yl]sulfanyl]-2-[(dimethylamino)methyl]-4-methyl-phenol (Example 25)

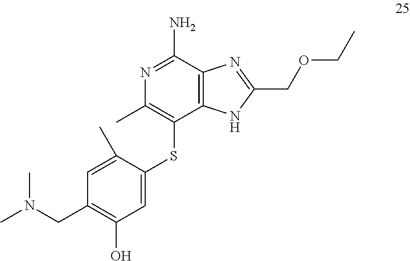 [SMILES: CCOCc1nc2c(N)nc(C)c(Sc3cc(O)c(CN(C)C)cc3C)c2[nH]1]

A solution of 5-[4-[bis[(2,4-dimethoxyphenyl)methyl]amino]-1-[(2,4-dimethoxyphenyl)methyl]-2-(ethoxymethyl)-6-methyl-imidazo[4,5-c]pyridin-7-yl]sulfanyl-2-[(dimethylamino)methyl]-4-methyl-phenol (45.0 mg, Compound 25c) in TFA (2.0 mL) was stirred at 25° C. for 2 h. The mixture was concentrated in vacuo. The residue was purified by prep-HPLC and lyophilized to afford 5-[[4-amino-2-(ethoxymethyl)-6-methyl-1H-imidazo[4,5-c]pyridin-7-yl]sulfanyl]-2-[(dimethylamino)methyl]-4-methyl-phenol (11.0 mg, Example 25) as a white solid. MS obsd. (ESI⁺) [(M+H)⁺]: 402.1. ¹H NMR (400 MHz, CD₃OD) δ ppm: 6.98 (s, 1H), 5.96 (s, 1H), 4.63 (s, 2H), 3.83 (s, 2H), 3.63-3.55 (m, 2H), 2.51 (s, 6H), 2.50 (s, 3H), 2.37 (s, 3H), 1.21 (t, J=7.2 Hz, 3H).

### Example 26

### 7-[2-amino-4-(pyrrolidin-1-ylmethyl)phenyl]sulfanyl-2-(ethoxymethyl)-6-methyl-1H-imidazo[4,5-c]pyridin-4-amine

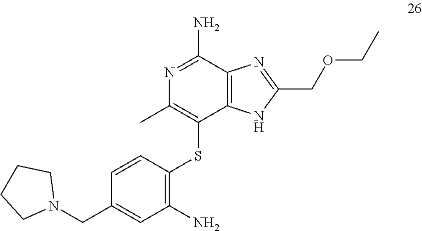 [SMILES: CCOCc1nc2c(N)nc(C)c(Sc3ccc(CN4CCCC4)cc3N)c2[nH]1]

### Step 1: Preparation of 2-ethylhexyl 3-[2-nitro-4-(pyrrolidin-1-ylmethyl)phenyl]sulfanylpropanoate (Compound 26a)

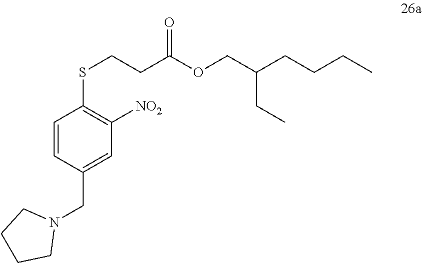 [SMILES: CCCCC(CC)COC(=O)CCSc1ccc(CN2CCCC2)cc1[N+](=O)[O-]]

Compound 26a was prepared in analogy to Example 20, Step 2 by using pyrrolidine instead of dimethylamine hydrochloride. 2-Ethylhexyl 3-[2-nitro-4-(pyrrolidin-1-ylmethyl)phenyl]sulfanylpropanoate (1000.0 mg, Compound 26a) was obtained as a light yellow oil. MS obsd. (ESI⁺) [(M+H)⁺]: 423.3.

### Step 2: Preparation of 2-ethylhexyl 3-[2-amino-4-(pyrrolidin-1-ylmethyl)phenyl]sulfanylpropanoate (Compound 26b)

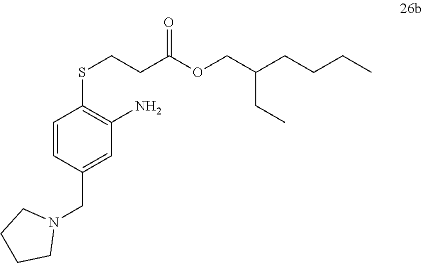 [SMILES: CCCCC(CC)COC(=O)CCSc1ccc(CN2CCCC2)cc1N]

Compound 26b was prepared in analogy to Example 20, Step 3 by using 2-ethylhexyl 3-[2-nitro-4-(pyrrolidin-1-ylmethyl)phenyl]sulfanylpropanoate (Compound 26a) instead of 2-ethylhexyl 3-[4-[(dimethylamino)methyl]-2-nitro-phenyl]sulfanylpropanoate (Compound 20b). 2-Ethylhexyl 3-[2-amino-4-(pyrrolidin-1-ylmethyl)phenyl]sulfanylpropanoate (2.0 g, Compound 26b) was obtained as a light yellow oil. MS obsd. (ESI⁺) [(M+H)⁺]: 393.3.

### Step 3: Preparation of 7-[2-amino-4-(pyrrolidin-1-ylmethyl)phenyl]sulfanyl-N,N,1-tris[(2,4-dimethoxyphenyl)methyl]-2-(ethoxymethyl)-6-methyl-imidazo[4,5-c]pyridin-4-amine (Compound 26c)

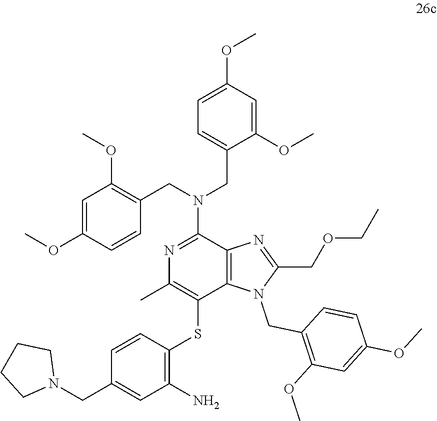

Compound 26c was prepared in analogy to Example 16, Step 9 by using 2-ethylhexyl 3-[2-amino-4-[(dimethylamino)methyl]phenyl]sulfanylpropanoate (Compound 26b) instead of 3-[(dimethylamino)methyl]-2-methyl-6-sulfanyl-phenol (Compound 16h) and N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g). 7-[2-Amino-4-(pyrrolidin-1-ylmethyl)phenyl]sulfanyl-N,N,1-tris[(2,4-dimethoxyphenyl)methyl]-2-(ethoxymethyl)-6-methyl-imidazo[4,5-c]pyridin-4-amine (180.0 mg, Compound 26c) was obtained as a light yellow oil. MS obsd. (ESI⁺) [(M+H)⁺]: 863.5.

### Step 4: Preparation of 7-[2-amino-4-(pyrrolidin-1-ylmethyl)phenyl]sulfanyl-2-(ethoxymethyl)-6-methyl-LH-imidazo[4,5-c]pyridin-4-amine (Example 26)

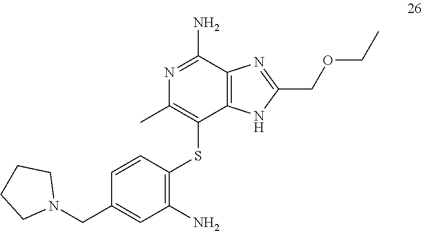 [SMILES: CCOCc1nc2c(N)nc(C)c(Sc3ccc(CN4CCCC4)cc3N)c2[nH]1]

A solution of 7-[2-amino-4-(pyrrolidin-1-ylmethyl)phenyl]sulfanyl-N,N,1-tris[(2,4-dimethoxyphenyl)methyl]-2-(ethoxymethyl)-6-methyl-imidazo[4,5-c]pyridin-4-amine (180.0 mg, Compound 26c, 0.21 mmol) in DCM (2.0 mL) and TFA (10.0 mL) was stirred at 20° C. for 12 h.

The reaction mixture was concentrated in vacuo. The crude product was further purified by prep-HPLC and lyophilized to afford 7-[2-amino-4-(pyrrolidin-1-ylmethyl)phenyl]sulfanyl-2-(ethoxymethyl)-6-methyl-1H-imidazo[4,5-c]pyridin-4-amine (25.82 mg, Example 26) as a white solid. MS obsd. (ESI⁺) [(M+H)⁺]: 413.3. ¹H NMR (400 MHz, CD₃OD) δ ppm: 6.81 (d, J=1.6 Hz, 1H), 6.77 (d, J=8.0 Hz, 1H), 6.57 (dd, J=8.0 Hz, 1.6 Hz, 1H), 4.65 (s, 2H), 3.62-3.56 (m, 2H), 3.54 (s, 2H), 2.58-2.57 (m, 4H), 2.56 (s, 3H), 1.81-1.79 (m, 4H), 1.22 (t, J=7.2 Hz, 3H).

### Example 27

### 7-[2-amino-4-[(4-methylpiperazin-1-yl)methyl]phenyl]sulfanyl-2-(ethoxymethyl)-6-methyl-1H-imidazo[4,5-c]pyridin-4-amine

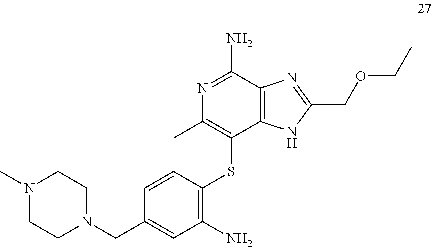 [SMILES: CCOCc1nc2c(N)nc(C)c(Sc3ccc(CN4CCN(C)CC4)cc3N)c2[nH]1]

### Step 1: Preparation of 2-ethylhexyl 3-[4-[(4-methylpiperazin-1-yl)methyl]-2-nitro-phenyl]sulfanylpropanoate (Compound 27a)

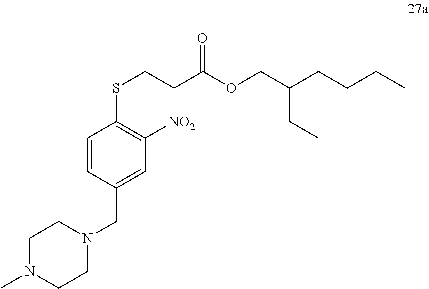 [SMILES: CCCCC(CC)COC(=O)CCSc1ccc(CN2CCN(C)CC2)cc1[N+](=O)[O-]]

Compound 27a was prepared in analogy to Example 20, Step 2 by using methylpiperazine instead of dimethylamine hydrochloride. 2-Ethylhexyl 3-[4-[(4-methylpiperazin-1-yl)methyl]-2-nitro-phenyl]sulfanylpropanoate (900.0 mg, Compound 27a) was obtained as a light yellow oil. MS obsd. (ESI⁺) [(M+H)⁺]: 452.3.

### Step 2: Preparation of 2-ethylhexyl 3-[2-amino-4-[(4-methylpiperazin-1-yl)methyl]phenyl]sulfanylpropanoate (Compound 27b)

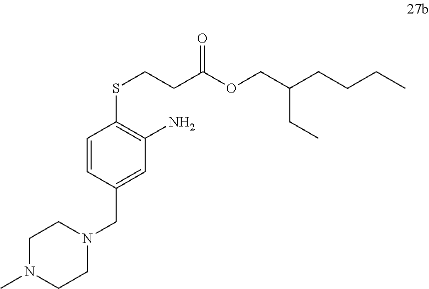 [SMILES: CCCCC(CC)COC(=O)CCSc1ccc(CN2CCN(C)CC2)cc1N]

Compound 27b was prepared in analogy to Example 20, Step 3 by using 2-ethylhexyl 3-[4-[(4-methylpiperazin-1-yl)methyl]-2-nitro-phenyl]sulfanylpropanoate (Compound 27a) instead of 2-ethylhexyl 3-[4-[(dimethylamino)methyl]-2-nitro-phenyl]sulfanylpropanoate (Compound 20b). 2-Ethylhexyl 3-[2-amino-4-[(4-methylpiperazin-1-yl)methyl]phenyl]sulfanylpropanoate (2.0 g, Compound 27b) was obtained as light brown oil. MS obsd. (ESI) [(M+H)⁺]: 422.6.

### Step 3: Preparation of 7-[2-amino-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (Compound 27c)

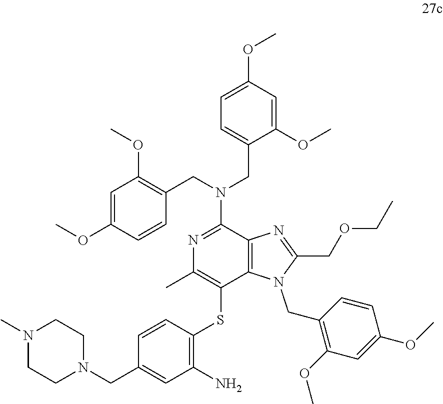

Compound 27c was prepared in analogy to Example 16, Step 9 by using 2-ethylhexyl 3-[2-amino-4-[(4-methylpiperazin-1-yl)methyl]phenyl]sulfanylpropanoate (Compound 27b) instead of 3-[(dimethylamino)methyl]-2-methyl-6-sulfanyl-phenol (Compound 16h), N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g). 7-[2-Amino-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (200.0 mg, Compound 27c) was obtained as a light yellow oil. MS obsd. (ESI⁺) [(M+H)].: 892.5.

### Step 4: Preparation of 7-[2-amino-4-[(4-methylpiperazin-1-yl)methyl]phenyl]sulfanyl-2-(ethoxymethyl)-6-methyl-LH-imidazo[4,5-c]pyridin-4-amine (Example 27)

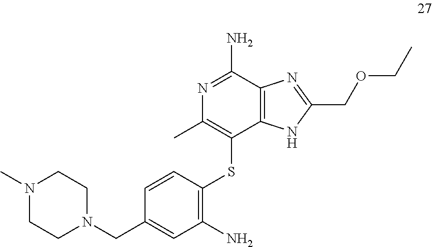 [SMILES: CCOCc1nc2c(N)nc(C)c(Sc3ccc(CN4CCN(C)CC4)cc3N)c2[nH]1]

The title compound was prepared in analogy to Example 20, Step 5 by using 7-[2-amino-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (Compound 27c) instead of 7-[2-amino-4-[(dimethylamino)methyl]phenyl]sulfanyl-N,N,1-tris[(2,4-dimethoxyphenyl)methyl]-2-(ethoxymethyl)-6-methyl-imidazo[4,5-c]pyridin-4-amine (Compound 20d). 7-[2-Amino-4-[(4-methylpiperazin-1-yl)methyl]phenyl]sulfanyl-2-(ethoxymethyl)-6-methyl-1H-imidazo[4,5-c]pyridin-4-amine (31.92 mg, Example 27) was obtained as a white solid. MS obsd. (ESI⁺) [(M+H)⁺]: 442.3. ¹H NMR (400 MHz, CD₃OD) δ ppm: 6.80 (d, J=1.6 Hz, 1H), 6.78 (d, J=8.0 Hz, 1H), 6.56 (dd, J=8.0 Hz, 1.6 Hz, 1H), 4.65 (s, 2H), 3.62-3.56 (m, 2H), 3.39 (s, 2H), 2.57 (s, 3H), 2.56-2.41 (m, 8H), 2.31 (s, 3H), 1.23 (t, J=7.2 Hz, 3H).

### Example 28

### 7-[3-amino-4-[(4-methylpiperazin-1-yl)methyl]phenyl]sulfanyl-2-(ethoxymethyl)-6-methyl-1H-imidazo[4,5-c]pyridin-4-amine

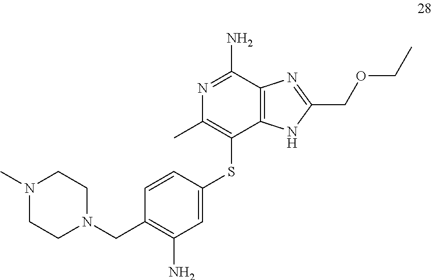 [SMILES: CCOCc1nc2c(N)nc(C)c(Sc3ccc(CN4CCN(C)CC4)c(N)c3)c2[nH]1]

### Step 1: Preparation of 2-ethylhexyl 3-(4-formyl-3-nitro-phenyl)sulfanylpropanoate (Compound 28a)

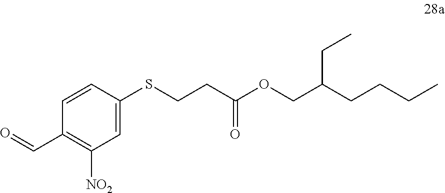 [SMILES: CCCCC(CC)COC(=O)CCSc1ccc(C=O)c([N+](=O)[O-])c1]

Compound 28a was prepared in analogy to Example 17, Step 1 by using 4-bromo-2-nitrobenzaldehyde instead of 3-hydroxy-4-iodo-benzaldehyde. 2-Ethylhexyl 3-(4-formyl-3-nitro-phenyl)sulfanylpropanoate (2.40 g, Compound 28a) was obtained as a yellow oil. ¹H NMR (400 MHz, DMSO-d₆) δ ppm: 10.09 (s, 1H), 7.95 (d, J=1.6 Hz, 1H), 7.88 (d, J=8.0 Hz, 1H), 7.77 (dd, J=8.0 Hz, 1.6 Hz, 1H), 3.96 (d, J=5.6 Hz, 2H), 3.39 (t, J=6.8 Hz, 2H), 2.74 (t, J=6.8 Hz, 2H), 1.57-1.47 (m, 1H), 1.34-1.26 (m, 2H), 1.25-1.20 (m, 6H), 0.91-0.82 (m, 6H).

### Step 2: Preparation of 2-ethylhexyl 3-[4-[(4-methylpiperazin-1-yl)methyl]-3-nitro-phenyl]sulfanylpropanoate (Compound 28b)

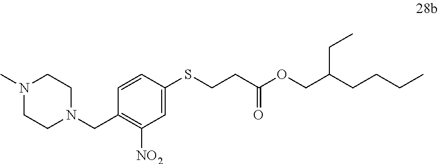 [SMILES: CCCCC(CC)COC(=O)CCSc1ccc(CN2CCN(C)CC2)c([N+](=O)[O-])c1]

Compound 28b was prepared in analogy to Example 17, Step 2 by using 2-ethylhexyl 3-(4-formyl-3-nitro-phenyl)sulfanylpropanoate (Compound 28a) instead of 2-ethylhexyl 3-(4-formyl-2-hydroxy-phenyl)sulfanylpropanoate (Compound 17a), 1-methylpiperazine instead of diethylamine. 2-Ethylhexyl 3-[4-[(4-methylpiperazin-1-yl)methyl]-3-nitro-phenyl]sulfanylpropanoate (290.0 mg, Compound 28b) was obtained as an orange oil. MS obsd. (ESI⁺) [(M+H)⁺]: 452.3.

### Step 3: Preparation of 2-ethylhexyl 3-[3-amino-4-[(4-methylpiperazin-1-yl)methyl]phenyl]sulfanylpropanoate (Compound 28c)

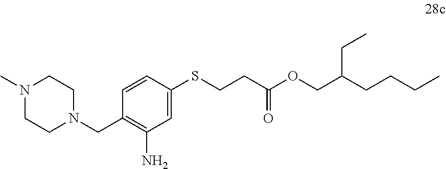 [SMILES: CCCCC(CC)COC(=O)CCSc1ccc(CN2CCN(C)CC2)c(N)c1]

Compound 28c was prepared in analogy to Example 20, Step 3 by using 2-ethylhexyl 3-[4-[(4-methylpiperazin-1-yl)methyl]-3-nitro-phenyl]sulfanylpropanoate (Compound 28b) instead of 2-ethylhexyl 3-[4-[(dimethylamino)methyl]-2-nitro-phenyl]sulfanylpropanoate (Compound 20b). 2-Ethylhexyl 3-[3-amino-4-[(4-methylpiperazin-1-yl)methyl]phenyl]sulfanylpropanoate (220.0 mg, Compound 28c) was obtained as a grey solid. MS obsd. (EST⁺) [(M+H)⁺]: 422.2 [(M+H)⁺].

### Step 4: Preparation of 7-[3-amino-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (Compound 28d)

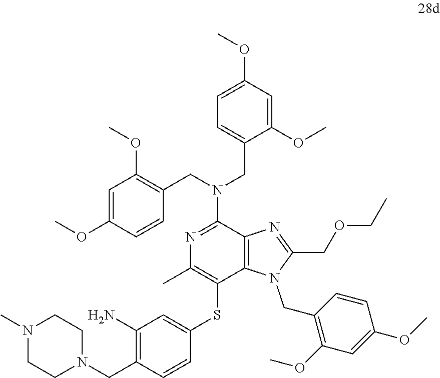

Compound 28d was prepared in analogy to Example 16, Step 9 by 2-ethylhexyl 3-[3-amino-4-[(4-methylpiperazin-1-yl)methyl]phenyl]sulfanylpropanoate (Compound 28c) instead of 3-[(dimethylamino)methyl]-2-methyl-6-sulfanyl-phenol (Compound 16h), N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g). 7-[3-Amino-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (200.0 mg, Compound 28d) was obtained as a white solid. MS obsd. (ESI⁺) [(M+H)⁺]: 892.5.

### Step 5: Preparation of 7-[3-amino-4-[(4-methylpiperazin-1-yl)methyl]phenyl]sulfanyl-2-(ethoxymethyl)-6-methyl-1H-imidazo[4,5-c]pyridin-4-amine (Example 28)

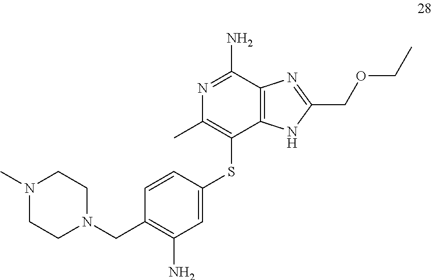 [SMILES: CCOCc1nc2c(N)nc(C)c(Sc3ccc(CN4CCN(C)CC4)c(N)c3)c2[nH]1]

A solution of 7-[3-amino-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (100.0 mg, Compound 28d, 0.11 mmol) and TFA (5.0 mL) in DCM (5.0 mL) was stirred at 20° C. for 2 h. The reaction mixture was concentrated in vacuo. The residue was purified by prep-HPLC and lyophilized to afford 7-[3-amino-4-[(4-methylpiperazin-1-yl)methyl]phenyl]sulfanyl-2-(ethoxymethyl)-6-methyl-1H-imidazo[4,5-c]pyridin-4-amine (29.34 mg, Example 28) as a yellow solid. MS obsd. (ESI⁺) [(M+H)⁺]: 442.3. ¹H NMR (400 MHz, CD₃OD) δ ppm: 6.88 (d, J=8.0 Hz, 1H), 6.37 (d, J=1.6 Hz, 1H), 6.36 (dd, J=8.0 Hz, 1.6 Hz, 1H), 4.65 (s, 2H), 3.64-3.56 (m, 2H), 3.48 (s, 2H), 3.26-3.04 (m, 4H), 2.78 (s, 3H), 2.71-2.59 (m, 4H), 2.58 (s, 3H), 1.20 (t, J=7.2 Hz, 3H).

### Example 29

### 7-[2-amino-6-methoxy-4-[(4-methylpiperazin-1-yl)methyl]phenyl]sulfanyl-2-(ethoxymethyl)-6-methyl-1H-imidazo[4,5-c]pyridin-4-amine

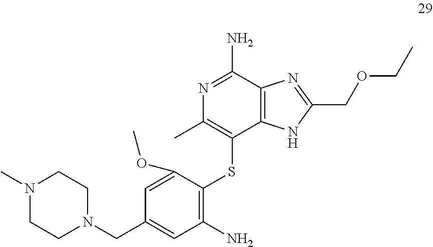 [SMILES: CCOCc1nc2c(N)nc(C)c(Sc3c(N)cc(CN4CCN(C)CC4)cc3OC)c2[nH]1]

### Step 1: Preparation of (4-formyl-2-methoxy-6-nitro-phenyl) trifluoromethanesulfonate (Compound 29a)

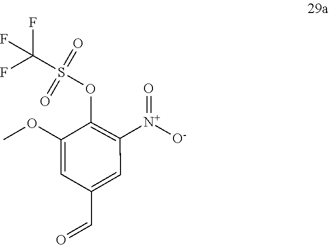 [SMILES: COc1cc(C=O)cc([N+](=O)[O-])c1OS(=O)(=O)C(F)(F)F]

To a solution of 5-nitrovanillin (5.0 g, 25.36 mmol) in DCM (60 mL) were added pyridine (3.01 g, 38.04 mmol) and trifluoromethanesulfonic anhydride (7.94 g, 28.15 mmol) were added at 0° C. The reaction mixture at 0° C. for 2 h. The mixture was diluted with DCM (150 mL), washed with water (80 mL) and brine (80 mL), dried over anhydrous sodium sulfate, filtered and concentrated in vacuo. The residue was purified by silica gel column and concentrated in vacuo to afford (4-formyl-2-methoxy-6-nitro-phenyl) trifluoromethanesulfonate (6.9 g, Compound 29a) as a white solid. ¹H NMR (400 MHz, CDCl₃) δ ppm: 10.04 (s, 1H), 8.14 (d, J=1.6 Hz, 1H), 7.83 (d, J=1.6 Hz, 1H), 4.10 (s, 3H).

### Step 2: Preparation of 2-ethylhexyl 3-(4-formyl-2-methoxy-6-nitro-phenyl)sulfanylpropanoate (Compound 29b)

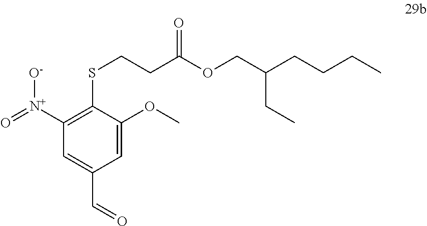 [SMILES: CCCCC(CC)COC(=O)CCSc1c(OC)cc(C=O)cc1[N+](=O)[O-]]

Compound 29b was prepared in analogy to Example 17, Step 1 by using (4-formyl-2-methoxy-6-nitro-phenyl) trifluoromethanesulfonate (Compound 29a) instead of 3-hydroxy-4-iodo-benzaldehyde. 2-Ethylhexyl 3-(4-formyl-2-methoxy-6-nitro-phenyl)sulfanylpropanoate (100.0 mg, Compound 29b) was obtained as a light yellow solid. ¹H NMR (400 MHz, CDCl₃) δ ppm: 9.98 (s, 1H), 7.77 (s, 1H), 7.55 (s, 1H), 4.07 (s, 3H), 4.02-3.93 (m, 2H), 3.25 (t, J=7.2 Hz, 2H), 2.54 (t, J=7.2 Hz, 2H), 1.56-1.52 (m, 1H), 1.34-1.23 (m, 8H), 0.93-0.85 (m, 6H).

### Step 3: Preparation of 2-ethylhexyl 3-[2-methoxy-4-[(4-methylpiperazin-1-yl)methyl]-6-nitro-phenyl]sulfanylpropanoate (Compound 29c)

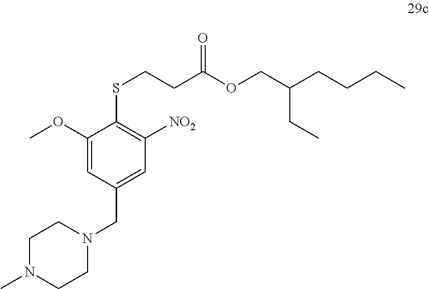 [SMILES: CCCCC(CC)COC(=O)CCSc1c(OC)cc(CN2CCN(C)CC2)cc1[N+](=O)[O-]]

Compound 29c was prepared in analogy to Example 17, Step 2 by using 2-ethylhexyl 3-(4-formyl-2-methoxy-6-nitro-phenyl)sulfanylpropanoate (Compound 29b) instead of 2-ethylhexyl 3-(4-formyl-2-hydroxy-phenyl)sulfanylpropanoate (Compound 17a) and 1-methylpiperazine instead of diethylamine. 2-Ethylhexyl 3-[2-methoxy-4-[(4-methylpiperazin-1-yl)methyl]-6-nitro-phenyl]sulfanylpropanoate (1.0 g, Compound 29c) was obtained as a light yellow oil. MS obsd. (ESI⁺) [(M+H)⁺]: 482.3.

### Step 4: Preparation of 2-ethylhexyl 3-[2-amino-6-methoxy-4-[(4-methylpiperazin-1-yl)methyl]phenyl]sulfanylpropanoate (Compound 29d)

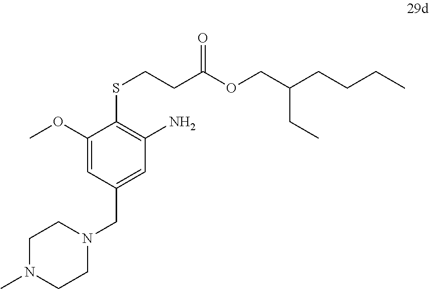 [SMILES: CCCCC(CC)COC(=O)CCSc1c(N)cc(CN2CCN(C)CC2)cc1OC]

Compound 29d was prepared in analogy to Example 20, Step 3 by using 2-ethylhexyl 3-[2-methoxy-4-[(4-methylpiperazin-1-yl)methyl]-6-nitro-phenyl]sulfanylpropanoate (Compound 29c) instead of 2-ethylhexyl 3-[4-[(dimethylamino)methyl]-2-nitro-phenyl]sulfanylpropanoate (Compound 20b). 2-Ethylhexyl 3-[2-amino-6-methoxy-4-[(4-methylpiperazin-1-yl)methyl]phenyl]sulfanylpropanoate (230.0 mg, Compound 29d) was obtained as a light red oil. MS obsd. (ESI⁺) [(M+H)⁺]: 452.3.

### Step 5: Preparation of 7-[2-amino-6-methoxy-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (Compound 29e)

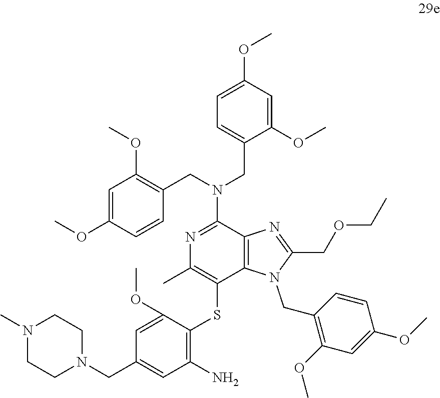

Compound 29e was prepared in analogy to Example 16, Step 9 by 2-ethylhexyl 3-[2-amino-6-methoxy-4-[(4-methylpiperazin-1-yl)methyl]phenyl]sulfanylpropanoate (Compound 29d) instead of 3-[(dimethylamino)methyl]-2-methyl-6-sulfanyl-phenol (Compound 16h) and N,N,1-tris[(2,4-dimethoxyphenyl)methyl]-2-(ethoxymethyl)-7-iodo-6-methyl-imidazo[4,5-c]pyridin-4-amine (Compound 8g) instead of 2-(ethoxymethyl)-7-iodo-6-methyl-1H-imidazo[4,5-c]pyridin-4-amine (Compound 1g). 7-[2-Amino-6-methoxy-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (230.0 mg, Compound 29e) was obtained as a light yellow oil. MS obsd. (ESI⁺) [(M+H)⁺]: 922.4.

### Step 6: Preparation of 7-[2-amino-6-methoxy-4-[(4-methylpiperazin-1-yl)methyl]phenyl]sulfanyl-2-(ethoxymethyl)-6-methyl-1H-imidazo[4,5-c]pyridin-4-amine (Example 29)

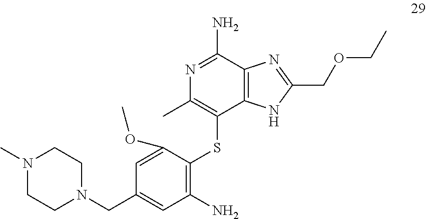 [SMILES: CCOCc1nc2c(N)nc(C)c(Sc3c(N)cc(CN4CCN(C)CC4)cc3OC)c2[nH]1]

A solution of 7-[2-amino-6-methoxy-4-[(4-methylpiperazin-1-yl)methyl]phenyl]sulfanyl-N,N,1-tris[(2,4-dimethoxyphenyl)methyl]-2-(ethoxymethyl)-6-methyl-imidazo[4,5-c]pyridin-4-amine (220.0 mg, Compound 29e, 0.24 mmol) in TFA (2.0 mL) was stirred at 25° C. for 2 h. The mixture was concentrated in vacuo. The residue was purified by prep-HPLC to afford 7-[2-amino-6-methoxy-4-[(4-methylpiperazin-1-yl)methyl]phenyl]sulfanyl-2-(ethoxymethyl)-6-methyl-1H-imidazo[4,5-c]pyridin-4-amine (34.67 mg, Example 29) as a white solid. MS obsd. (ESI⁺) [(M+H)⁺]: 472.3. ¹H NMR (400 MHz, CD₃OD) δ ppm: 6.60 (s, 1H), 6.41 (s, 1H), 4.73 (s, 2H), 3.71 (s, 3H), 3.68-3.61 (m, 2H), 3.60-3.32 (m, 4H), 3.23-2.93 (m, 4H), 2.85 (s, 3H), 2.79 (s, 3H), 2.65-2.22 (m, 2H), 1.29 (t, J=7.2 Hz, 3H).

### Example 30

### N-[5-[[4-amino-2-(ethoxymethyl)-6-methyl-1H-imidazo[4,5-c]pyridin-7-yl]sulfanyl]-2-pyridyl]-N′,N′-dimethyl-ethane-1,2-diamine

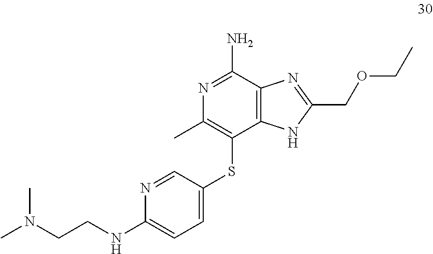 [SMILES: CCOCc1nc2c(N)nc(C)c(Sc3ccc(NCCN(C)C)nc3)c2[nH]1]

### Step 1: Preparation of N-(5-iodo-2-pyridyl)-N′,N-dimethyl-ethane-1,2-diamine (Compound 30a)

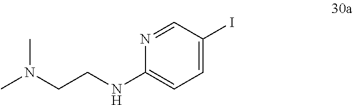

To a solution of 2-chloro-5-iodo-pyridine (1 g, 4.18 mmol) in DMSO (10.44 mL) was added 2-aminoethyl(dimethyl)amine (1.1 g, 1.37 mL, 12.53 mmol). The reaction was stirred at 100° C. for 1 h. The mixture was diluted with H₂O (20 mL and extracted with EA (3×20 mL). The combined organic layer was washed with brine (3×30 mL), dried over Na₂SO₄, filtered and concentrated in vacuo. The residue was purified by silica gel column and concentrated in vacuo to afford N-(5-iodo-2-pyridyl)-N,N-dimethyl-ethane-1,2-diamine (163 mg, Compound 30a) as a yellow oil. ¹H NMR (400 MHz, CDCl₃) δ ppm: 8.15 (d, J=2.0 Hz, 1H), 7.50 (dd, J=8.8 Hz, 2.0 Hz, 1H), 6.19 (d, J=8.8 Hz, 1H), 5.07 (br s, 1H), 3.28-3.22 (m, 2H), 2.50-2.43 (m, 2H), 2.19 (s, 6H).

### Step 2: Preparation of 6-[2-(dimethylamino)ethylamino]pyridine-3-thiol (Compound 30b)

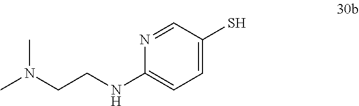

To a mixture of N-(5-iodo-2-pyridyl)-N,N-dimethyl-ethane-1,2-diamine (163 mg, Compound 30a, 0.52 mmol) and triisopropylsilanethiol (196.22 mg, 1.03 mmol) in 1,4-dioxane (5.0 mL), were added Pd XantPhos G3 (106.48 mg, 0.1 mmol) and Cs₂CO₃ (335.75 mg, 1.03 mmol) at 20° C. The mixture was degassed three times under N₂ and stirred at 100° C. for 2 h. The mixture was cooled to room temperature, and then filtered and the filtrate was concentrated in vacuo. The residue was purified by column to afford 6-[2-(dimethylamino)ethylamino]pyridine-3-thiol (91.5 mg, Compound 30b) as a light yellow solid. MS obsd. (ESI⁺) [(M+H)⁺]: 197.9.

### Step 3: Preparation of N-[5-[[4-amino-2-(ethoxymethyl)-6-methyl-1H-imidazo[4,5-c]pyridin-7-yl]sulfanyl]-2-pyridyl]-N′,N′-dimethyl-ethane-1,2-diamine (Example 30)

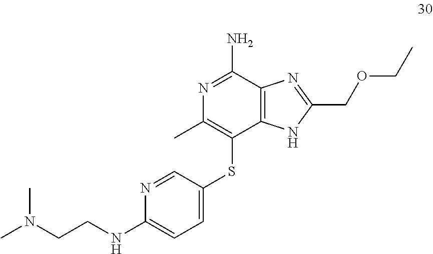 [SMILES: CCOCc1nc2c(N)nc(C)c(Sc3ccc(NCCN(C)C)nc3)c2[nH]1]

The title compound was prepared in analogy to Example 5, Step 3 by using 6-[2-(dimethylamino)ethylamino]pyridine-3-thiol (Compound 30b) instead of N-ethyl-N-[[4-[[4-[[ethyl(propyl)amino]methyl]phenyl]disulfanyl]phenyl]methyl]propan-1-amine (Compound 5b). N-[5-[[4-amino-2-(ethoxymethyl)-6-methyl-1H-imidazo[4,5-c]pyridin-7-yl]sulfanyl]-2-pyridyl]-N,N-dimethyl-ethane-1,2-diamine (1.5 mg, Example 30) was obtained as a white solid. MS obsd. (ESI⁺) [(M+H)⁺]: 402.1. ¹H NMR (400 MHz, CD₃OD) δ ppm: 8.08-8.03 (m, 1H), 7.42-7.37 (m, 1H), 6.49-6.43 (m, 1H), 4.62 (s, 2H), 3.59-3.52 (m, 4H), 2.82 (s, 6H), 2.64 (s, 3H), 2.13-2.06 (m, 1H), 1.96-1.90 (m, 1H), 1.14 (t, J=7.2 Hz, 3H).

### Example 31

### 7-[4-[2-(dimethylamino)ethyl]phenyl]sulfanyl-2-(ethoxymethyl)-6-methyl-1H-imidazo[4,5-c]pyridin-4-amine

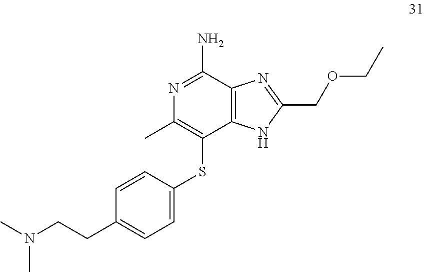 [SMILES: CCOCc1nc2c(N)nc(C)c(Sc3ccc(CCN(C)C)cc3)c2[nH]1]

### Step 1: Preparation of 2-ethylhexyl 3-[4-(2-oxoethyl)phenyl]sulfanylpropanoate (Compound 31a)

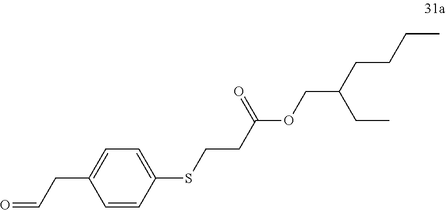 [SMILES: CCCCC(CC)COC(=O)CCSc1ccc(CC=O)cc1]

Compound 31a was prepared in analogy to Example 17, Step 1 by using 2-(4-bromophenyl)acetaldehyde instead of 3-hydroxy-4-iodo-benzaldehyde. 2-Ethylhexyl 3-[4-(2-oxoethyl)phenyl]sulfanylpropanoate (1.32 g, Compound 31a) was obtained as a yellow oil. ¹H NMR (400 MHz, CDCl₃) δ ppm: 9.65 (s, 1H), 7.44-7.30 (m, 2H), 7.19-7.07 (m, 2H), 4.06-3.96 (m, 2H), 3.70-3.63 (m, 2H), 3.25-3.11 (m, 2H), 2.72-2.58 (m, 2H), 1.63-1.54 (m, 1H), 1.41-1.25 (m, 8H), 0.93-0.84 (m, 6H).

### Step 2: Preparation of 2-ethylhexyl 3-[4-[2-(dimethylamino)ethyl]phenyl]sulfanylpropanoate (Compound 31b)

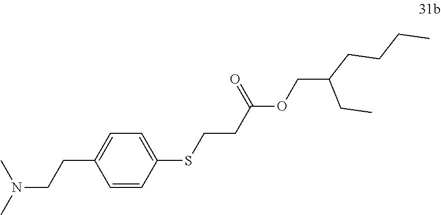 [SMILES: CCCCC(CC)COC(=O)CCSc1ccc(CCN(C)C)cc1]

Compound 31b was prepared in analogy to Example 17, Step 2 by using 2-ethylhexyl 3-[4-(2-oxoethyl)phenyl]sulfanylpropanoate (Compound 31a) instead of 2-ethylhexyl 3-(4-formyl-2-hydroxy-phenyl)sulfanylpropanoate (Compound 17a) and dimethylamine hydrochloride instead of diethylamine. 2-Ethylhexyl 3-[4-[2-(dimethylamino)ethyl]phenyl]sulfanylpropanoate (180.0 mg, Compound 31b) was obtained as a yellow oil. MS obsd. (ESI⁺) [(M+H)⁺]: 366.2.

### Step 3: Preparation of 7-[4-[2-(dimethylamino)ethyl]phenyl]sulfanyl-2-(ethoxymethyl)-6-methyl-1H-imidazo[4,5-c]pyridin-4-amine (Example 31)

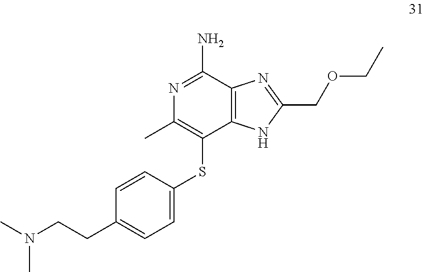 [SMILES: CCOCc1nc2c(N)nc(C)c(Sc3ccc(CCN(C)C)cc3)c2[nH]1]

The title compound was prepared in analogy to Example 16, Step 9 by using 2-ethylhexyl 3-[4-[2-(dimethylamino)ethyl]phenyl]sulfanylpropanoate (Compound 31b) instead of 3-[(dimethylamino)methyl]-2-methyl-6-sulfanyl-phenol (Compound 16h). 7-[4-[2-(Dimethylamino)ethyl]phenyl]sulfanyl-2-(ethoxymethyl)-6-methyl-1H-imidazo[4,5-c]pyridin-4-amine (10.33 mg, Example 31) was obtained as a green solid. MS obsd. (ESI⁺) [(M+H)⁺]: 386.1. ¹H NMR (400 MHz, CD₃OD) δ ppm: 7.18 (d, J=8.0 Hz, 2H), 7.02 (d, J=8.0 Hz, 2H), 4.64 (s, 2H), 3.65-3.56 (m, 2H), 3.29-3.25 (m, 2H), 2.98-2.94 (m, 2H), 2.87 (s, 6H), 2.55 (s, 3H), 1.21 (t, J=7.2 Hz, 3H).

### Example 32

### 6-[[4-amino-2-(ethoxymethyl)-6-methyl-1H-imidazo[4,5-c]pyridin-7-yl]sulfanyl]-2-methyl-3,4-dihydro-1H-isoquinolin-7-ol

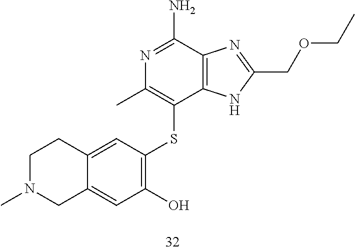 [SMILES: CCOCc1nc2c(N)nc(C)c(Sc3cc4c(cc3O)CN(C)CC4)c2[nH]1]

### Step 1: Preparation of 2-(3-bromo-4-methoxy-phenyl)ethanamine (Compound 32a)

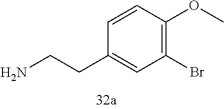

To a solution of 2-(3-bromo-4-methoxy-phenyl)acetonitrile (2.5 g, 11.06 mmol) in THF (25.0 mL) was added BH₃·THF (27.5 mL, 27.5 mmol, 1.0 M in THF) dropwise at 0° C. The mixture was stirred at 70° C. for 2 h. Then the mixture was cooled to 0° C. and HCl (3.0 mL) and MeOH (10 mL) were added. After being stirred at 70° C. for another 2 h, the mixture was cooled to room temperature and pH was adjusted to 9 with aq. NaOH (10 M). The mixture was diluted with H₂O (20 mL) and extracted with EA (3×20 mL). The combined organic layer was dried over Na₂SO₄, filtered and concentrated in vacuo. The residue was purified by silica gel column and concentrated in vacuo to afford 2-(3-bromo-4-methoxy-phenyl)ethanamine (1.2 g, Compound 32a) as a light yellow oil.

### Step 2: Preparation of methyl N-[2-(3-bromo-4-methoxy-phenyl)ethyl]carbamate (Compound 32b)

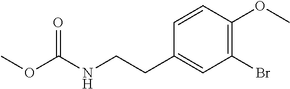

To a solution of triphosgene (709.3 mg, 2.39 mmol) in DCM (22.0 mL) was added 2-(3-bromo-4-methoxy-phenyl)ethanamine (1.1 g, Compound 32a, 4.78 mmol) slowly at 0° C. The mixture was stirred at 0° C. for 5 min, then NEt₃ (987.5 mg, 9.56 mmol) was added, the mixture was stirred at 0° C. for another 1 h. Then methanol (5.5 mL) and NEt₃ (987.5 mg, 9.56 mmol) were added to the mixture and the reaction was stirred at room temperature for 1 h. The mixture was washed with aq. NaCl (3×10 mL), dried over anhydrous Na₂SO₄, filtered and concentrated in vacuo to afford methyl N-[2-(3-bromo-4-methoxy-phenyl)ethyl]carbamate (1.75 g, Compound 32b) as a white solid, which was used directly to next step without further purification. MS obsd. (ESI⁺) [{⁷⁹Br}(M+H)⁺]: 287.9.

### Step 3: Preparation of 6-bromo-7-methoxy-3,4-dihydro-2H-isoquinolin-1-one (Compound 32c)

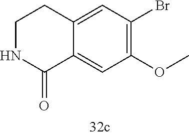

A mixture of methyl N-[2-(3-bromo-4-methoxy-phenyl)ethyl]carbamate (1.65 g, Compound 32b, 5.73 mmol) and phosphorus pentoxide in methanesulfonic acid (13.4 mL) was stirred at 90° C. for 2 h. Then the mixture was poured into ice water (110 mL) and extracted with EA (3×100 mL). The organic layer was dried over anhydrous Na₂SO₄, filtered and concentrated in vacuo. The residue was purified by column to afford 6-bromo-7-methoxy-3,4-dihydro-2H-isoquinolin-1-one (670 mg, Compound 32c) as a white solid. MS obsd. (ESI⁺) [{⁷⁹Br}(M+H)⁺]: 255.8.

### Step 4: Preparation of 6-bromo-7-methoxy-2-methyl-3,4-dihydroisoquinolin-1-one (Compound 32d)

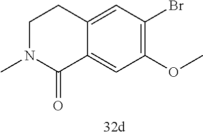

To a solution of 6-bromo-7-methoxy-3,4-dihydro-2H-isoquinolin-1-one (620 mg, Compound 32c, 2.42 mmol) in DCM (24.0 mL) was added NaH (116.4 mg, 2.91 mmol, 60%, dispersion in paraffin liquid) slowly at 0° C. The mixture was stirred at 0° C. for 45 min, then iodomethane (687.2 mg, 4.84 mmol) was added, and the mixture was stirred at room temperature for 1 h. Then the reaction was quenched by H₂O (20 mL) and extracted with EA (3×50 mL). The organic layer was dried over anhydrous Na₂SO₄, filtered and concentrated in vacuo. The residue was purified by column to afford 6-bromo-7-methoxy-2-methyl-3,4-dihydroisoquinolin-1-one (210 mg, Compound 32d) as a white solid. MS obsd. (ESI⁺) [{⁷⁹Br}(M+H)⁺]: 269.9.

### Step 5: Preparation of 6-bromo-7-methoxy-2-methyl-3,4-dihydro-1H-isoquinoline (Compound 32e)

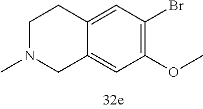

A mixture of 6-bromo-7-methoxy-2-methyl-3,4-dihydroisoquinolin-1-one (210 mg, Compound 32d, 0.78 mmol) and BH₃·THF (7.77 mL, 7.77 mmol, 1.0 M in THF) was stirred at 70° C. for 1 h. Then the mixture was quenched by MeOH and then concentrated in vacuo to afford 6-bromo-7-methoxy-2-methyl-3,4-dihydro-1H-isoquinoline (199 mg, Compound 32e) as a white solid, which was used directly to next step without further purification. MS obsd. [{⁷⁹Br}(M+H)⁺]: 255.9.

### Step 6: Preparation of 6-bromo-2-methyl-3,4-dihydro-1H-isoquinolin-7-ol (Compound 32f)

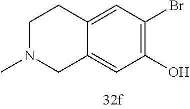

To a solution of 6-bromo-7-methoxy-2-methyl-3,4-dihydro-1H-isoquinoline (70 mg, Compound 32e, 0.27 mmol) in DCM (2.7 mL) was added BBr₃ (2.73 mL, 2.73 mmol, 1.0 M in THF) slowly at 0° C. The mixture was stirred at room temperature for 3 h. Then the mixture was quenched by MeOH and then concentrated in vacuo. DCM (5 mL) and NaOH (10.0 mL, 2 M in H₂O) were added into the mixture and the reaction was stirred at room temperature for 2 h. The pH of the mixture was adjusted to 7 by acetic acid and the mixture was extracted with DCM (3×20 mL). The organic layer was dried over anhydrous Na₂SO₄, filtered and concentrated in vacuo to afford 6-bromo-2-methyl-3,4-dihydro-1H-isoquinolin-7-ol (60 mg, Compound 32f) as a white solid, which was used directly to next step without further purification. MS obsd. [{⁷⁹Br}(M+H)⁺]: 241.9.

### Step 7: Preparation of 2-ethylhexyl 3-[(7-hydroxy-2-methyl-3,4-dihydro-1H-isoquinolin-6-yl)sulfanyl]propanoate (Compound 32g)

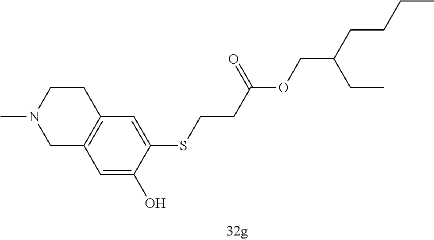 [SMILES: CCCCC(CC)COC(=O)CCSc1cc2c(cc1O)CN(C)CC2]

Compound 32g was prepared in analogy to Example 17, Step 1 by using 6-bromo-2-methyl-3,4-dihydro-1H-isoquinolin-7-ol (Compound 32f) instead of 3-hydroxy-4-iodo-benzaldehyde. 2-Ethylhexyl 3-[(7-hydroxy-2-methyl-3,4-dihydro-1H-isoquinolin-6-yl)sulfanyl]propanoate (100 mg, Compound 32g) was obtained as a light yellow solid. MS obsd. (EST⁺) [(M+H)⁺]: 380.2.

### Step 8: Preparation of 6-[[4-amino-2-(ethoxymethyl)-6-methyl-1H-imidazo[4,5-c]pyridin-7-yl]sulfanyl]-2-methyl-3,4-dihydro-1H-isoquinolin-7-ol (Example 32)

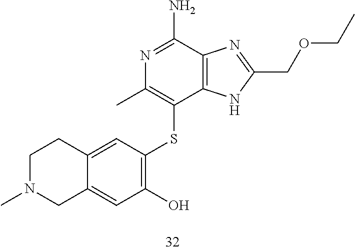 [SMILES: CCOCc1nc2c(N)nc(C)c(Sc3cc4c(cc3O)CN(C)CC4)c2[nH]1]

The title compound was prepared in analogy to Example 16, Step 9 by using 2-ethylhexyl 3-[(7-hydroxy-2-methyl-3,4-dihydro-1H-isoquinolin-6-yl)sulfanyl]propanoate (Compound 32g) instead of 3-[(dimethylamino)methyl]-2-methyl-6-sulfanyl-phenol (Compound 16h). 6-[[4-Amino-2-(ethoxymethyl)-6-methyl-1H-imidazo[4,5-c]pyridin-7-yl]sulfanyl]-2-methyl-3,4-dihydro-1H-isoquinolin-7-ol (36.9 mg, Example 32) was obtained as a light yellow solid. MS obsd. (ESI⁺) [(M+H)⁺]: 400.4. ¹H NMR (400 MHz, CD₃OD) δ=6.49 (s, 1H), 6.30 (s, 1H), 4.55 (s, 2H), 3.99 (s, 2H), 3.51 (q, J=7.2 Hz, 2H), 3.12 (t, J=6.4 Hz, 2H), 2.75-2.68 (m, 5H), 2.44 (s, 3H), 1.12 (t, J=7.2 Hz, 3H).

Reference Compound Compound SAN-43 as the Compound 43 disclosed in prior art WO2022167438A1 was selected as the reference compound for comparison.

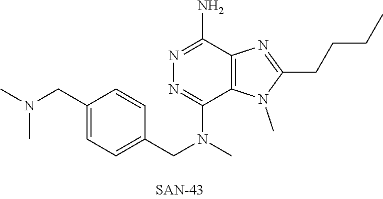 [SMILES: CCCCc1nc2c(N)nnc(N(C)Cc3ccc(CN(C)C)cc3)c2n1C]

### Example 33

### Activity of Compounds of this Invention in HEK293-Blue-hTLR7 Cells Assay and HEK293-Blue-hTLR8 Cells Assay

### HEK293-Blue-hTLR7 Cells Assay:

A stable HEK293-Blue-hTLR7 cell line was purchased from InvivoGen (Cat. #: hkb-htlr7, San Diego, California, USA). These cells were designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was regulated by the NF-κB promoter upon stimulation of human TLR7 for 20 hrs. The cell culture supernatant SEAP reporter activity was determined using QUANTI-Blue™ kit (Cat. #: rep-qbl, Invivogen, San Diego, Ca, USA) at a wavelength of 650 nm, a detection medium that turns purple or blue in the presence of alkaline phosphatase.

HEK293-Blue-hTLR7 cells were incubated at a density of 250,000˜450,000 cells/mL in a volume of 180 μL in a 96-well plate in Dulbecco's Modified Eagle's medium (DMEM) containing 4.5 g/L glucose, 100 U/mL penicillin, 100 μg/mL streptomycin, 100 μg/mL Normocin, 1% (V/V) GlutaMAX™, 10% (V/V) heat-inactivated fetal bovine serum for 24 hrs. Then the HEK293-Blue-hTLR-7 cells were incubated with addition of 20 μL test compound in a serial dilution in the presence of final DMSO at 1% and perform incubation under 37° C. in a CO₂ incubator for 20 hrs. Then 20 μL of the supernatant from each well was incubated with 180 μL Quanti-blue substrate solution at 37° C. for 1 hours and the absorbance was read at 620-655 nm using a spectrophotometer. The signalling pathway that TLR7 activation leads to downstream NF-κB activation has been widely accepted, and therefore similar reporter assay was also widely used for evaluating TLR7 agonist (Tsuneyasu Kaisho and Takashi Tanaka, Trends in Immunology, Volume 29, Issue 7, July 2008, Pages 329.sci; Hiroaki Hemmi et al, Nature Immunology 3, 196-200 (2002)).

### HEK293-Blue-hTLR8 Cells Assay:

A stable HEK293-Blue-hTLR8 cell line was purchased from InvivoGen (Cat. #: hkb-htlr8, San Diego, California, USA). These cells were designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was regulated by the NF-κB promoter upon stimulation of human TLR8. The cell culture supernatant SEAP reporter activity was determined using QUANTI-Blue™ kit (Cat. #: rep-qbl, Invivogen, San Diego, Ca, USA) at a wavelength of 650 nm, a detection medium that turns purple to blue in the presence of alkaline phosphatase.

HEK293-Blue-hTLR8 cells were incubated at a density of 250,000-450,000 cells/mL in a volume of 180 μL in a 96-well plate in Dulbecco's Modified Eagle's medium (DMEM) containing 4.5 g/L glucose, 100 U/mL penicillin, 100 μg/mL streptomycin, 100 μg/mL Normocin, 1% (V/V) GlutaMAX™, 10% (v/v) heat-inactivated fetal bovine serum for 24 hours. Then the HEK293-Blue-hTLR8 cells were incubated with addition of 20 μL test compound in a serial dilution in the presence of final DMSO at 1% and perform incubation under 37° C. in a CO₂ incubator for 20 hours. Then 20 μL of the supernatant from each well was incubated with 180 μL Quanti-blue substrate solution at 37° C. for 1 hours and the absorbance was read at 620-655 nm using a spectrophotometer. The signalling pathway that TLR8 activation leads to downstream NF-κB activation has been widely accepted, and therefore similar reporter assay was also widely used for evaluating TLR8 agonist.

The compounds of the present invention were tested in the above assay for their TLR7 and TLR8 agonism activities as described herein and results are listed in Table 1. Additional to the significant structure differences compared with reference compound SAN-43, the examples of this invention were found to be dual agonist having TLR7 EC₅₀ (0.001 μM˜0.048 μM) and TLR8 (0.003 μM˜0.082 μM), while compound SAN-43 did not show comparable TLR7 and TLR8 activities. Moreover, particular examples of this invention were found equally potent with both TLR7 and TLR8 activities (EC₅₀<0.03 μM).

TABLE 1
Activity of Compounds in HEK293-
hTLR7 and HEK293-hTLR8 assay:

| | HEK293-hTLR7 | HEK293-hTLR8 |
|---|---|---|
| Example No. | EC₅₀ (μM) | EC₅₀ (μM) |
| SAN-43 | 0.310 | 0.037 |
| 1 | 0.027 | 0.014 |
| 2 | 0.035 | 0.028 |
| 3 | 0.028 | 0.024 |
| 4 | 0.001 | 0.020 |
| 5 | 0.018 | 0.030 |
| 6 | 0.015 | 0.010 |
| 7 | 0.027 | 0.026 |
| 8 | 0.027 | 0.026 |
| 9 | 0.030 | 0.003 |
| 10 | 0.008 | 0.010 |
| 11 | 0.018 | 0.004 |
| 12 | 0.010 | 0.003 |
| 13 | 0.012 | 0.019 |
| 14 | 0.015 | 0.014 |
| 15 | 0.006 | 0.022 |
| 16 | 0.031 | 0.022 |
| 17 | 0.024 | 0.056 |
| 18 | 0.009 | 0.019 |
| 19 | 0.048 | 0.043 |
| 20 | 0.022 | 0.040 |
| 21 | 0.014 | 0.043 |
| 22 | 0.028 | 0.037 |
| 23 | 0.025 | 0.082 |
| 24 | 0.022 | 0.056 |
| 25 | 0.033 | 0.061 |
| 26 | 0.021 | 0.015 |
| 27 | 0.039 | 0.052 |
| 28 | 0.010 | 0.044 |
| 29 | 0.006 | 0.044 |
| 30 | 0.013 | 0.011 |
| 31 | 0.018 | 0.005 |
| 32 | 0.021 | 0.054 |

1. A compound of formula (I), 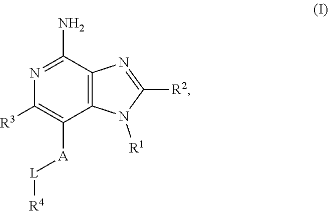
wherein
R¹ is H, C₁₋₆alkyl or hydroxyC₁₋₆alkyl;
R² is C₁₋₆alkyl or C₁₋₆alkoxyC₁₋₆alkyl;
R³ is C₁₋₆alkyl;
R⁴ is 1H-indazolyl substituted by (C₁₋₆alkyl)₂aminoC₁₋₆alkyl,
1H-indolyl substituted by (C₁₋₆alkyl)₂aminoC₁₋₆alkyl,
1H-isoquinolinyl twice substituted by C₁₋₆alkyl and hydroxy,
phenyl which is once, twice or three times substituted by substituents independently selected from (C₁₋₆alkyl)₂aminoC₁₋₆alkyl, (C₁₋₆alkylpiperazinyl)C₁₋₆alkyl, amino, C₁₋₆alkoxy, C₁₋₆alkyl, hydroxy and pyrrolidinylC₁₋₆alkyl, or
pyridyl substituted by (C₁₋₆alkyl)₂aminoC₁₋₆alkylamino;
A is S or Se;
L is a bond or C₁₋₆alkylene;
or a pharmaceutically acceptable salt thereof.

2. A compound according to claim 1, wherein R¹ is H or C₁₋₆alkyl.

3. A compound according to claim 1 or 2, wherein R¹ is H or methyl.

4. A compound according to any one of claims 1-3, wherein R² is butyl or ethoxymethyl.

5. A compound according to any one of claims 1-4, wherein R³ is methyl.

6. A compound according to any one of claims 1-5, wherein A is S.

7. A compound according to any one of claims 1-6, wherein L is a bond or ethylene.

8. A compound according to any one of claims 1-7, wherein R⁴ is phenyl which is once, twice or three times substituted by substituents independently selected from (C₁₋₆alkyl)₂aminoC₁₋₆alkyl, amino, C₁₋₆alkoxy, hydroxy and pyrrolidinylC₁₋₆alkyl.

9. A compound according to any one of claims 1-8, wherein R⁴ is phenyl which is once, twice or three times substituted by substituents independently selected from (dimethylamino)methyl, [ethyl(methyl)amino]methyl, [ethyl(propyl)amino]methyl, amino, hydroxy, methoxy and pyrrolidinylmethyl.

10. A compound according to any one of claims 1-8, wherein R⁴ is 2-[4-[(dimethylamino)methyl]-3-hydroxy-phenyl]ethyl, 2-[4-[(dimethylamino)methyl]phenyl]ethyl, 2-amino-4-(pyrrolidin-1-ylmethyl)phenyl, 4-[(dimethylamino)methyl]-2,6-dimethoxy-phenyl, 4-[(dimethylamino)methyl]-2-hydroxy-phenyl, 4-[(dimethylamino)methyl]-3-hydroxy-phenyl, 4-[(dimethylamino)methyl]phenyl, 4-[[ethyl(methyl)amino]methyl]phenyl or 4-[[ethyl(propyl)amino]methyl]phenyl.

11. A compound according to any one of claims 1-10, wherein
R¹ is H or C₁₋₆alkyl;
R² is C₁₋₆alkyl or C₁₋₆alkoxyC₁₋₆alkyl;
R³ is C₁₋₆alkyl;
R⁴ is phenyl which is once, twice or three times substituted by substituents independently selected from (C₁₋₆alkyl)₂aminoC₁₋₆alkyl, amino, C₁₋₆alkoxy, hydroxy and pyrrolidinylC₁₋₆alkyl;
A is S;
L is a bond or C₁₋₆alkylene;
or a pharmaceutically acceptable salt thereof.

12. A compound according to claim 11, wherein
R¹ is H or methyl;
R² is butyl or ethoxymethyl;
R³ is methyl;
R⁴ is 2-[4-[(dimethylamino)methyl]-3-hydroxy-phenyl]ethyl, 2-[4-[(dimethylamino)methyl]phenyl]ethyl, 2-amino-4-(pyrrolidin-1-ylmethyl)phenyl, 4-[(dimethylamino)methyl]-2,6-dimethoxy-phenyl, 4-[(dimethylamino)methyl]-2-hydroxy-phenyl, 4-[(dimethylamino)methyl]-3-hydroxy-phenyl, 4-[(dimethylamino)methyl]phenyl, 4-[[ethyl(methyl)amino]methyl]phenyl or 4-[[ethyl(propyl)amino]methyl]phenyl;
A is S;
L is a bond or ethylene;
or a pharmaceutically acceptable salt thereof.

13. A compound selected from:
7-[4-[(dimethylamino)methyl]phenyl]sulfanyl-2-(ethoxymethyl)-6-methyl-1H-imidazo[4,5-c]pyridin-4-amine;
1-[4-amino-2-butyl-7-[4-[(dimethylamino)methyl]phenyl]sulfanyl-6-methyl-imidazo[4,5-c]pyridin-1-yl]-2-methyl-propan-2-ol;
1-[4-amino-7-[4-[(dimethylamino)methyl]phenyl]selanyl-2-(ethoxymethyl)-6-methyl-imidazo[4,5-c]pyridin-1-yl]-2-methyl-propan-2-ol;
7-[4-[(dimethylamino)methyl]-2,6-dimethoxy-phenyl]sulfanyl-2-(ethoxymethyl)-6-methyl-1H-imidazo[4,5-c]pyridin-4-amine;
2-(ethoxymethyl)-7-[4-[[ethyl(propyl)amino]methyl]phenyl]sulfanyl-6-methyl-1H-imidazo[4,5-c]pyridin-4-amine;
2-(ethoxymethyl)-7-[4-[[ethyl(methyl)amino]methyl]phenyl]sulfanyl-6-methyl-1H-imidazo[4,5-c]pyridin-4-amine;
7-[4-[(dimethylamino)methyl]phenyl]sulfanyl-2-(ethoxymethyl)-1,6-dimethyl-imidazo[4,5-c]pyridin-4-amine;
2-[[4-amino-2-(ethoxymethyl)-6-methyl-1H-imidazo[4,5-c]pyridin-7-yl]sulfanyl]-5-[(dimethylamino)methyl]phenol;
7-[4-[(dimethylamino)methyl]-3-methoxy-phenyl]sulfanyl-2-(ethoxymethyl)-6-methyl-1H-imidazo[4,5-c]pyridin-4-amine;
7-[2-[4-[(dimethylamino)methyl]phenyl]ethylsulfanyl]-2-(ethoxymethyl)-6-methyl-1H-imidazo[4,5-c]pyridin-4-amine;
5-[[4-amino-2-(ethoxymethyl)-6-methyl-1H-imidazo[4,5-c]pyridin-7-yl]sulfanyl]-2-[(dimethylamino)methyl]phenol;
5-[4-amino-2-(ethoxymethyl)-1,6-dimethyl-imidazo[4,5-c]pyridin-7-yl]sulfanyl-2-[(dimethylamino)methyl]phenol;
5-[2-[[4-amino-2-(ethoxymethyl)-6-methyl-1H-imidazo[4,5-c]pyridin-7-yl]sulfanyl]ethyl]-2-[(dimethylamino)methyl]phenol;
2-butyl-7-[4-[(dimethylamino)methyl]phenyl]sulfanyl-6-methyl-1H-imidazo[4,5-c]pyridin-4-amine;
2-butyl-7-[2-[4-[(dimethylamino)methyl]phenyl]ethylsulfanyl]-6-methyl-1H-imidazo[4,5-c]pyridin-4-amine;
6-[[4-amino-2-(ethoxymethyl)-6-methyl-1H-imidazo[4,5-c]pyridin-7-yl]sulfanyl]-3-[(dimethylamino)methyl]-2-methyl-phenol;
2-[[4-amino-2-(ethoxymethyl)-6-methyl-1H-imidazo[4,5-c]pyridin-7-yl]sulfanyl]-5-(diethylaminomethyl)phenol;
7-[[4-[(dimethylamino)methyl]-1H-indol-7-yl]sulfanyl]-2-(ethoxymethyl)-6-methyl-1H-imidazo[4,5-c]pyridin-4-amine;
7-[[4-[(dimethylamino)methyl]-1H-indazol-7-yl]sulfanyl]-2-(ethoxymethyl)-6-methyl-1H-imidazo[4,5-c]pyridin-4-amine;
7-[2-amino-4-[(dimethylamino)methyl]phenyl]sulfanyl-2-(ethoxymethyl)-6-methyl-1H-imidazo[4,5-c]pyridin-4-amine;
2-[(4-amino-2-butyl-6-methyl-1H-imidazo[4,5-c]pyridin-7-yl)sulfanyl]-5-[(dimethylamino)methyl]phenol;
2-[[4-amino-2-(ethoxymethyl)-6-methyl-1H-imidazo[4,5-c]pyridin-7-yl]sulfanyl]-5-(pyrrolidin-1-ylmethyl)phenol;
2-[[4-amino-2-(ethoxymethyl)-6-methyl-1H-imidazo[4,5-c]pyridin-7-yl]sulfanyl]-5-[(dimethylamino)methyl]-4-methyl-phenol;
2-[[4-amino-2-(ethoxymethyl)-6-methyl-1H-imidazo[4,5-c]pyridin-7-yl]sulfanyl]-5-[1-(dimethylamino)ethyl]phenol;
5-[[4-amino-2-(ethoxymethyl)-6-methyl-1H-imidazo[4,5-c]pyridin-7-yl]sulfanyl]-2-[(dimethylamino)methyl]-4-methyl-phenol;
7-[2-amino-4-(pyrrolidin-1-ylmethyl)phenyl]sulfanyl-2-(ethoxymethyl)-6-methyl-1H-imidazo[4,5-c]pyridin-4-amine;
7-[2-amino-4-[(4-methylpiperazin-1-yl)methyl]phenyl]sulfanyl-2-(ethoxymethyl)-6-methyl-1H-imidazo[4,5-c]pyridin-4-amine;
7-[3-amino-4-[(4-methylpiperazin-1-yl)methyl]phenyl]sulfanyl-2-(ethoxymethyl)-6-methyl-1H-imidazo[4,5-c]pyridin-4-amine;
7-[2-amino-6-methoxy-4-[(4-methylpiperazin-1-yl)methyl]phenyl]sulfanyl-2-(ethoxymethyl)-6-methyl-1H-imidazo[4,5-c]pyridin-4-amine;
N-[5-[[4-amino-2-(ethoxymethyl)-6-methyl-1H-imidazo[4,5-c]pyridin-7-yl]sulfanyl]-2-pyridyl]-N,N-dimethyl-ethane-1,2-diamine;
7-[4-[2-(dimethylamino)ethyl]phenyl]sulfanyl-2-(ethoxymethyl)-6-methyl-1H-imidazo[4,5-c]pyridin-4-amine; and
6-[[4-amino-2-(ethoxymethyl)-6-methyl-1H-imidazo[4,5-c]pyridin-7-yl]sulfanyl]-2-methyl-3,4-dihydro-1H-isoquinolin-7-ol;
or a pharmaceutically acceptable salt thereof.

14. A process for the preparation of a compound according to any one of claims 1 to 13 comprising any one of the following steps:
a) the formation of compound of formula (I-1), 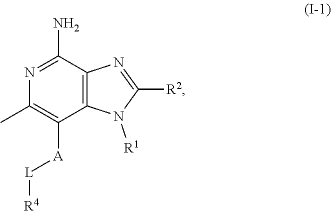
via reaction between compound of formula (III), 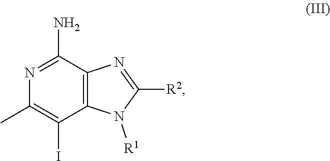
and compound of formula (II-1), WS-L-R⁴ (II-1), in the presence of a set of catalyst, ligand, reagent and base;
b) the formation of compound of formula (I-1) via deprotection of compound of formula (XIII), 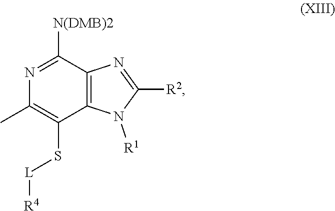
in the presence of an acid;
c) the formation of compound of formula (I-2), 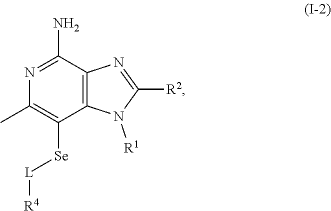
via the reaction between compound of formula (III) and compound of formula (II-2), B(OH)₂-L-R⁴ (II-2), in the presence of reagents;
wherein the set of catalyst, ligand, reagent and base in step a) is (1) Pd₂(dba)₃, XantPhos and DIEA (or Cs₂CO₃), (2) CuI, Mtpy and KOH, (3) Cu₂S, Fe and K₂CO₃ (or Cs₂CO₃), or (4) TIPSSH, XantPhos Pd G3 and K₂CO₃ (or Cs₂CO₃);
the acid in step b) is TFA or HCl;
the reagents in step c) are SeO₂ and KI;
wherein W is H, —S-L-R⁴ or R^{a}; R^{a} is alkoxycarbonylalkyl; R¹, R², and R⁴ are defined as in any one of claims 1 to 12.

15. A compound or a pharmaceutically acceptable salt thereof according to any one of claims 1 to 13, when manufactured according to the process of claim 14.

16. A pharmaceutical composition comprising a compound in accordance with any one of claims 1 to 13 and a pharmaceutically acceptable excipient.

17. A compound or pharmaceutically acceptable salt according to any one of claims 1 to 13 for use as therapeutically active substance.

18. The use of a compound according to any one of claims 1 to 13 as an agonist of TLR7 and TLR8.

19. The use of a compound according to any one of claims 1 to 13 for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

20. The use of a compound according to any one of claims 1 to 13 for the preparation of a medicament for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

21. The use according to claim 19 or 20, wherein the cancer is selected from pancreatic ductal adenocarcinoma and colorectal carcinoma.

22. A method for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma, which method comprises administering a therapeutically effective amount of a compound as defined in any one of claims 1 to 13.

23. The invention as hereinbefore described.
